(19) United States
(12) Patent Application Publication
(10) Pub. No.: US 2004/0005997 A1
(43) Pub. Date: Jan. 8, 2004

(54) Methods for identifying compounds for regulating muscle mass of function using amylin receptors

(75) Inventors: Robert Joseph Isfort, Fairfield, OH (US); Russell James Sheldon, Fairfield, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/443,201

(22) Filed: May 22, 2003

Related U.S. Application Data
(60) Provisional application No. 60/392,317, filed on Jun. 27, 2002.

(51) Int. Cl. G01N 33/53; G01N 33/567; A61K 38/17

(52) U.S. Cl. 514/2; 435/7.2

(57) ABSTRACT

Screening methods for identifying compounds that bind to or activate amylin receptors (AR) and regulate or potentially regulate skeletal muscle mass or function in vivo. Also disclosed are screening methods for identifying compounds that prolong or augment the activation of ARs or of AR signal transduction pathways, increase AR or increase amylin expression are provided. Pharmaceutical compositions comprising AR agonists, antibodies to AR and methods for increasing skeletal muscle mass or function or for the treatment of skeletal muscle atrophy using AR as the target for intervention and methods for treatment of muscular dystrophies are described.

### CROSS REFERENCE TO RELATED APPLICATION

This application claims the benefit of U.S. Provisional Application No. 60/392,317, filed Jun. 27, 2002, which is herein incorporated by reference in its entirety.

### TECHNICAL FIELD

The present invention relates to methods of identifying candidate compounds for regulating skeletal muscle mass or function or regulating the activity or expression of amylin receptors. The invention also relates to methods for the treatment of skeletal muscle atrophy or methods for inducing skeletal muscle hypertrophy using amylin receptors as the target for intervention and to methods of treating muscular dystrophies using amylin receptors as targets.

### BACKGROUND

Amylin Receptors and Ligands

Amylin, a 37 amino acid peptide, and as functionally and structurally related analogs has several physiological functions including cardiovascular, immunological, renal, neuronal and metabolic effects. Included in these physiological functions are vasodilation, glycogenolysis, modulation of glandular secretion, modulation of food intake, modulation of stomach emptying and modulation of cardiac output.

Amylin is the ligand/agonist of the amylin receptors. Amylin binds to and activates the amylin receptors.

There are two amylin receptors (AR) resulting from interaction of the same receptor membrane proteins, the calcitonin receptor and its splice variants, with two receptor activity modifying proteins, RAMP1 and RAMP3, identified to date which belong to G-protein coupled receptor (GPCR) class. Agonist activation of amylin receptors leads to G_{αs} activation of adenylate cyclase. Adenylate cyclase catalyzes the formation of cAMP, which in turn has multiple effects including the activation of protein kinase A, intracellular calcium release and activation of mitogen-activated protein kinase (MAP kinase).

Amylin receptors have been cloned from human, guinea pig, rabbit, pig, rat, and mouse. ARs each have unique distribution patterns. In humans three isoforms of the AR receptor have been cloned. Homologs for these three isoforms have been identified in other species.

The Amylin receptors can be pharmacologically distinguished from non-amylin receptors, through the use of receptor selective agonists and antagonists. These selective agonists and antagonist have been useful in evaluating the role of the amylin receptors in amylin mediated biological responses.

Skeletal Muscle Atrophy and Hypertrophy

Skeletal muscle is a plastic tissue that readily adapts to changes in physiological demand for work and metabolic need. Hypertrophy refers to an increase in skeletal muscle mass while skeletal muscle atrophy refers to a decrease in skeletal muscle mass. Acute skeletal muscle atrophy is traceable to a variety of causes including, but not limited to: disuse due to surgery, bed rest, or broken bones; denervation/nerve damage due to spinal cord injury, autoimmune disease, or infectious disease; glucocorticoid use for unrelated conditions; sepsis due to infection or other causes; nutrient limitation due to illness or starvation; and space travel. Skeletal muscle atrophy occurs through normal biological processes, however, in certain medical situations this normal biological process results in a debilitating level of muscle atrophy. For example, acute skeletal muscle atrophy presents a significant limitation in the rehabilitation of patients from immobilizations, including, but not limited to, those accompanying an orthopedic procedure. In such cases, the rehabilitation period required to reverse the skeletal muscle atrophy is often far longer than the period of time required to repair the original injury. Such acute disuse atrophy is a particular problem in the elderly, who may already suffer from substantial age-related deficits in muscle function and mass, because such atrophy can lead to permanent disability and premature mortality.

Skeletal muscle atrophy can also result from chronic conditions such as cancer cachexia, chronic inflammation, AIDS cachexia, chronic obstructive pulmonary disease (COPD), congestive heart failure, genetic disorders, e.g., muscular dystrophies, neurodegenerative diseases and sarcopenia (age associated muscle loss). In these chronic conditions, skeletal muscle atrophy can lead to premature loss of mobility, thereby adding to the disease-related morbidity.

Little is known regarding the molecular processes which control atrophy or hypertrophy of skeletal muscle. While the initiating trigger of the skeletal muscle atrophy is different for the various atrophy initiating events, several common biochemical changes occur in the affected skeletal muscle fiber, including a decrease in protein synthesis and an increase in protein degradation and changes in both contractile and metabolic enzyme protein isozymes characteristic of a slow (highly oxidative metabolism/slow contractile protein isoforms) to fast (highly glycolytic metabolism/fast contractile protein isoforms) fiber switch. Additional changes in skeletal muscle that occur include the loss of vasculature and remodeling of the extracellular matrix. Both fast and slow twitch muscle demonstrate atrophy under the appropriate conditions, with the relative muscle loss depending on the specific atrophy stimuli or condition. Importantly, all these changes are coordinately regulated and are switched on or off depending on changes in physiological and metabolic need.

The processes by which atrophy and hypertrophy occur are conserved across mammalian species. Multiple studies have demonstrated that the same basic molecular, cellular, and physiological processes occur during atrophy in both rodents and humans. Thus, rodent models of skeletal muscle atrophy have been successfully utilized to understand and predict human atrophy responses. For example, atrophy induced by a variety of means in both rodents and humans results in similar changes in muscle anatomy, cross-sectional area, function, fiber type switching, contractile protein expression, and histology. In addition, several agents have been demonstrated to regulate skeletal muscle atrophy in both rodents and in humans. These agents include anabolic steroids, growth hormone, insulin-like growth factor I, and beta-adrenergic agonists. Together, these data demonstrate that skeletal muscle atrophy results from common mechanisms in both rodents and humans.

Muscular dystrophies encompass a group of inherited, progressive muscle disorders, distinguished clinically by the selective distribution of skeletal muscle weakness. Treatment of muscular dystrophies with corticosteroids slows immune-mediated muscle fiber damage but also causes muscle atrophy.

While some agents have been shown to regulate skeletal muscle atrophy and are approved for use in humans for this indication, these agents have undesirable side effects such as hypertrophy of cardiac muscle, neoplasia, hirsutism, androgenization of females, increased morbidity and mortality, liver damage, hypoglycemia, musculoskeletal pain, increased tissue turgor, tachycardia, and edema. Currently, there are no highly effective and selective treatments for either acute or chronic skeletal muscle atrophy. Thus, there is a need to identify other therapeutic agents that regulate skeletal muscle atrophy.

### SUMMARY OF THE INVENTION

The present invention relates to the use of amylin receptors to identify candidate compounds that are potentially useful in the treatment of skeletal muscle atrophy and or to induce skeletal muscle hypertrophy. In particular, the invention provides in vitro methods for identifying candidate compounds for regulating skeletal muscle mass or function comprising contacting a test compound with a cell expressing amylin receptors, or contacting a test compound with isolated amylin receptors protein, and determining whether the test compound either binds to or activates amylin receptors. Another embodiment of the invention relates to a method for identifying candidate therapeutic compounds from a group of one or more candidate compounds which have been determined to bind to or activate amylin receptors comprising administering the candidate compound to a non-human animal and determining whether the candidate compound regulates skeletal muscle mass or muscle function in the treated animal.

A further embodiment of the invention relates to a method for identifying candidate compounds that selectively activate amylin receptor for regulating skeletal muscle mass or function comprising: (i) contacting a test compound with a cell expressing a functional amylin receptor, and determining a level of activation of amylin receptor resulting from the test compound; (ii) contacting a test compound with a cell expressing a functional calcitonin receptor, and determining the level of activation of calcitonin receptor resulting from the test compound and evaluation of the candidate compounds for regulating skeletal muscle mass or function.

The invention further provides methods for identifying candidate compounds that prolong or augment the agonist-induced activation of amylin receptors or of amylin receptor signal transduction pathway comprising: (i) contacting a test compound; with a cell which expresses a functional amylin receptor (ii) treating the cell with an amylin receptor agonist for a sufficient time and at a sufficient concentration to cause desensitization of the amylin receptor in control cells; followed by (iii) determining the level of activation of amylin receptors and identifying test compounds that prolong or augment the activation of an amylin receptor or an amylin receptor signal transduction pathway as candidate compounds for regulating skeletal muscle mass or function.

The invention further provides methods for identifying candidate compounds that increase amylin, or amylin receptor expression comprising contacting a test compound with a cell or cell lysate containing a reporter gene operatively associated with an amylin or amylin receptor gene regulatory element and detecting expression of the reporter gene. Test compounds that increase expression of the reporter gene are identified as candidate compounds for increasing expression.

The present invention also relates to the use of amylin receptor agonists, expression vectors encoding a functional amylin receptor, expression vectors encoding a constitutively active amylin receptor or compounds that increase expression of amylin receptors, or amylin to increase skeletal muscle mass or function or to treat skeletal muscle atrophy. In particular, the invention provides methods of treating skeletal muscle atrophy, in a subject in need of such treatment, comprising administering to the subject a safe and effective amount of an amylin receptor agonist, an expression vector encoding a functional amylin receptor, an expression vector encoding a constitutively active amylin receptor, an expression vector encoding amylin or an amylin analog, or a compound that increases expression of amylin receptor, or amylin. In a particular embodiment, the present invention relates to a method for treating skeletal muscle atrophy in a subject in need of such treatment comprising administering to the subject a safe and effective amount of an amylin receptor agonist in conjunction with a safe and effective amount of a compound that prolongs or augments the agonist-induced activation of amylin receptors, or of an amylin receptor signal transduction pathway.

The invention further provides for pharmaceutical compositions comprising a safe and effective amount of an amylin receptor agonist and a pharmaceutically acceptable carrier. In a particular embodiment the pharmaceutical composition comprises a chimeric or human antibody specific for an amylin receptor. In another particular embodiment the pharmaceutical composition comprises amylin or an amylin analog.

The present invention also provides for antibodies to amylin receptors and in particular to chimeric or human antibodies that are agonists of amylin receptors.

### SEQUENCE LISTING DESCRIPTION

Each of the amylin receptor nucleotide and protein sequences or amylin analog protein sequence included in the sequence listing, along with the corresponding Genbank or Derwent accession number(s) and animal species from which it is cloned, is shown in Table I. Also shown are accession numbers for related nucleotide sequences that encode identical, or nearly identical, amino acid sequences as the sequence shown in the sequence listing. These related sequences differ mainly in the amount of 5′ or 3′ untranslated sequence shown.

TABLE I

| | | | | |
|---|---|---|---|---|
| | SEQ ID | | Genbank (GB) or | Related Genbank |
| | NO: | | Derwent (D) | (GB) or Derwent |
| | nucleotide, | | Accession No. for | (D) Accession |
| Sequence Description | amino acid | Species | nucleotide sequence | Nos. |

Calcitonin Receptors:

| | | | | |
|---|---|---|---|---|
| calcitonin receptor | 1, 2 | human | U26553 (GB) | AB022177 (GB) |
| C1a | | | | AB022178 (GB) |
| calcitonin receptor | 3, 4 | human | X69920 (GB) | AAQ58997 (D) |
| C1a | | | | I72177 (GB) |
| | | | | I68246 (GB) |
| | | | | I67990 (GB) |
| | | | | I40808 (GB) |
| | | | | AAR51702 (D) |
| | | | | AAQ62283 (D) |
| | | | | I72200 (GB) |
| | | | | I68269 (GB) |
| | | | | I68013 (GB) |
| | | | | I40831 (GB) |
| | | | | AAR51704 (D) |
| | | | | AAQ42411 (D) |
| | | | | AAR37424 (D) |
| calcitonin receptor | 5, 6 | human | X82466 (GB) | AC003078 (GB) |
| C1a | | | | |
| calcitonin receptor | 7, 8 | human | U26554 (GB) | |
| C1a | | | | |
| calcitonin receptor | 9, 10 | human | L00587 (GB) | |
| C1b | | | | |
| calcitonin receptor | 11, 12 | human | I20773 (GB) | |
| C1b | | | | |
| calcitonin receptor | 13, 14 | human | AAQ62282 (D) | I72199 (GB) |
| C1b | | | | I68268 (GB) |
| | | | | I68012 (GB) |
| | | | | I40830 (GB) |
| | | | | AAB71868 (D) |
| calcitonin receptor | 15, 16 | human | U14637 (GB) | |
| C1b | | | | |
| calcitonin receptor | 17, 18 | guinea pig | U92463 (GB) | |
| calcitonin receptor | 19, 20 | rabbit | U66365 (GB) | |
| calcitonin receptor | 21, 22 | rabbit | U73126 (GB) | |
| isoform | | | | |
| calcitonin receptor | 23, 24 | pig | M74420 (GB) | Z31356 (GB) |
| calcitonin receptor | 25, 26 | rat | L13041 (GB) | |
| C1a | | | | |
| calcitonin receptor | 27, 28 | rat | L14617 (GB) | AAR62448 (D) |
| C1a | | | | |
| calcitonin receptor | 29, 30 | rat | L13040 (GB) | |
| C1b | | | | |
| calcitonin receptor | 31, 32 | rat | L14618 (GB) | AAR62447 (D) |
| C1b | | | | |
| calcitonin receptor | 33, 34 | rat | X70669 (GB) | |
| C1b partial CDS | | | | |
| calcitonin receptor | 35, 36 | mouse | AF056329 (GB) | |
| C1a | | | | |
| partial CDS | | | | |
| calcitonin receptor | 37, 38 | mouse | AF333473 (GB) | |
| C1a-1 | | | AF333474 (GB) | |
| | | | AF333475 (GB) | |
| | | | AF333476 (GB) | |
| | | | AF333477 (GB) | |
| | | | AF333478 (GB) | |
| | | | AF333479 (GB) | |
| | | | AF333481 (GB) | |
| | | | AF333482 (GB) | |
| | | | AF333483 (GB) | |
| | | | AF333484 (GB) | |
| | | | AF333485 (GB) | |
| calcitonin receptor | 39, 40 | mouse | AF333474 (GB) | |
| C1a-2 | | | AF333475 (GB) | |
| | | | AF333476 (GB) | |
| | | | AF333477 (GB) | |
| | | | AF333478 (GB) | |
| | | | AF333479 (GB) | |
| | | | AF333481 (GB) | |
| | | | AF333482 (GB) | |
| | | | AF333483 (GB) | |
| | | | AF333484 (GB) | |
| | | | AF333485 (GB) | |
| calcitonin receptor | 41, 42 | mouse | U18542 (GB) | AF333473 (GB) |
| C1b-1 | | | | AF333474 (GB) |
| | | | | AF333475 (GB) |
| | | | | AF333476 (GB) |
| | | | | AF333477 (GB) |
| | | | | AF333478 (GB) |
| | | | | AF333479 (GB) |
| | | | | AF333480 (GB) |
| | | | | AF333481 (GB) |
| | | | | AF333482 (GB) |
| | | | | AF333483 (GB) |
| | | | | AF333484 (GB) |
| | | | | AF333485 (GB) |
| calcitonin receptor | 43, 44 | mouse | AF333474 (GB) | |
| C1b-2 | | | AF333475 (GB) | |
| | | | AF333476 (GB) | |
| | | | AF333477 (GB) | |
| | | | AF333478 (GB) | |
| | | | AF333479 (GB) | |
| | | | AF333480 (GB) | |
| | | | AF333481 (GB) | |
| | | | AF333482 (GB) | |
| | | | AF333483 (GB) | |
| | | | AF333484 (GB) | |
| | | | AF333485 (GB) | |

RAMP 1 Sequences:

| | | | | |
|---|---|---|---|---|
| RAMP 1 | 45, 46 | human | AJ001014 (GB) | ABA09253 (D) |
| | | | | AC016776 (GB) |
| | | | | AC012076 (GB) |
| RAMP 1 | 47, 48 | human | BC000548 (GB) | |
| RAMP 1 | 49, 50 | rat | AF181550 (GB) | AB042887 (GB) |
| RAMP 1 | 51, 52 | rat | AB030942 (GB) | |
| RAMP 1 | 53, 54 | mouse | AJ314840 (GB) | |
| RAMP 1 | 55, 56 | mouse | AJ250489 (GB) | AF209904 (GB) |
| | | | | AF146522 (GB) |
| RAMP 1 | 57, 58 | mouse | BC012644 (GB) | AB019046 (GB) |
| RAMP 1 partial CDS | 59, 60 | pig | AF312385 (GB) | |

RAMP 3 Sequences:

| | | | | |
|---|---|---|---|---|
| RAMP 3 | 61, 62 | human | AJ001016 (GB) | AAZ98148 (D) |
| | | | | AAA80608 (D) |
| | | | | AAD08854 (D) |
| | | | | AAY87263 (D) |
| | | | | AAB25578 (D) |
| RAMP 3 | 63, 64 | mouse | AF209907 (GB) | AF146524 (GB) |
| | | | | AJ250491 (GB) |
| RAMP 3 | 65, 66 | rat | AB030944 (GB) | AB028935 (GB) |
| RAMP 3 | 67, 68 | rat | AB042889 (GB) | |
| RAMP 3 | 69, 70 | rat | AF181552 (GB) | |

### BRIEF DESCRIPTION OF THE FIGURES

FIG. 1 demonstrates the anti-atrophy effect of the AR agonist, amylin (administered subcutaneously, 2× daily), on the medial gastrocnemius muscle in the mouse sciatic nerve denervation atrophy model.

FIGS. 2A and 2B demonstrate the anti-atrophy effect of amylin (administered subcutaneously, 2× daily) on glucocorticoid-induced atrophy of the tibialis anterior muscle (FIG. 2A) and the medial gastrocnemius muscle (FIG. 2B).

FIGS. 3A and 3B demonstrate the anti-atrophy effect of amylin (administered subcutaneously, 2× daily) on the casting-induced atrophy of the tibialis anterior muscle and hypertrophy-inducing effect on the non-casted (normal) tibialis anterior muscle (FIG. 3A) and on the casting-induced atrophy of the medial gastrocnemius muscle and the hypertrophy inducing effect of amylin on the non-casted (normal) medial gastrocnemius muscle (FIG. 3B).

### DETAILED DESCRIPTION OF THE INVENTION

Terms and Definitions:

The following is a list of definitions for terms used herein.

“Agonist” means any compound that activates a receptor. For example, amylin receptor agonists include, but are not limited to, amylin and amylin analogs.

“Allelic variant” means a variant form of a given gene or gene product. One of skill in the art recognizes that a large number of genes are present in two or more allelic forms in a population and some genes have numerous alleles.

“Antibody” means immunoglobulin molecules or immunologically active portions them, i.e., molecules that contain an antigen binding site which specifically binds an antigen.

“Binding affinity” means the propensity for a ligand to interact with a receptor and is inversely related to the dissociation constant for a specific amylin ligand-amylin receptor interaction. The dissociation constant can be measured directly via standard saturation, competition, or kinetics binding techniques or indirectly via pharmacological techniques involving functional assays and endpoints.

“Chimeric antibody” means an antibody that contains structural elements from two or more types antibody molecules. Chimeric antibodies include, but are not limited to, antibodies known as “humanized antibodies”.

“Amylin analogs” means substances which act as ligands of amylin receptors. Suitable amylin analogs can be obtained from a variety of vertebrate species and include, but are not limited to, substances such as calcitonin gene related peptide, human calcitonin), adrenomedullin, salmon calcitonin and the amylin analogs. Preferred amylin analogs are calcitonin gene related peptide, salmon calcitonin, human calcitonin, and adrenomedullin.

“Amylin receptor agonist” means a compound or molecule that has the ability to activate any amylin receptor including amylin receptor 1 (calcitonin receptor+RAMP1) or amylin receptor 2 (calcitonin receptor+RAMP3), or both. Activation of amylin receptors can be measured as described hereinafter.

“Amylin receptor” (AR) means amylin receptor 1 (AR 1) or amylin receptor 2 (AR2).

“AR1” means any isoforms of amylin receptor 1 (calcitonin receptor+RAMP1) from any animal species.

The definition of AR1 includes, but is not limited to, those receptors for which the cDNA or genomic sequence encoding the receptor has been deposited in a sequence database. The nucleotide and protein sequences of these receptors are available from GenBank or Derwent and for convenience representative sequences are given in the sequence listing herein. (Accession No. AJ001014).

“AR2” means any isoform of amylin receptor 2 (calcitonin receptor+RAMP3) from any animal species.

The definition of AR2 receptor includes, but is not limited to, those receptors for which the DNA sequence encoding the receptor has been deposited in a sequence database. The nucleotide and protein sequences of these receptors are available from GenBank or Derwent and for convenience, representative sequences are given in the sequence listing herein. (Accession No. AJ001016).

The term “Amylin Receptor or AR” also includes truncated and/or mutated proteins wherein regions of the receptor molecule not required for ligand binding or signaling have been deleted or modified. For example one of skill in the art will recognize that a AR with one or more conservative changes in the primary amino acid sequence would be useful in the present invention. It is known in the art that substitution of certain amino acids with different amino acids with similar structure or properties (conservative substitutions) can result in a silent change, i.e., a change that does not significantly alter function. Conservative substitutes are well known in the art. For example, it is known that GPCRs can tolerate substitutions of amino acid residues in the transmembrane alpha-helices, which are oriented toward lipid, with other hydrophobic amino acids, and remain functional. AR1s differing from a naturally occurring sequence by truncations and/or mutations such as conservative amino acid substitutions are also included in the definition of AR1. AR2s differing from a naturally occurring sequence by truncations and/or mutations such as conservative amino acid substitutions are also included in the definition of AR2.

One of skill in the art would also recognize that ARs from a species other than those listed above, particularly mammalian species, would be useful in the present invention. One of skill in the art would further recognize that by using probes from the known AR species' sequences, cDNA or genomic sequences homologous to the known sequence could be obtained from the same or alternate species by known cloning methods. Such AR1s are also included in the definition of AR1 and such AR2s are also included in the definition of AR.

In addition, one of skill in the art would recognize that functional allelic variants or functional splice variants of ARs might be present in a particular species and that these variants would have utility in the present invention. Splice variants of ARs are known, for example U.S. Pat. Nos. 5,683,884, 5,674,981, 5,674,689, 5,622,839, and 5,516,651, each of which is incorporated herein by reference. Such AR1 variants are also included in the definition of AR1 and such AR2 variants are also included in the definition of AR2.

Fusions of an AR polypeptide, or an AR polypeptide fragment to a non-AR polypeptide are referred to as AR fusion proteins. Using known methods, one of skill in the art would be able to make fusion proteins of an AR that, while different from native AR, would remain useful in the present invention. For example the non-AR polypeptide may be a signal (or leader) polypeptide sequence that co-translationally or post-translationally directs transfer of the protein from its site of synthesis to another site (e.g., the yeast α-factor leader). Or the non-AR polypeptide may be added to facilitate purification or identification of the AR (e.g., poly-His, or Flag peptide). AR1 fusion proteins are also included within the definition of AR fusion proteins; AR2 fusion proteins are also included within the definition of AR.

“Inhibit” means to partially or completely block a particular process or activity. For example, a compound inhibits skeletal muscle atrophy if it either completely or partially prevents muscle atrophy.

As used herein, two DNA sequences are said to be “operably associated” if the nature of the linkage between the two DNA sequences does not (1) result in the introduction of a frame-shift mutation, (2) interfere with the ability of a promoter region to direct the transcription of the coding sequences, or (3) interfere with the ability of the corresponding RNA transcript to be translated into a protein. For example, a coding sequence and regulatory sequences are operably associated when they are covalently linked in such a way as to place the transcription of the coding sequence under the influence or control of the regulatory sequences. Thus, a promoter region is operably associated with a coding sequence when the promoter region is capable of effecting transcription of that DNA sequence such that the resulting transcript is capable of being translated into the desired protein or polypeptide.

“Percent identity” means the percentage of nucleotides or amino acids that two sequences have in common, calculated as follows. To calculate the percent identity for a specific sequence (the query), the relevant part of the query sequence is compared to a reference sequence using the BestFit comparison computer program, Wisconsin Package, Version 10.1, available from the Genetics Computer Group, Inc. This program uses the algorithm of Smith and Waterman, Advances in Applied Mathematics, Issue 2: 482-489 (1981). Percent identity is calculated with the following default parameters for the BestFit program: the scoring matrix is blosum62.cmp, the gap creation penalty is 8 and the gap extension penalty is 2. When comparing a sequence to the reference sequence, the relevant part of the query sequence is that which is derived from an AR sequence. For example, where the query is an AR/purification tag fusion protein, only the AR polypeptide portion of the sequence is aligned to calculate the percent identity score.

“Prophylactic treatment” means preventive treatment of a subject, not currently exhibiting signs of skeletal muscle atrophy or wasting, in order to completely or partially block the occurrence of skeletal muscle atrophy. One of skill in the art would recognize that certain individuals are at risk for skeletal muscle atrophy as discussed in the background section herein. Furthermore, one of skill in the art would recognize that if the biochemical changes leading to skeletal muscle atrophy are appropriately regulated, that the occurrence of atrophy would be prevented or reduced in at-risk individuals. For example, muscular dystrophy patients beginning treatment with corticosteroids are at risk for developing skeletal muscle atrophy indicating that prophylactic treatment of such patients would be appropriate.

“Regulatory element” means a DNA sequence that is capable of controlling the level of transcription from an operably associated DNA sequence. Included within this definition of regulatory element are promoters and enhancers. E.g., an AR gene regulatory element is a DNA sequence capable of controlling the level of transcription from the AR gene.

“Reporter gene” means a coding sequence whose product can be detected, preferably quantitatively, wherein the reporter gene is operably associated with a heterologous promoter or enhancer element that is responsive to a signal that is to be measured. The promoter or enhancer element in this context is referred to herein as a “responsive element”.

“Selective agonist” means that the agonist has significantly greater activity toward a certain receptor(s) compared with other receptors, not that it is completely inactive with regard to other receptors. A selective agonist for a specific receptor may show 10-fold, preferably 100-fold, more preferably 1000-fold and most preferably greater than 1000-fold selectivity toward that receptor than other related or unrelated receptors.

“Splice variant” means an mRNA or protein which results from alternative exon usage. One of skill in the art recognizes that, depending on cell type, or even within a single cell type, a mRNA may be expressed in a different form, as a splice variant, and thus the translated protein will be different depending upon the mRNA that is expressed.

A “therapeutically effective amount” of a substance is an amount capable of producing a medically desirable result in a treated patient, e.g., decreases skeletal muscle atrophy, increases skeletal muscle mass or increases skeletal muscle function, with an acceptable benefit: risk ratio; in a human or non-human mammal.

“Therapeutic treatment” means treatment of a subject in which an increase in muscle mass or muscle function is desirable. For example, treatment of a subject currently exhibiting signs of skeletal muscle atrophy in order to partially or completely reverse the skeletal muscle atrophy that has occurred or to completely or partially block the occurrence of further skeletal muscle atrophy would be therapeutic treatment of that subject. The term “therapeutic treatment” also includes, for example, treatment of a subject not exhibiting signs of skeletal muscle atrophy to induce skeletal muscle hypertrophy, e.g., treatment of a livestock animal to increase muscle mass.

The term “treatment” means prophylactic or therapeutic treatment.

The Role of ARs in Regulation of Skeletal Muscle Mass

One of skill in the art would recognize the utility of the present invention given the information in the prior art and the teachings below. The results described herein demonstrate that administration of an AR agonist, amylin, blocks and/or inhibits the skeletal muscle atrophy inducing effect of denervation, disuse or dexamethasone treatment in models of skeletal muscle atrophy. Together, these data demonstrate the modulatory role of ARs in the process of skeletal muscle atrophy. The specific role of ARs in vivo was investigated using the pharmacological agent amylin which is a selective agonist for ARs in various models of skeletal muscle atrophy, described hereinafter. Amylin has been well characterized and is described in the scientific literature.

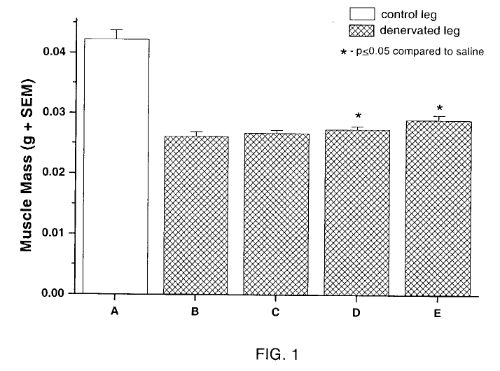

FIGS. 1-3 show the results of experiments demonstrating that administration of amylin, a selective agonist of ARs, results in statistically significant inhibition of skeletal muscle atrophy and induces skeletal muscle hypertrophy. Amylin administered twice daily in combination with the phosphodiesterase inhibitor, theophylline, resulted in inhibition of skeletal muscle atrophy in animal models of skeletal muscle atrophy. Theophylline administered alone in these atrophy models had no effect, demonstrating that the anti-atrophy effect of amylin in combination with theophylline was due to the effect of amylin. Statistical significance of the results was determined using ANCOVA (Douglas C. Montgomery, Design and Analysis of Experiments, John Wiley and Sons, New York (2^{nd} ed. 1984)). Abbreviations used in FIGS. 1-3: g-gram; SEM-standard error of the mean.

Specifically, FIG. 1 (FIG. 1.) shows that amylin inhibits denervation-induced atrophy of the medial gastrocnemius muscle in a mouse sciatic nerve denervation atrophy model. Legend: A—non-denervated medial gastrocnemius muscle from mice treated with physiological saline (non-atrophied control); B—denervated medial gastrocnemius muscle from mice treated with physiological saline (atrophied control); C—amylin (0.03 mg/kg)+theophylline; D—amylin (0.1 mg/kg)+theophylline; E—amylin (0.3 mg/kg)+theophylline. * —p≦0.05 compared to saline. Following denervation of the right sciatic nerve, male mice were injected subcutaneously in the midscapular region twice daily with amylin, at the doses indicated above or vehicle control (physiological saline) for nine days. Amylin was co-administered with twice daily intra-peritoneal dosing of the phosphodiesterase inhibitor theophylline (30 mg/kg). On day nine, the medial gastrocnemius muscle was removed and weighed to determine the degree of atrophy.

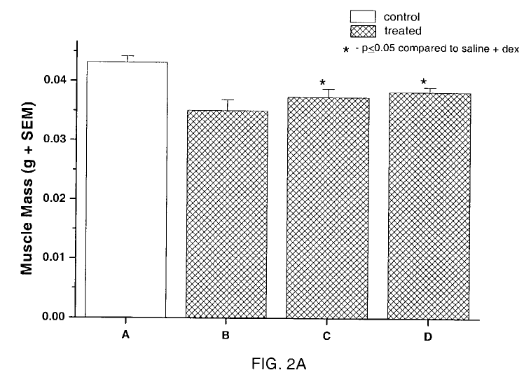

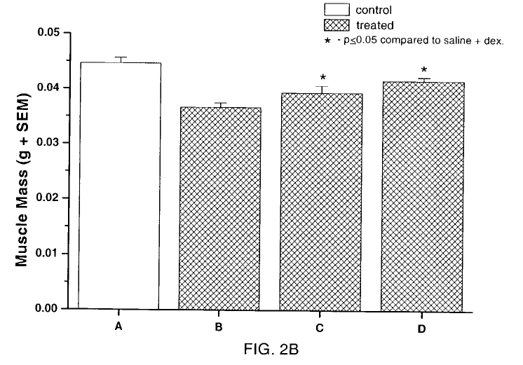

FIG. 2 (FIG. 2.) demonstrates that amylin inhibits glucocorticoid-induced muscle atrophy of the tibialis anterior (FIG. 2A) and medial gastrocnemius muscles (FIG. 2B) in the mouse glucocorticoid-induced atrophy model. Legend: A—water only with no dexamethasone included in drinking water (non-atrophied control); B—water+dexamethasone (atrophied control); C—amylin (0.3 mg/kg/d)+theophylline+dexamethasone; D—amylin (1.0 mg/kg/d)+theophylline+dexamethasone; *—p≦0.05 compared to water; #—p≦0.05 compared to water+dexamethasone. Following the addition of the glucocorticoid, dexamethasone, to the drinking water (1.2 mg/kg/d), male mice were injected subcutaneously in the midscapular region twice daily with amylin, at the doses indicated above or vehicle control (physiological saline) for nine days. Amylin was co-administered with twice daily intra-peritoneal dosing of the phosphodiesterase inhibitor theophylline (30 mg/kg). Nine days following the initiation of dosing amylin, the medial gastrocnemius and tibialis anterior muscles were removed and weighed to determine the degree of atrophy.

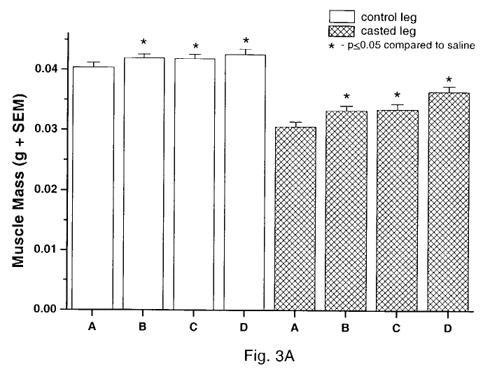

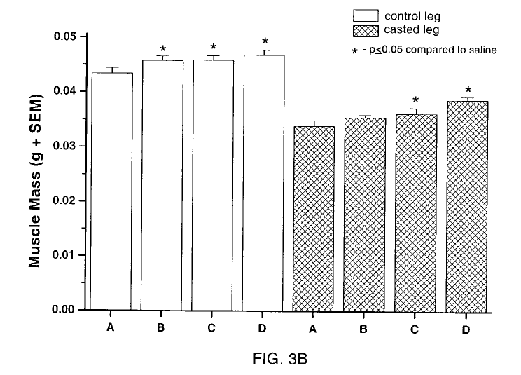

FIG. 3 (FIG. 3.) demonstrates that amylin inhibits disuse-induced atrophy of the tibialis anterior (FIG. 3A) and medial gastrocnemius (FIG. 3B) muscles. In addition, statistically significant hypertrophy of the medial gastrocnemius and tibialis anterior muscles of the non-casted leg was also observed with amylin treatment. Legend: A—physiological saline (control); B—amylin (0.1 mg/kg)+theophylline; C—amylin (0.3 mg/kg)+theophylline; D—amylin (1.0 mg/kg)+theophylline; *—p≦0.05 compared to saline. Following casting of the right hind leg, male mice were injected subcutaneously in the midscapular region twice daily, with amylin or vehicle control (physiological saline) for ten days at the daily delivered dose indicated. Amylin was co-administered with twice daily intra-peritoneal dosing of the phosphodiesterase inhibitor theophylline (30 mg/kg). On day ten, the medial gastrocnemius and tibialis anterior muscles were removed and weighed to determine the degree of atrophy.

III. Preparation of ARs, Amylin or Amylin Analogs, or Cell Lines Expressing ARs

ARs, amylin and amylin analogs can be prepared for a variety of uses, including, but not limited to, the generation of antibodies, use as reagents in the screening assays of the present invention, and use as pharmaceutical reagents for the treatment of skeletal muscle atrophy. It will be clear to one of skill in the art that, for certain embodiments of the invention, purified polypeptides will be most useful, while for other embodiments cell lines expressing the polypeptides will be most useful. For example, in situations where it is important to retain the structural and functional characteristics of the AR, e.g., in a screening method to identify candidate compounds that activate ARs, it is desirable to use cells that express functional ARs.

Because amylin and amylin analogs are short polypeptides, the skilled artisan will recognize that these polypeptides will be most conveniently provided by direct synthesis, rather than by recombinant means, using techniques well known in the art. In addition, many of these molecules are commercially available.

Where the source of ARs is a cell line expressing the polypeptide, the cells may, for example, endogenously express AR, have been stimulated to increase endogenous AR expression or have been genetically engineered to express an AR. Methods for determining whether a cell line expresses a polypeptide of interest are known in the art, for example, detection of the polypeptide with an appropriate antibody, use of a DNA probe to detect mRNA encoding the protein (e.g., northern blot or PCR techniques), or measuring binding of an agent selective for the polypeptide of interest (e.g., a radiolabeled selective agonist).

The use of recombinant DNA technology in the preparation of ARs, or of cell lines expressing these polypeptides is particularly contemplated. Such recombinant methods are well known in the art. To express recombinant ARs, an expression vector that comprises a nucleic acid that encodes the polypeptides of interest under the control of one or more regulatory elements, is prepared. Genomic or cDNA sequences encoding ARs from several species have been described and are readily available from the GenBank database or Derwent database as well as in the sequence listing for this application. The accession numbers for AR sequences and corresponding SEQ ID NOS. are shown in Table I. Using this publicly available sequence information, one means of isolating a nucleic acid molecule encoding a CR, Ramp 1 or Ramp 3 component of an AR is to screen a genomic DNA or cDNA library with a natural or artificially synthesized DNA probe, using methods well known in the art, e.g., by PCR amplification of the sequence from an appropriate library. Another method is to use oligonucleotide primers specific for the receptor of interest to PCR amplify the cDNA directly from mRNA isolated from a particular tissue (such as skeletal muscle). Such isolated mRNA is commercially available. One of skill in the art would also recognize that by using nucleic acid probes corresponding to portions of the known AR receptor sequences the homologous cDNAs or genomic sequences from other species can be obtained using known methods. Particularly useful in the methods of the present invention are AR receptors from the species including, but not limited to, guinea pig, rabbit, pig, rat, mouse and turkey. By methods well known in the art, the isolated nucleic acid molecule encoding the AR of interest is then ligated into a suitable expression vector. The expression vector, thus prepared, is expressed in a host cell and the host cells expressing the receptor are used directly in a screening assay or the receptor is isolated from the host cells expressing the receptor and the isolated receptor is used in a screening assay.

The host-expression vector systems that may be used for purposes of the invention include, but are not limited to: microorganisms such as bacteria (e.g., E. coli, B. subtilis) transformed with recombinant bacteriophage DNA, plasmid DNA, or cosmid DNA expression vectors containing AR nucleotide sequences; yeast (e.g., Saccharomyces, Pichia) transformed with recombinant yeast expression vectors containing AR nucleotide sequences; insect cell systems infected with recombinant virus expression vectors (e.g., baculovirus) containing AR nucleotide sequences; plant cell systems infected with recombinant virus expression vectors (e.g., cauliflower mosaic virus, tobacco mosaic virus) or transformed with recombinant plasmid expression vectors (e.g., Ti plasmid) containing AR nucleotide sequences; or mammalian cell systems (e.g., COS, CHO, HEK293, NIH3T3) harboring recombinant expression constructs containing promoters derived from the genome of mammalian cells (e.g., metallothionein promoter) or from mammalian viruses (e.g., retrovirus LTR) and also containing AR nucleotide sequences.

The host cell is used to produce the polypeptides of interest. Because the AR contains a membrane bound molecule, it is purified from the host cell membranes or the AR is utilized while anchored in the cell membrane, i.e., whole cells or membrane fractions of cells are used. Purification or enrichment of the ARs from such expression systems is accomplished using appropriate detergents and lipid micelles by methods well known to those skilled in the art.

In bacterial systems, a number of expression vectors may be advantageously selected depending upon the use intended for the gene product being expressed. For example, when a large quantity of such protein is produced for the generation of antibodies to ARs, vectors that direct the expression of high levels of protein products are desirable. One skilled in the art is able to generate such vector constructs and purify the proteins by a variety of methodologies including selective purification technologies such as fusion protein selective columns and antibody columns, and non-selective purification technologies.

In an insect protein expression system, the baculovirus A. californica nuclear polyhedrosis virus (AcNPV), is used as a vector to express foreign genes in S. frugiperda cells. In this case, AR nucleotide sequences are cloned into non-essential regions of the virus and placed under the control of an AcNPV promoter. The recombinant viruses are then used to infect cells in which the inserted gene is expressed and the protein is purified by one of many techniques known to one skilled in the art.

In mammalian host cells, a number of viral-based expression systems may be utilized. Utilization of these expression systems often requires the creation of specific initiation signals in the vectors for efficient translation of the inserted nucleotide sequences. This is particularly important if a portion of the AR gene is used which does not contain the endogenous initiation signal. The placement of this initiation signal, in frame with the coding region of the inserted nucleotide sequence, as well as the addition of transcription and translation enhancing elements and the purification of the recombinant protein, are achieved by one of many methodologies known to one skilled in the art. Also important in mammalian host cells is the selection of an appropriate cell type that is capable of the necessary posttranslational modifications of the recombinant protein. Such modifications, for example, cleavage, phosphorylation, glycosylation, etc., require the selection of the appropriate host cell that contains the modifying enzymes. Such host cells include, but are not limited to, CHO, HEK293, NIH3T3, COS, etc. and are known by those skilled in the art.

For long term, high expression of recombinant proteins, stable expression is preferred. For example, cell lines that stably express ARs may be engineered. One of skill in the art, following known methods such as electroporation, calcium phosphate transfection, or liposome-mediated transfection, can generate a cell line that stably expresses ARs. This is usually accomplished by transfecting cells using expression vectors that contain appropriate expression control elements (e.g., promoter sequences, enhancer sequences, transcriptional termination sequences, polyadenylation sites, translational start sites, etc.), a selectable marker, and the gene of interest. The selectable marker may either be contained within the same vector, as the gene of interest, or on a separate vector, which is co-transfected with the AR sequence-containing vector. The selectable marker in the expression vector may confer resistance to the selection and allows cells to stably integrate the vector into their chromosomes and to grow to form foci that in turn can be cloned and expanded into cell lines. Alternatively, the expression vector may allow selection of the cell expressing the selectable marker utilizing a physical attribute of the marker, i.e., expression of Green Fluorescent Protein (GFP) allows for selection of cells expressing the marker using fluorescence activated cell sorting (FACS) analysis.

One of skill in the art is able to select an appropriate cell type for transfection in order to allow for selection of cells into which the gene of interest has been successfully integrated. For example, where the selectable marker is herpes simplex virus thymidine kinase, hypoxanthine-guanine phosphoribosyltransferase or adenine phosphoribosyltransferase, the appropriate cell type would be tk-, hgprt- or aprt- cells, respectively. Or, normal cells can be used where the selectable marker is dhfr, gpt, neo or hygro that confer resistance to methotrexate, mycophenolic acid, G-418 or hygromycin, respectively. Such recombinant cell lines are useful for identification of candidate compounds that affect the AR activity.

IV. Preparation of AR Antibodies

Antibodies that selectively recognize one or more epitopes of an AR are also encompassed by the invention. Such antibodies include, e.g., polyclonal antibodies, monoclonal antibodies, chimeric antibodies, human antibodies, single chain antibodies, Fab fragments, F(ab′)₂ fragments, molecules produced using a Fab expression library, human antibodies (polyclonal or monoclonal) produced in transgenic mice and epitope binding fragments of any of the above. For therapeutic uses, chimeric or human antibodies are preferred; human antibodies are most preferred.

The antibodies can be utilized in conjunction with the compound screening schemes described herein for the evaluation of test compounds, e.g., for immobilization of AR polypeptides or such antibodies can be used in conjunction with gene therapy techniques to evaluate, for example, the expression of ARs either in cells or directly in patient tissues in which these genes have been introduced. In addition, antibodies of the present invention are useful in the treatment of skeletal muscle atrophy. Antibodies selective for the AR can be screened by the methods of the present invention to identify a subset of the antibodies that are AR agonists. In addition, anti-idiotype antibodies generated against antibodies specific for amylin or an amylin analog may be useful as AR agonists and like anti-AR antibodies may be screened for their ability to activate the AR by methods of the present invention.

For the production of antibodies, a variety of host animals may be immunized by injection with AR, amylin or an amylin analog, anti-amylin antibody, anti-amylin analog antibody, or immunogenic fragments thereof by methods well known in the art. For preparation of an anti-idiotype antibody the immunogen is an anti-amylin antibody or anti-amylin analog antibody. Production of anti-idiotype antibodies is described, for example, in U.S. Pat. No. 4,699,880, incorporated herein by reference. Suitable host animals include, but are not limited to, rabbits, mice, goats, sheep and horses. Immunization techniques are well known in the art. Polyclonal antibodies can be purified from the serum of the immunized animals, or monoclonal antibodies can be generated by methods that are well known in the art. These techniques include, but are not limited to, the well-known hybridoma techniques of Kohler and Milstein, human B-cell hybridoma techniques, and the EBV hybridoma technology. Monoclonal antibodies may be of any immunoglobulin class, including IgG, IgE, IgM, IgA, and IgD containing either kappa or lambda light chains.

Because of the immunogenicity of non-human antibodies in humans, chimeric antibodies are preferred to non-human antibodies when used for therapeutic treatment of human patients. Techniques of producing and using chimeric antibodies are known in the art, and are described in, for example, U.S. Pat. Nos. 5,807,715; 4,816,397; 4,816,567; 5,530,101; 5,585,089; 5,693,761; 5,693,762; 6,180,370; and 5,824,307, all incorporated herein by reference.

Completely human antibodies are particularly desirable for therapeutic treatment of human patients because they are less immunogenic than non-human antibodies or chimeric antibodies. Such antibodies can be produced using transgenic mice which are substantially incapable of expressing endogenous immunoglobulin heavy and light chain genes, but which can express human heavy and light chain genes. The transgenic mice are immunized in the normal fashion with a selected antigen, e.g., all or a portion of ARs. Monoclonal antibodies directed against the antigen are obtained using conventional hybridoma technology from these immunized transgenic mice. This technology is described in detail in U.S. Pat. Nos. 5,874,299; 5,877,397; 5,569,825; 5,661,016; 5,770,429; and 6,075,181, all incorporated herein by reference. As an alternative to obtaining human immunoglobulins directly from the culture of the hybridoma cells, the hybridoma cells can be used as a source of rearranged heavy chain and light chain loci for subsequent expression or genetic manipulation. Isolation of genes from such antibody-producing cells is straightforward since high levels of the appropriate mRNAs are available. The recovered rearranged loci can be manipulated as desired. For example, the constant region can be eliminated or exchanged for that of a different isotype or the variable regions can be linked to encode single chain Fv regions. Such techniques are described in WO 96/33735 and WO 96/34096, all incorporated herein by reference.

V. Selection of Test Compounds

Compounds that can be screened in accordance with the assays of the invention include but are not limited to, libraries of known compounds, including natural products, such as plant or animal extracts, synthetic chemicals, biologically active materials including proteins, peptides such as soluble peptides, including but not limited to members of random peptide libraries and combinatorial chemistry derived molecular library made of D- or L-configuration amino acids, phosphopeptides (including, but not limited to, members of random or partially degenerate, directed phosphopeptide libraries), antibodies (including, but not limited to, polyclonal, monoclonal, chimeric, human, anti-idiotypic or single chain antibodies, and Fab, F(ab′)₂ and Fab expression library fragments, and epitope-binding fragments thereof), organic and inorganic molecules.

In addition to the more traditional sources of test compounds, computer modeling and searching technologies permit the rational selection of test compounds by utilizing structural information from the ligand binding site of AR or from already identified agonists of ARs. Such rational selection of test compounds can decrease the number of test compounds that must be screened in order to identify a candidate therapeutic compound. ARs are GPCRs, and thus knowledge of the AR protein sequences allows for the generation of a model of its binding site that can be used to screen for potential ligands. This process can be accomplished in several manners well known in the art. Briefly, the most robust approach involves generating a sequence alignment of the AR sequences to a template (derived from the bacterio-rhodopsin or rhodopsin crystal structures or other GPCR model), conversion of the amino acid structures and refining the model by molecular mechanics and visual examination. If a strong sequence alignment cannot be obtained then a model may also be generated by building models of the hydrophobic helices. These are then fitted together by rotating and translating each helix relative to the others starting from the general layout of the known rhodopsin structures. Mutational data that point towards residue-residue contacts may also be used to position the helices relative to each other so that these contacts are achieved. During this process, docking of the known ligands into the binding site cavity within the helices may also be used to help position the helices by developing interactions that would stabilize the binding of the ligand. The model may be completed by refinement using molecular mechanics and loop building of the intracellular and extracellular loops using standard homology modeling techniques. General information regarding GPCR structure and modeling can be found in Schoneberg, T. et. al., Molecular and Cellular Endocrinology, 151:181-193 (1999), Flower, D., Biochimica et Biophysica Acta, 1422:207-234 (1999), and Sexton, P. M., Current Opinion in Drug Discovery and Development, 2(5):440-448 (1999).

Once the model is completed, it can be used in conjunction with one of several existing computer programs to narrow the number of compounds to be screened by the screening methods of the present invention. The most general of these is the DOCK program (UCSF Molecular Design Institute, 533 Parnassus Ave, U-64, Box 0446, San Francisco, Calif. 94143-0446). In several of its variants it can screen databases of commercial and/or proprietary compounds for steric fit and rough electrostatic complementarity to the binding site. It has frequently been found that molecules that score well within DOCK have a better chance of being ligands. Another program that can be used is FLEXX (Tripos Inc., 1699 South Hanley Rd., St. Louis, Mo.). This program, being significantly slower, is usually restricted to searches through smaller databases of compounds. The scoring scheme within FLEXX is more detailed and usually gives a better estimate of binding ability than does DOCK. FLEXX is best used to confirm DOCK suggestions, or to examine libraries of compounds that are generated combinatorially from known ligands or templates.

VI. Screening Assays to Identify Candidate Compounds for the Regulation of Skeletal Muscle Mass or Function

The finding that ARs plays a role in regulating skeletal muscle atrophy enables various methods of screening one or more test compounds to identify candidate compounds that ultimately may be used for prophylactic or therapeutic treatment of skeletal muscle atrophy. This invention provides methods for screening test compounds for their ability to bind to ARs, activate ARs, prolong or augment the agonist-induced activation of ARs or of a AR signal transduction pathway or increase expression of AR or amylin genes.

For screening compounds which ultimately will be used to regulate skeletal muscle mass or function through ARs in humans, it is preferred that the initial in vitro screen be carried out using either AR1 or AR2 with an amino acid sequence that is greater than 78% identical to SEQ ID NO: 62 and more preferably greater than 90% identical to SEQ ID NO: 62. More preferably the test compounds will be screened against a human, mouse or rat AR, with the most preferable being human. For screening compounds which ultimately will be used to regulate skeletal muscle mass or function through ARs in a non-human species it is preferable to use the AR from the species in which treatment is contemplated.

The methods of the present invention are amenable to high throughput applications; however, the use of as few as one test compound in the method is encompassed by the term “screening”. Test compounds which bind to ARs, activate ARs, prolong or augment the agonist-induced activation of ARs or of an AR signal transduction pathway, or increase expression of AR or amylin genes, as determined by a method of the present invention, are referred to herein as “candidate compounds.” Such candidate compounds can be used to regulate skeletal muscle mass or function. However, more typically, this first level of in vitro screen provides a means by which to select a narrower range of compounds, i.e., the candidate compounds, which merit further investigation in additional levels of screening. The skilled artisan will recognize that a utility of the present invention is to identify, from a group of one or more test compounds, a subset of compounds which merit further investigation. One of skill in the art will also recognize that the assays of the present invention are useful in ranking the probable usefulness of a particular candidate compound relative to other candidate compounds. For instance, a candidate compound which activates AR at 1000 nM (but not at 10 nM) is of less interest than one that activates AR at 10 nM. Using such information the skilled artisan may select a subset of the candidate compounds, identified in the first level of screening, for further investigation. By the way of example only, compounds which activate AR at concentrations of less than 200 nM might be further tested in an animal model of skeletal muscle atrophy, whereas those above that threshold would not be further tested. The skilled artisan will also recognize that, depending on how the group of test compounds is selected, and how the positive test compounds are selected, only a certain proportion of test compounds will be identified as candidate compounds, and that this proportion may be very small.

The assay systems described below may be formulated into kits comprising an AR or cells expressing an AR which can be packaged in a variety of containers, e.g., vials, tubes microtitre well plates, bottles and the like. Other reagents can be included in separate containers and provided with the kit, e.g., positive control samples, negative control samples, buffers and cell culture media.

In one embodiment, the invention provides a method for screening one or more test compounds to identify candidate compounds that bind to an AR. Methods of determining binding of a compound to a receptor are well known in the art. Typically, the assays include the steps of incubating a source of an AR with a labeled compound, known to bind to the receptor, in the presence or absence of a test compound and determining the amount of bound labeled compound. The source of the AR may either be cells expressing an AR or some form of isolated AR, as described herein. The labeled compound can be amylin or any amylin analog labeled such that it can be measured, preferably quantitatively (e.g., ¹²⁵I-labeled, europium labeled, fluorescein labeled, GFP labeled, ³⁵S-methionine labeled). Such methods of labeling are well known in the art. Test compounds that bind to an AR cause a reduction in the amount of labeled ligand bound to the receptor, thereby reducing the signal level compared to that from control samples (absence of test compound). Variations of this technique have been described in which receptor binding in the presence and absence of G-protein uncoupling agents can discriminate agonists from antagonists (e.g., binding in the absence and presence of a guanine nucleotide analog i.e., GpppNHp). See Keen, M., Radioligand Binding Methods for Membrane Preparations and Intact cells in Receptor Signal Transduction Protocols, R. A. J. Challis, (ed), Humana Press Inc., Totoway N.J. (1997).

Because it is desirable to discriminate between compounds that bind specifically to an AR as compared to other related GPCRs, the assays described above should be conducted using a cell, or membrane from a cell, which expresses only the AR or interest or the assays can be conducted with a recombinant source of AR. Cells expressing additional GPCRs that may interact with the AR ligands may be modified using homologous recombination to inactivate or otherwise disable the GPCR gene. Alternatively, if the source of AR contains additional GPCRs, the background signal produced by the receptor that is not of interest must be subtracted from the signal obtained in the assay. The background response can be determined by a number of methods, including elimination of the signal from the GPCR that is not of interest by use of antisense, antibodies or selective antagonists.

In another embodiment, the invention provides methods for screening test compounds to identify candidate compounds that activate ARs. Typically, the assays are cell-based; however, cell-free assays are known which are able to differentiate agonist and antagonist binding as described above. Cell-based assays include the steps of contacting cells which express an AR with a test compound or control and measuring activation of the AR by measuring the expression or activity of components of the AR signal transduction pathways.

As described in the background section above, ARs appear to couple through several different pathways, including the G_{αs} signal transduction pathway, depending upon the cell type. It is thought that agonist activation of an AR allows the receptor to signal via any of these pathways, provided that the necessary pathway components are present in the particular cell type. Thus, to screen for AR activation, an assay can use any of the signal transduction pathways as the readout even if the relevant cell type for treatment, in vivo, couples AR to skeletal muscle atrophy via a different pathway. One of ordinary skill in the art would recognize that a screening assay would be effective for identifying useful AR agonists independent of the pathway by which receptor activation was measured. Assays for measuring activation of these signaling pathways are known in the art.

For example, after contact with the test compound, lysates of the cells can be prepared and assayed for induction of cAMP. cAMP is induced in response to G_{αs} activation. Because G_{αs} is activated by receptors other than ARs and because a test compound may be exerting its effect through ARs by another mechanism, two control comparisons are relevant for determining whether a text compound increases levels of cAMP via activation of an AR. One control compares the cAMP level of cells contacted with a test compound and the cAMP level of cells contacted with a control compound (i.e., the vehicle in which the test compound is dissolved). If the test compound increases cAMP levels relative to the control compound this indicates that the test compound is increasing cAMP by some mechanism. The other control compares the cAMP levels of an AR expressing cell line and a cell line that is essentially the same except that it does not express the AR, where both of the cell lines have been treated with test compound. If the test compound elevates cAMP levels in the AR expressing cell line relative to the cell line that does not express ARs, this is an indication that the test compound elevates cAMP via activation of the AR.

In a specific embodiment of the invention, cAMP induction is measured with the use of DNA constructs containing the cAMP responsive element linked to any of a variety of reporter genes can be introduced into cells expressing ARs. Such reporter genes include, but are not limited to, chloramphenicol acetyltransferase (CAT), luciferase, glucuronide synthetase, growth hormone, fluorescent proteins (e.g., Green Fluorescent Protein), or alkaline phosphatase. Following exposure of the cells to the test compound, the level of reporter gene expression can be quantitated to determine the test compound's ability to increase cAMP levels and thus determine a test compounds ability to activate the AR.

The cells useful in this assay are the same as for the AR binding assay described above, except that cells utilized in the activation assays preferably express a functional receptor which gives a statistically significant response to amylin or one or more amylin analog. In addition to using cells expressing full length ARs, cells can be engineered which express ARs containing the ligand binding domain of the receptor coupled to, or physically modified to contain, reporter elements or to interact with signaling proteins. For example, a wild type AR or AR fragment can be fused to a G-protein resulting in activation of the fused G-protein upon agonist binding to the AR portion of the fusion protein. (Siefert, R. et al., Trends Pharmacol. Sci. 20: 383-389 (1999)). The cells should also preferably possess a number of characteristics, depending on the readout, to maximize the inductive response by amylin or the amylin analog, for example, for detecting a strong induction of a CRE reporter gene; (a) a low natural level of cAMP; (b) G proteins capable of interacting with ARs; (c) a high level of adenylyl cyclase; (d) a high level of protein kinase A; (e) a low level of phosphodiesterases; and (f) a high level of cAMP response element binding protein would be advantageous. To increase the response to amylin or an amylin analog, host cells could be engineered to express a greater amount of favorable factors or a lesser amount of unfavorable factors. In addition, alternative pathways for induction of the CRE reporter could be eliminated to reduce basal levels.

In some instances, G protein-coupled receptor responses subside, or become desensitized, after prolonged exposure to an agonist. Another embodiment of the invention provides methods for identifying compounds that prolong or augment the agonist-induced activation of ARs, or the AR signal transduction pathway, in response to an AR agonist. Such compounds may be used, for example, in conjunction with an AR agonist for the treatment of skeletal muscle atrophy. Typically the method uses a cell based assay comprising in any order or concurrently (i) contacting the cells with a test compound; (ii) treating cells expressing functional AR with an AR agonist at a concentration of agonist and for a period of agonist-receptor exposure sufficient to allow desensitization of the receptor; followed by (iii) determining the level of activation of the AR. One of skill in the art will recognize that several mechanisms contribute to receptor desensitization including, but not limited to, receptor phosphorylation, receptor internalization or degradation and AR signal transduction pathway down-modulation. One of skill in the art can determine the appropriate time (i.e., before, during or after agonist treatment) for contacting the cells with the test compounds depending upon which mechanism of desensitization is targeted. For example, contacting the cells with test compounds following agonist treatment, can detect test compounds which block receptor desensitization that occurs as a result of phosphorylation of the receptor.

In another embodiment, the invention provides a method of screening one or more test compound to identify candidate compounds that regulate transcription from an AR gene or regulate AR expression. Candidate compounds that regulate transcriptional activity of AR genes may be identified using a reporter gene operably associated with an AR regulatory region (reporter gene construct). Such methods are known in the art. In one such method, the reporter gene construct is contacted with a test compound in the presence of a source of cellular factors and the level of reporter gene expression is determined. A test compound that causes an increase in the level of expression, compared to a control sample, is indicative of a candidate compound that increases transcription of an AR gene. To provide the cellular factors required for in vitro or in vivo transcription, appropriate cells or cell extracts are prepared from any cell type that normally expresses an AR.

Candidate compounds that regulate an AR expression can also be identified in a method wherein a cell is contacted with a test compound and the expression of an AR is determined. The level of expression of an AR in the presence of the test compound is compared with the level of expression in the absence of the test compound. Test compounds that increase the expression of an AR are identified as candidate compounds for increasing muscle mass or muscle function. Such a method detects candidate compounds which increase the transcription or translation of an AR or which increase the stability of the mRNA or AR protein.

In another embodiment, this invention provides methods for screening one or more test compounds to identify candidate compounds that regulate the expression of the amylin or an amylin analog. Such assays are performed essentially as described above for the assays to identify candidate compounds that regulate expression of ARs with the following modifications. To identify candidate compound that regulate transcription from the amylin gene or an amylin analog gene, the reporter gene is operably associated with the regulatory region of the amylin gene or amylin analog gene of interest and the source of cellular factors should be from a cell type that expresses the gene of interest.

VII. Screening of Candidate Compounds using Models of Skeletal Muscle Atrophy

Candidate compounds selected from one or more test compounds by an in vitro assay, as described above, can be further tested for their ability to regulate skeletal muscle mass or function in model systems of skeletal muscle atrophy and/or hypertrophy. Such models of skeletal muscle atrophy or hypertrophy include both in vitro cell culture models and in vivo animal models of skeletal muscle atrophy. Such additional levels of screening are useful to further narrow the range of candidate compounds that merit additional investigation, e.g., clinical trials.

Cell Culture Models of Muscle Atrophy

In vitro models of skeletal muscle atrophy are known in the art. Such models are described, for example, in Vandenburgh, H. H., In Vitro 24:609-619 (1988), Vandenburgh, H. H. et al., J of Biomechanics, 24 Suppl 1:91-99 (1991), Vandenburgh, H. H et al., In Vitro Cell. Dev. Biol., 24(3):166-174 (1988), Chromiak, J. A., et al., In Vitro Cell. Dev. Biol. Anim., 34(9):694-703(1998), Shansky, J., et al., In Vitro Cell. Dev. Biol. Anim., 33(9):659-661 (1997), Perrone, C. E. et al., J. Biol. Chem. 270(5):2099-2106 (1995), Chromiac, J. A. and Vandenburgh, H. H., J. Cell. Physiol. 159(3):407-414 (1994), and Vandenburgh, H. H. and Karlisch, P., In Vitro Cell. Dev. Biol. 25(7):607-616 (1989). Such models are useful, but not required, following the in vitro screening described above in order to further narrow the range of candidate compounds that merit testing in an animal model. Cell culture models are treated with candidate compounds and the response of the model to the treatment is measured by assessing changes in muscle markers such as: muscle protein synthesis or degradation, changes in skeletal muscle mass or contractile function. Those compounds that induce significant changes in the muscle markers are typically screened further in an animal model of skeletal muscle atrophy.

Animal Models of Skeletal Muscle Atrophy

The candidate compounds are administered to non-human animals and the response of the animals is monitored, for example, by assessing changes in markers of atrophy or hypertrophy such as: skeletal muscle mass, skeletal muscle function, muscle or myofiber cross-sectional area, contractile protein content, non-contractile protein content or a biochemical or genetic marker that correlates with skeletal muscle mass or function changes. Candidate compounds that induce skeletal muscle hypertrophy or prevent any aspect of skeletal muscle atrophy should be considered as prospective therapeutic candidates for treatment of human skeletal muscle atrophy, and are referred to herein as candidate therapeutic compounds. In addition to assessing the ability of a candidate compound to regulate skeletal muscle atrophy, undesirable side effects such as toxicity may also be detected in such a screen. The absence of unacceptably high levels of side effects may be used as a further criterion for the selection of candidate therapeutic compounds.

A variety of animal models for skeletal muscle atrophy are known in the art, such as those described in the following references: Herbison, G. J., et al. Arch. Phys. Med. Rehabil. 60:401-404 (1979), Appell, H-J. Sports Medicine 10:42-58 (1990), Hasselgren, P-O. and Fischer, J. E. World J. Surg. 22:203-208 (1998), Agbenyega, E. T. and Wareham, A. C. Comp. Biochem. Physiol. 102A:141-145 (1992), Thomason, D. B. and Booth, F. W. J. Appl. Physiol. 68:1-12 (1990), Fitts, R. H., et al. J. Appl. Physiol. 60:1946-1953 (1986), Bramanti, P., et al. Int. J. Anat. Embryol. 103:45-64 (1998), Cartee, G. D. J. Gerontol. A Biol. Sci. Med. Sci. 50:137-141 (1995), Cork, L. C., et al. Prog. Clin. Biol. Res. 229:241-269 (1987), Booth, F. W. and Gollnick, P. D. Med. Sci. Sports Exerc. 15:415-420 (1983), Bloomfield, S. A. Med. Sci. Sports Exerc. 29:197-206 (1997). Preferred animals for these models are mice and rats. These models include, for example, models of disuse-induced atrophy such as casting or otherwise immobilizing limbs, hind limb suspension, complete animal immobilization, and reduced gravity situations. Models of nerve damage induced atrophy include, for example, nerve crush, removal of sections of nerves that innervate specific muscles, toxin application to nerves and infection of nerves with viral, bacterial or eukaryotic infectious agents. Models of glucocorticoid-induced atrophy include application of atrophy-inducing doses of exogenous glucocorticoid to animals, and stimulation of endogenous corticosteroid production, for example, by application of hormones that activate the hypothalamus-pituitary-adrenal (HPA) axis. Models of sepsis-induced atrophy include, for example, inoculation with sepsis-inducing organisms such as bacteria, treatment of the animal with immune-activating compounds such as bacterial cell wall extract or endotoxin, and puncture of intestinal walls. Models of cachexia-induced atrophy include, for example, inoculation of an animal with tumorigenic cells with cachexia forming potential, infection of an animal with infectious agents (such as viruses which cause AIDS) which result in cachexia and treatment of an animal with hormones or cytokines such as CNTF, TNF, IL-6, IL-1, etc. which induce cachexia. Models of heart failure-induced atrophy include the manipulation of an animal so that heart failure occurs with concomitant skeletal muscle atrophy. Neurodegenerative disease-induced atrophy models include autoimmune animal models such as those resulting from immunization of an animal with neuronal components. Muscular dystrophy-induced models of atrophy include natural or man-made genetically-induced models of muscular dystrophy such as the mutation of the dystrophin gene which occurs in the Mdx mouse.

Animal models of skeletal muscle hypertrophy include, for example, models of increased limb muscle use due to inactivation of the opposing limb, reweighting following a disuse atrophy inducing event, reutilization of a muscle which atrophied because of transient nerve damage, increased use of selective muscles due to inactivation of a synergistic muscle (e.g., compensatory hypertrophy), increased muscle utilization due to increased load placed on the muscle and hypertrophy resulting from removal of the glucocorticoid after glucocorticoid-induced atrophy. Preferred animal atrophy models include the sciatic nerve denervation atrophy model, glucocorticoid-induced atrophy model, and the leg casting disuse atrophy model that are described in further detail below.

The sciatic nerve denervation atrophy model involves anesthetizing the animal followed by the surgical removal of a short segment of either the right or left sciatic nerve, e.g., in mice the sciatic nerve is isolated approximately at the midpoint along the femur and a 3-5 mm segment is removed. This denervates the lower hind limb musculature resulting in atrophy of these muscles. Typically, innervation to the biceps femoris is left intact to provide satisfactory motion of the knee for virtually normal ambulation. Typically, in untreated animals, muscle mass of the denervated muscles is reduced 30-50% ten days following denervation. Following denervation, test compounds are administered e.g., by injection or by continuous infusion, e.g., via implantation of an osmotic minipump (e.g., Alzet, Palo Alto, Calif.), to determine their effect on denervation induced skeletal muscle atrophy. At various times following denervation, the animals are euthanized and lower leg muscles are dissected rapidly from both the denervated and nondenervated legs, the muscles, cleaned of tendons and connective tissue, are weighed. The extent of atrophy in the affected muscles is analyzed, for example, by measuring muscle mass, muscle cross-sectional area, myofiber cross-sectional area or contractile protein content.

The glucocorticoid-induced atrophy model involves the administration of a glucocorticoid to the test animal, e.g., 1.2 mg/kg/day of dexamethasone in the drinking water. Typically, in untreated animals, skeletal muscle mass is reduced 30-50% following ten days of dexamethasone administration. Concomitantly with, or following glucocorticoid administration, test compounds are administered e.g., by injection or by continuous infusion to determine their effect on glucocorticoid-induced skeletal muscle atrophy. At various times following glucocorticoid administration, the extent of atrophy in the affected muscles is analyzed as described above for the denervation model.

The leg casting disuse atrophy model involves casting one hind leg of an animal from the knee down through the foot. Typically, muscle mass is reduced 20-40% after ten days of casting. Following casting, test compounds are administered by injection or by continuous infusion via implantation of an osmotic minipump (e.g., Alzet, Palo Alto, Calif.) to determine their effect on leg casting induced skeletal muscle atrophy. At various times following leg casting, the extent of atrophy in the affected muscles is analyzed as described above for the denervation model.

One of skill in the art would recognize that in screening for compounds for human use, because there are differences between the human ARs and the ARs from other animal species, there may be some false positive or negative results which arise when the screen is carried out using non-human ARs. Thus, it is preferable to do the initial in vitro screen using human ARs. In certain circumstances, identified candidate compounds may be active toward only the human receptor and not toward a non-human receptor. In such circumstances, it may still be desirable to determine whether these candidate compounds are able to regulate skeletal muscle mass or function in a second level of screening. Because these candidates do not activate non-human ARs, a standard in vivo screen with non-human animal is not advised. In such circumstances the second level of screening for these candidates may be performed in transgenic animals that express human ARs.

Animals of any species, especially mammals, including, but not limited to, mice, rats, rabbits, guinea pigs, pigs, goats, dogs and non-human primates may be used to generate AR transgenic animals. Mice and rats are preferred, mice are most preferred. A variety of techniques are known in the art and may be used to introduce the human AR transgenes into animals to produce the founder lines of transgenic animals. Such techniques include, but are not limited to, pronuclear microinjection, retrovirus-mediated gene transfer into germ lines, gene targeting in embryonic stem cells, electroporation of embryos and sperm-mediated gene transfer.

VIII. Gene Therapy Methods for the Treatment of Skeletal Muscle Atrophy

The overall activity of ARs can be increased by overexpressing the genes for ARs (to increase expression of ARs) or a constitutively active AR in the appropriate tissue. Amylin levels can be increased, in vivo, by likewise overexpressing an amylin gene. Overexpression of these genes will increase the total cellular AR activity, thus, regulating skeletal muscle atrophy. The gene or genes of interest are inserted into a vector suitable for expression in the subject. These vectors include, but are not limited to, adenovirus, adenovirus associated virus, retrovirus and herpes virus vectors in addition to other particles that introduced DNA into cells (e.g., liposome, gold particles, etc.) or by direct injection of the DNA expression vector, containing the gene of interest, into human tissue (e.g., muscle).

IX. Pharmaceutical Formulations and Methods for Use

Candidate compounds or candidate therapeutic compounds identified by screening methods described herein, can be administered to individuals to treat skeletal muscle atrophy, or to induce skeletal muscle hypertrophy. To this end, the present invention encompasses methods and compositions for modulating skeletal muscle atrophy, including, but not limited to, skeletal muscle atrophy induced by disuse due to surgery, bed rest, broken bones; denervation/nerve damage due to spinal cord injury; autoimmune disease; infectious disease; glucocorticoid use for unrelated conditions; sepsis due to infection or other causes; nutrient limitation due to illness or starvation; cancer cachexia; chronic inflammation; AIDS cachexia; COPD; congestive heart failure; sarcopenia and genetic disorders; e.g., muscular dystrophies, neurodegenerative diseases. Agonists of ARs can be used to inhibit skeletal muscle atrophy. It is not necessary that effective compounds demonstrate absolute specificity for ARs. It is contemplated that specific antagonist of other affected receptors can be co-administered with an effective, but nonspecific, agonist. Alternately, this lack of specificity may be addressed by modulation of dose alone, or the dosing regimen.

The candidate compounds or candidate therapeutic compounds identified by the screening methods of the present invention may be administered in conjunction with compounds which prolong or augment the activation of ARs or of AR signal transduction pathways. These may be known compounds, for example, theophylline, or these compounds may be identified by the screening methods of this invention to prolong or augment the activation of ARs or of AR signal transduction pathways.

Dose Determinations

Safety and therapeutic efficacy of compounds that agonize ARs can be determined by standard procedures using either in vitro or in vivo technologies. Compounds that exhibit large therapeutic indices are preferred, although compounds with lower therapeutic indices are useful if the level of side effects is acceptable. The data obtained from the in vitro and in vivo toxicological and pharmacological techniques can be used to formulate the human range of doses that may be useful. The preferred dose lies in the range in which the circulating concentration of the compound is therapeutically maximal with acceptable safety. The circulating concentration of the compound may vary depending on the dose form, time after dosing, route of administration, etc. Doses outside this range are also useful provided the side effects are acceptable. Such matters as age and weight of the patient, and the like, can be used to determine such matters in the conventional manner. Pharmacogenetic approaches may be useful in optimizing compound selection, doses and dosing regimen in clinical populations.

Formulation and Use

Pharmaceutical compositions for use in the modulation of skeletal muscle atrophy in accordance with the present invention may be formulated using conventional methodologies using pharmaceutically acceptable carriers and excipients. The compositions of this invention are preferably provided in unit dosage form. As used herein, a “unit dosage form” is a composition of this invention containing an amount of an AR agonist that is suitable for administration to an animal, preferably a mammal, more preferably a human subject, in a single dose, according to good medical practice. Pharmaceutical compositions may be formulated for delivery by, for example, intranasal, transdermal, inhalation, parenteral, cutaneous, oral or rectal administration. For oral administration, the pharmaceutical composition may take the form of tablets or capsules containing the pharmacologically active compound and additives including, but not limited to, binding agents, fillers, lubricants, disintegrants, or wetting agents. The tablets may be coated. Liquid preparations for oral administration include, but are not limited to, syrups, suspensions or dry products which are reconstituted with liquid vehicle before use, containing the pharmacologically active compound and additives including, but not limited to, suspending agents, emulsifying agents, non-aqueous vehicles, preservatives, buffer salts, flavoring, coloring, sweetening agents, etc. Pharmaceutical compositions for oral administration may be formulated for controlled release of the pharmacologically active compounds either in the mouth, stomach or intestinal tract.

For inhalation administration, the compounds for use according to the present invention may be delivered by, but not limited to, the following forms: liquid, powder, gel or in the form of an aerosol spray utilizing either pressurized or non-pressurized propellants in either premeasured or non-premeasured doses. The pharmacologically active compound may be formulated with appropriate fillers, vehicles, preservatives, buffers, etc. For parenteral administration, the pharmacologically active compound may be formulated with acceptable physiological carriers, preservatives, etc. and be prepared as suspensions, solutions, emulsion, powders ready for constitution, etc. for either bolus injection or infusion. Doses of these compounds may be administered by a variety of technologies including hypodermic needles, high pressure devices, etc. For rectal administration, the pharmacologically active compound may be formulated with acceptable physiological carriers, preservatives, etc. for delivery as suppositories, enemas, etc. For cutaneous administration, the pharmacologically active compound may be formulated with acceptable physiological carriers including lotions, emollients, etc. or incorporated into a patch type device. For long term administration, the pharmacologically active compound and appropriate additives such as, but limited to, polymers, hydrophobic materials, resins, etc. may be formulated as a depot preparation for either injection or implantation at multiple sites including but not limited to intramuscular and subcutaneous locations. In addition, the pharmacologically active compound may be administered by a dispensing device.

Monitoring of Effects During Clinical Trials

Monitoring the influence of compounds (e.g., drugs) on the expression or activity of ARs can be employed not only in basic drug screening, but also in clinical trials. For example, the effectiveness of a compound determined by a screening assay to increase AR receptor activity or AR receptor expression can be assessed in clinical trials of patients with, or at risk for, skeletal muscle atrophy. At various times following administration of the test compound or placebo, the effect of the compound on the patient can be determined, for example, by observing the change in skeletal muscle mass, skeletal muscle function, biochemical markers of muscle breakdown or quality of life measures. Methods of measuring skeletal muscle mass in human subjects are known in the art and include, for example: measuring the girth of a limb; measuring muscle thickness with for instance, computer tomography, MRI or supersonics; or muscle biopsy to examine morphological and biochemical parameters (e.g., cross-section fiber area, fiber diameter or enzyme activities). Furthermore, because skeletal muscle mass is correlated with skeletal muscle function, muscle function can be used as a surrogate marker of mass and muscle mass changes can be assessed using functional measurements, e.g., strength, the force of a group of synergist muscles, or contraction characteristics found in electromyographic recordings. In addition, muscle protein loss as a result of muscle atrophy can be measured by quantitating levels of amino acids or amino acids derivatives, i.e., 3-methyl histidine, in the urine or blood of a subject. For a review of such methods see Appell, Sports Med. 10:42-58 (1990). Quality of life measures include, but are not limited to, the ease of getting out of a chair, number of steps taken before tiring or ability to climb stairs.

### EXAMPLES

### Example 1

Construction of Vectors for Human AR (Human Calcitonin Receptor Associated with RAMP) Receptor Expression.

The human calcitonin receptor (hCR) DNA sequence, Accession No. X69920 (SEQ ID NO: 3), is retrieved and two oligonucleotides including one containing the 5′ end of the gene beginning at the initiation codon (5′ oligonucleotide) and one containing the 3′ end of the gene containing the stop codon (3′ oligonucleotide) are synthesized. Using the above 5′ and 3′ oligonucleotides, the hAR cDNA is amplified by PCR from the human skeletal muscle cDNA library available commercially using a PCR kit. The hCR gene PCR product is purified and cloned into the pIRESneo vector (Clonetech Inc., Palo Alto, Calif., USA) by commercially available PCR cloning kit according to the manufacturer's recommendations. pIRESneo/hCR is then used to transform competent E. coli cells. Plasmid DNA is isolated and insert from at least one clone is sequenced to ensure that the hCR sequence is correct. HEK293 cells containing a stably integrated Mercury CRE-LUC plasmid (Clonetech Inc., Palo Alto, Calif., USA) are transfected with purified pIRESneo/hCR DNA. Cells stably transfected with pIRESneo/hAR DNA are selected by culturing the cells in G418. The stably transfected cells (HEK293/CRE-LUC/pIRESneo/hCR cells) are propagated in DMEM (Life Technologies, Rockville, Md.) containing 10% fetal bovine serum at 37° C. in a 5% carbon dioxide/95% air atmosphere. The clones are then characterized to ensure they have the correct Receptor Activity Modifying Protein (RAMP) profile to ensure high affinity amylin receptors (RAMP1 or RAMP3). If the correct RAMPs are not expressed in the cell line then the correct RAMP is expressed in a RAMP negative cell line along with the human calcitonin receptor so that high affinity hAR1 and hAR2 can be expressed. The clones are characterized for both amylin binding and CRE-LUC activation following exposure to amylin as described in Example 2 and Example 3. Cells expressing the hAR receptor at an appropriate level and which are appropriately coupled to the CRE-LUC reporter system are then utilized for further analysis.

### Example 2

Receptor Binding Assays

Receptor binding analysis of compounds is performed in whole cells by plating the HEK293/CRE-LUC/pIRESneo/hCR cells from Example 1 in a 96 well polylysine coated plate. Cells are seeded in DMEM medium containing 10% fetal bovine serum at 37° C. in a 5% CO₂ and incubated overnight. The culture medium is removed and the appropriate amount of amylin covalently labeled with Europium (Eu-amylin) in MEM+10% Seablock (Clonetech Inc., Palo Alto, Calif., USA) is added. The cells are incubated with the Eu-amylin for 90 minutes at room temperature then washed 4 times with phosphate buffered saline lacking magnesium and calcium. Following the final wash, enhancement solution is added and the plate is read on a plate reader. For saturation binding analysis, log doses of Eu-amylin ranging from 10⁻¹² to 10⁻³ M are added to the cells and binding analyzed both in the absence and the presence of a saturating concentration of unlabeled amylin for evaluation of non-specific binding. For competitive binding, a concentration of Eu-amylin is added which is half maximal, in terms of binding, in addition to varying concentrations of the compound of interest.

### Example 3

Receptor Activation Assay

Receptor activation analysis is performed by seeding the HEK293/CRE-LUC/pIRESneo/hCR cells of Example 1 into Packard View Plate-96 (Packard Inc., CA). Cells are seeded in DMEM containing 10% fetal bovine serum at 37° C. in a 5% CO₂ and incubated overnight. The medium is then removed and replaced with DMEM containing 0.01% bovine albumin fraction V containing the compound of interest. The cells are further incubated for four hours at 37° C. after which the medium is removed and the cells are washed twice with Hanks Balanced Salt Solution (HBSS). Lysis Reagent is then added to the washed cells and incubated for 20 minutes at 37° C. The cells are then placed at −80° C. for 20 minutes followed by a 20 minute incubation at 37° C. After this incubation, Luciferase Assay Buffer and Luciferase Assay Substrate (Promega Inc., Madison, Wis.) are added to the cell lysates and luciferase activity quantitated using a luminometer. Relative activity of a compound is evaluated by comparing the increase following exposure to compound to the level of luciferase in HEK cells that contain the CRE-LUC construct without the hCR following exposure to compound. Specificity of response is also checked by evaluating luciferase response of hCR/CRE-LUC HEK cells to compound in the presence and absence of a 10-fold excess of hAR antagonist.

### Example 4

Screen to Identify Candidate Compounds that Prolong or Augment the Activation of AR and/or an AR Receptor Signal Transduction Pathway.

Identification of compounds that prolong or augment the agonist-induced activation of the AR or of an AR signal transduction pathway, involves a variation of the Receptor Activation Assay described in Example 3. Specifically, this assay is performed by seeding the HEK293/CRE-LUC/pIRESneo/hCR receptor cells into Packard View Plate-96 (Packard Inc., CA). Cells are seeded in DMEM medium containing 10% fetal bovine serum and saturating amounts of amylin at 37° C. in a 5% CO₂ and incubated for 48 hours. The medium is then removed and replaced with DMEM containing 0.01% bovine albumin fraction V and amylin in addition to the compound of interest. The cells are then incubated for four hours at 37° C. in a 5% CO₂ after which the medium is removed and the cells are washed twice with HBSS. Cells are processed as in Example 3 and Luciferase Assay Buffer and Luciferase Assay Substrate are added to the cell lysates and luciferase activity is quantitated using a luminometer. Test compounds which stimulate fluorescence significantly above the levels of control untreated cells, after correction for variations in cell density, are considered candidate compounds for regulating skeletal muscle mass or function. The compounds of most interest are those that induce relatively higher levels of fluorescence.

### Example 5

Screen to Identify Candidate Compounds Specific for ARs.

Compounds that activate ARs are identified as in Example 3. To select those compounds that show selectivity for AR1 and AR2 over lower affinity ARs, these compounds also are screened against lower affinity ARs. HEK293/CRE-LUC/pIRESneo/human lower affinity ARs cells are generated essentially as described in Example 1 except that human calcitonin receptor like receptor (hCRLR) DNA sequence, Accession No. X72304, is used for the initial PCR amplification. The human calcitonin receptor is coexpressed in a cell line with RAMP2 or the human calcitonin receptor like receptor is coexpressed in a cell line with either RAMP1, RAMP2 or RAMP3 to generate the panel of lower affinity amylin receptors. To determine how active the compounds are against lower affinity ARs, an activation assay is performed essentially as described in Example 3 except that HEK293/CRE-LUC/pIRESneo/human lower affinity AR cells are used to seed the plates. The amount of fluorescence stimulated by the compound in AR expressing cells is compared with the amount of fluorescence stimulated by the compound in lower affinity AR expressing cells. Those compounds which demonstrate a 10-fold better response (on a molar basis) in AR expressing cells than in lower affinity AR expressing cells are then checked further for specificity of response to eliminate differences due to clonal variation. HEK293/CRE-LUC/pIRESneo/hCR cells are assayed with the compound in the presence or absence of a 10-fold excess of the AR antagonist, acetyl-amylin (8-37). Those compounds that show greater than 10-fold selectivity for ARs and whose activity is inhibited by acetyl-amylin (8-37) are selected as candidate compounds.

### Example 6

Screens to Identify Candidate Compounds that Increase hCR, RAMP, or Amylin Expression

The sequence containing the promoter region of either the human calcitonin receptor gene, RAMP1 or RAMP3 genes, or amylin gene; beginning far enough upstream of the transcriptional initiation site to contain all the regulatory elements necessary for physiological expression of the gene in the appropriate tissue is retrieved from the human genome database. Two oligonucleotides, one containing the 5′ end of the promoter region (5′ oligonucleotide) and one containing the 3′ end of the promoter region including the transcriptional start site (3′ oligonucleotide) are synthesized. The 5′ and 3′ oligonucleotides are used for PCR amplification of the gene regulatory region from human DNA using a PCR kit. The gene regulatory region PCR product is purified and cloned in a suitable commercially available vector. Competent E. coli cells are transformed, and plasmid DNA is isolated, and the construct containing the gene regulatory region is analyzed by DNA sequencing to ensure construct correctness and integrity. Purified plasmid DNA containing the gene regulatory region is then transfected into the HEK293 cells, clones are selected using G418, isolated and propagated in DMEM containing 10% FBS and G418 at 37° C. in a 5% CO₂. G418 resistant clones are characterized by Southern blotting to ensure that they contain the gene regulatory promoter sequence; in addition activation of the gene regulatory region is analyzed using an appropriate stimulating agent. Cells expressing the appropriate gene regulatory region-ECFP at an appropriate level are then used in assays designed to evaluate compounds that can modulate the activity of the gene regulatory region as follows. The regulatory region activation analysis is performed by seeding the gene regulatory region-ECFP containing HEK293 cells at an appropriate density into black with clear bottom 96 well microtiter plates and allowed to grow overnight. The following day, the medium is removed and the test compound is added in fresh growth medium. The cells are incubated for 16 hours at 37° C. in a 5% CO₂ followed by measurement of fluorescence (excitation at 433 (453) nm followed by detecting emission at 475 (501) nm using a fluorometer. Test compounds which stimulate fluorescence significantly above the levels of control untreated cells are considered candidate compounds for regulating skeletal muscle mass or function.

### Example 7

Determination of Absolute Force Measurement of a Muscle.

The extensor digitorum longus (EDL) and soleus muscles are removed, tendon-to-tendon from the casted mouse leg. A silk suture is tied to each tendon of the isolated muscles and the muscles are placed into a Plexiglas chamber filled with Ringer solution (137 mM sodium chloride, 24 mM sodium bicarbonate, 11 mM glucose, 5 mM potassium chloride, 1 mM magnesium sulfate, 1 mM sodium phosphate, 0.025 mM tubocurarine, all at pH 7.4 and oxygenated with 95% oxygen/5% carbon dioxide) constantly bubbled with 95% oxygen/5% carbon dioxide maintained at 25° C. Muscles are aligned horizontally between a servomotor lever arm (Model 305B-LR Cambridge Technology Inc., Watertown Mass., USA) and the stainless steel hook of a force transducer (Model BG-50; Kulite Semiconductor Products Inc., Leonia, N.J., USA) and field stimulated by pulses transmitted between two platinum electrodes placed longitudinally on either side of the muscle. Square wave pulses (0.2 ms duration) generated by a personal computer with a Labview board (Model PCI-MIO 16E-4), Labview Inc., Austin, Tex., USA) are amplified (Acurus power amplifier model A25, Dobbs Ferry, N.Y., USA) to increase titanic contraction. Stimulation voltage and muscle length (Lo) are adjusted to obtain maximum isometric twitch force. Maximum titanic force production (Po) is determined from the plateau of the frequency-force relationship.

### Example 8

Therapeutic Treatment of Skeletal Muscle Atrophy using a Human Antibody that is an Agonist of the hAR Receptor.

A human male subject weighing 50 kg and having significant muscular atrophy of the arms and legs due to prolonged bed rest, is treated to reverse the skeletal muscle atrophy. Once each week for a period of 3 months, 15 mls of an aqueous solution of pH 6 comprising an activating antibody of the hAR receptor is administered to the subject via intravenous injection. The solution comprises the following:

| Component | Concentration (mg/ml) |
|---|---|
| hAR receptor agonist antibody | 20 |
| L-histidine HCl | 0.47 |
| L-histidine | 0.3 |
| α, α-trehalose dihydrate | 20 |
| Polysorbate 20 | 0.1 |
| Bacteriostatic Sterile water | qs to 1 mL |

At the end of the treatment period, the subject exhibits measurable increases of muscle mass, strength and mobility of the arms and legs.

### Example 9

Prophylactic Treatment of Skeletal Muscle Atrophy using a Human Antibody that is an Agonist of the hAR Receptor.

A human female subject weighing 55 kg is scheduled for hip joint replacement surgery in one month. The subject is treated to enhance skeletal muscle mass prior to and following surgery to ultimately reduce the level of skeletal muscle atrophy due to muscle disuse during post-surgery recovery. Specifically, once each week for a period of 1 month prior to surgery and for 2 months post-surgery, 18 ml of an aqueous solution of pH 6.0 comprising an activating antibody of the hAR receptor, is administered to the subject via intravenous injection. The solution comprises the following:

| Component | Concentration (mg/ml) |
|---|---|
| hAR activating antibody | 20 |
| L-histidine HCl | 0.47 |
| L-histidine | 0.3 |
| α, α-trehalose dihydrate | 20 |
| Polysorbate 20 | 0.1 |
| Bacteriostatic Sterile water | qs to 1 mL |

At the end of the treatment period, the subject exhibits measurable preservation of muscle mass, strength and mobility of the arms and legs as compared to the subject's expected status without antibody therapy.

### Example 13

Prophylactic Treatment of Skeletal Muscle Atrophy using Amylin.

A human female subject weighing 60 kg is admitted to the hospital in a comatose state. The subject is treated by this method to prevent atrophy of the skeletal muscle of the entire body due to disuse in the comatose state. Specifically, once each day while in the coma, the subject is administered, via slow intravenous infusion, approximately 500 ml of an aqueous solution that is prepared by addition of 5 ml of the following stock solution to 500 ml of sterile saline:

| Component | Concentration (mg/ml) |
|---|---|
| Amylin | 12 |
| Sodium phosphate buffer, pH 7.4 | 140 |

As a result of treatment, the subject exhibits measurable preservation of skeletal muscle mass and function, and reduced physical therapy needs during the coma and after regaining consciousness, as compared to the subject's status without drug therapy.

Except as otherwise noted, all amounts including quantities, percentages, portions, and proportions, are understood to be modified by the word “about”, and amounts are not intended to indicate significant digits.

Except as otherwise noted, the articles “a”, “an”, and “the” mean “one or more”.

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed:

1. A method for identifying candidate compounds for regulating skeletal muscle mass or function, comprising:
a. contacting a test compound with an amylin receptor;
b. determining whether the test compound binds to the amylin receptor; and
c. identifying those test compounds that bind the amylin receptor as candidate compounds for regulating skeletal muscle mass or function.

2. A method for identifying candidate compounds for regulating skeletal muscle mass or function according to claim 1, further comprising:
a. selecting those compounds that bind amylin receptor and further determining whether the test compound increases muscle mass or function in a skeletal muscle atrophy model system; and
b. identifying those test compounds that modulate muscle mass or function as candidate compounds for regulating skeletal muscle mass or function.

3. The method for identifying candidate compounds according to claim 1 wherein, the amylin receptor comprises an amino acid sequence of calcitonin receptor that is 90% homologous to SEQ ID NOS: 2, 4, 6, 8, 10, 12, 14, 16, 18, 20, 22, 24, 26, 28, 30, 32, 34, 36, 38, 40, 42, or 44; and an amino acid sequence of either receptor activity modifying protein 1 (RAMP1) or receptor activity modifying protein 3 (RAMP3) that is 90% homologous to SEQ ID NOS: 46, 48, 50, 52, 54, 56, 58, 60, 62, 64, 66, 68, or 70.

4. The method for identifying candidate compounds according to claim 1 wherein, the amylin receptor comprises an amino acid sequence of calcitonin receptor of SEQ ID NOS: 2, 4, 6, 8, 10, 12, 14, 16, 18, 20, 22, 24, 26, 28, 30, 32, 34, 36, 38, 40, 42, or 44; and an amino acid sequence of either receptor activity modifying protein 1 (RAMP1) or receptor activity modifying protein 3 (RAMP3) of SEQ ID NOS: 46, 48, 50, 52, 54, 56, 58, 60, 62, 64, 66, 68, or 70.

5. A method for identifying candidate compounds for regulating skeletal muscle mass or function, comprising:
a. contacting a test compound with a cell expressing a functional amylin receptor;
b. determining whether the test compound activates the amylin receptor; and
c. identifying those test compounds which activate the amylin receptor as candidate compounds for regulating skeletal muscle mass or function.

6. A method for identifying candidate compounds for regulating skeletal muscle mass or function according to claim 5, further comprising:
c. selecting those compounds that bind amylin receptor and further determining whether the test compound increases muscle mass or function in a skeletal muscle atrophy model system; and
d. identifying those test compounds that modulate muscle mass or function as candidate compounds for regulating skeletal muscle mass or function.

7. The method for identifying candidate compounds according to claim 5 wherein, the amylin receptor comprises an amino acid sequence of calcitonin receptor that is 90% homologous to SEQ ID NOS: 2, 4, 6, 8, 10, 12, 14, 16, 18, 20, 22, 24, 26, 28, 30, 32, 34, 36, 38, 40, 42, or 44; and a sequence of either receptor activity modifying protein 1 (RAMP1) or receptor activity modifying protein 3 (RAMP3) that is 90% homologous to SEQ ID NOS: 46, 48, 50, 52, 54, 56, 58, 60, 62, 64, 66, 68, or 70.

8. The method for identifying candidate compounds according to claim 5 wherein, the amylin receptor comprises an amino acid sequence of calcitonin receptor of SEQ ID NOS: 2, 4, 6, 8, 10, 12, 14, 16, 18, 20, 22, 24, 26, 28, 30, 32, 34, 36, 38, 40, 42, or 44; and a sequence of either receptor activity modifying protein 1 (RAMP1) or receptor activity modifying protein 3 (RAMP3) of SEQ ID NOS: 46, 48, 50, 52, 54, 56, 58, 60, 62, 64, 66, 68, or 70.

9. The method for identifying candidate compounds according to claim 5 wherein, determining whether the test compound activates amylin receptor involves measuring cellular cAMP levels.

10. The method for identifying candidate compounds according to claim 5, wherein the method further comprises a reporter gene operatively associated with a cAMP responsive element and measuring the expression of the reporter gene is analogous to measuring cAMP levels.

11. A method for identifying candidate compounds for regulating skeletal muscle mass or function comprising:
a. contacting a test compound with a cell expressing a functional amylin receptor, and determining the level of activation of the amylin receptor;
b. contacting the test compound with a cell expressing a functional calcitonin receptor, and determining the level of activation of the calcitonin receptor;
c. comparing the level of amylin receptor activation in the cell expressing a functional amylin receptor to the level of calcitonin receptor activation in the cell expressing calcitonin receptor; and
d. identifying those test compounds that show selectivity for amylin receptor as candidate compounds for regulating skeletal muscle mass or function.

12. The method according to claim 11 wherein the candidate compound exhibits about 100-fold or greater selectivity for amylin receptor over calcitonin receptor.

13. The method according to claim 11 wherein the candidate compound exhibits about 1000-fold or greater selectivity for amylin receptor over calcitonin receptor.

14. A method for identifying candidate compounds for regulating skeletal muscle mass or function by identifying compounds that prolong or augment the agonist-induced activation of an amylin receptor or of an amylin receptor signal transduction pathway, comprising;
a. contacting a test compound with a first cell population which expresses a functional amylin receptor;
b. treating a second cell population with an amylin receptor agonist for a sufficient time and at a sufficient concentration to cause desensitization of the amylin receptor; further treating the second cell population with the test compound;
c. determining the level of activation of the amylin receptor in the first and second cell population; and
d. identifying those test compounds that prolong or augment the activation of an amylin receptor or of an amylin receptor signal transduction pathway as candidate compounds for regulating skeletal muscle mass or function.

15. A method for identifying candidate therapeutic compounds from a group of one or more candidate compounds which have been previously determined to bind, or activate an amylin receptor; or prolong, or augment the activation of an amylin receptor or of an amylin receptor signal transduction pathway comprising:
a. administering the candidate compound, or the candidate compound in conjunction with an amylin receptor agonist, to a non-human animal; and
b. determining whether the candidate compound regulates skeletal muscle mass or function in the treated animal.

16. A method for identifying candidate compounds for regulating skeletal muscle mass or function by identifying compounds that increase expression of amylin or amylin receptor, comprising:
a. contacting a test compound with a cell or cell lysate containing a reporter gene operatively associated with calcitonin receptor gene regulatory element, receptor activity modifying protein gene regulatory element, or an amylin gene regulatory element;
b. detecting expression of the reporter gene; and
c. identifying those test compounds that increase expression of the reporter gene as candidate compounds for regulating skeletal muscle mass or function.

17. A method for increasing skeletal muscle mass or function in a subject in which such an increase is desirable, comprising:
a. identifying a subject in which an increase in muscle mass or function is desirable; and
b. administering to the subject a safe and effective amount of a compound selected from the group consisting of an amylin receptor agonist, a compound that augments or prolongs amylin receptor or amylin receptor signal transduction pathway activation, an expression vector encoding a functional amylin receptor, an expression vector encoding a constitutively active amylin receptor, an expression vector encoding amylin and a compound that increases expression of an amylin receptor or amylin.

18. The method of claim 17 for increasing muscle mass or function wherein the subject in which such an increase is desirable is characterized by presence of muscle atrophy.

19. The method for treating skeletal muscle atrophy according to claim 17 wherein the compound is an amylin receptor agonist.

20. A pharmaceutical composition, comprising:
a. a safe and effective amount of an amylin receptor agonist; and
b. a pharmaceutically-acceptable carrier.

21. The pharmaceutical composition according to claim 20 wherein the amylin receptor agonist is a chimeric or human antibody specific for an amylin receptor.

22. The pharmaceutical composition according to claim 20 wherein the amylin receptor agonist is amylin.


SEQUENCE LISTING (70 sequences)

SEQ ID NO: 1 - DNA, 1530 bp, Homo sapiens
  CDS (73)..(1497)
ttgcttctattgagctgtgcccagccgcccagtgacagaattccaggacaaagagatcttcaaaaaccaaaaatgaggttcacatttacaagccggtgcttggcactgtttcttcttctaaatcacccaaccccaattcttcctgccttttcaaatcaaacctatccaacaatagagcccaagccatttctttacgtcgtaggacgaaagaagatgatggatgcacagtacaaatgctatgaccgaatgcagcagttacccgcataccaaggagaaggtccatattgcaatcgcacctgggatggatggctgtgctgggatgacacaccggctggagtattgtcctatcagttctgcccagattattttccggattttgatccatcagaaaaggttacaaaatactgtgatgaaaaaggtgtttggtttaaacatcctgaaaacaatcgaacctggtccaactatactatgtgcaatgctttcactcctgagaaactgaagaatgcatatgttctgtactatttggctattgtgggtcattctttgtcaattttcaccctagtgatttccctggggattttcgtgtttttcaggagccttggctgccaaagggtaaccctgcacaagaacatgtttcttacttacattctgaattctatgattatcatcatccacctggttgaagtagtacccaatggagagctcgtgcgaagggacccggtgagctgcaagattttgcattttttccaccagtacatgatggcctgcaactatttctggatgctctgtgaagggatctatcttcatacactcattgtcgtggctgtgtttactgagaagcaacgcttgcggtggtattatctcttgggctgggggttcccgctggtgccaaccactatccatgctattaccagggccgtgtacttcaatgacaactgctggctgagtgtggaaacccatttgctttacataatccatggacctgtcatggcggcacttgtggtcaatttcttctttttgctcaacattgtccgggtgcttgtgaccaaaatgagggaaacccatgaggcggaatcccacatgtacctgaaggctgtgaaggccaccatgatccttgtgcccctgctgggaatccagtttgtcgtctttccctggagaccttccaacaagatgcttgggaagatatatgattacgtgatgcactctctgattcatttccagggcttctttgttgcgaccatctactgcttctgcaacaatgaggtccaaaccaccgtgaagcgccaatgggcccaattcaaaattcagtggaaccagcgttgggggaggcgcccctccaaccgctctgctcgcgctgcagccgctgctgcggaggctggcgacatcccaatttacatctgccatcaggagccgaggaatgaaccagccaacaaccaaggcgaggagagtgctgagatcatccctttgaatatcatagagcaagagtcatctgcttgaatgtgaagcaaacacagtatcgtgatcactgag

SEQ ID NO: 2 - protein, 474 aa, Homo sapiens [sequence omitted]

SEQ ID NO: 3 - DNA, 3331 bp, Homo sapiens
  CDS (38)..(1462)
cagaattccaggacaaagagatcttcaaaaatcaaaaatgaggttcacatttacaagccggtgcttggcactgtttcttcttctaaatcacccaaccccaattcttcctgccttttcaaatcaaacctatccaacaatagagcccaagccatttctttacgtcgtaggacgaaagaagatgatggatgcacagtacaaatgctatgaccgaatgcagcagttacccgcataccaaggagaaggtccatattgcaatcgcacctgggatggatggctgtgctgggatgacacaccggctggagtattgtcctatcagttctgcccagattattttccggattttgatccatcagaaaaggttacaaaatactgtgatgaaaaaggtgtttggtttaaacatcctgaaaacaatcgaacctggtccaactatactatgtgcaatgctttcactcctgagaaactgaagaatgcatatgttctgtactatttggctattgtgggtcattctttgtcaattttcaccctagtgatttccctggggattttcgtgtttttcaggagccttggctgccaaagggtaaccctgcacaagaacatgtttcttacttacattctgaattctatgattatcatcatccacctggttgaagtagtacccaatggagagctcgtgcgaagggacccggtgagctgcaagattttgcattttttccaccagtacatgatggcctgcaactatttctggatgctctgtgaagggatctatcttcatacactcattgtcgtggctgtgtttactgagaagcaacgcttgcggtggtattatctcttgggctgggggttcccgctggtgccaaccactatccatgctattaccagggccgtgtacttcaatgacaactgctggctgagtgtggaaacccatttgctttacataatccatggacctgtcatggcggcacttgtggtcaatttcttctttttgctcaacattgtccgggtgcttgtgaccaaaatgagggaaacccatgaggcggaatcccacatgtacctgaaggctgtgaaggccaccatgatccttgtgcccctgctgggaatccagtttgtcgtctttccctggagaccttccaacaagatgcttgggaagatatatgattacgtgatgcactctctgattcatttccagggcttctttgttgcgaccatctactgcttctgcaacaatgaggtccaaaccaccgtgaagcgccaatgggcccaattcaaaattcagtggaaccagcgttgggggaggcgcccctccaaccgctctgctcgcgctgcagccgctgctgcggaggctggcgacatcccaatttacatctgccatcaggagctgaggaatgaaccagccaacaaccaaggcgaggagagtgctgagatcatccctttgaatatcatagagcaagagtcatctgcttgaatgtgaaggcaaacacagcatcgtgatcactgagccatcatttcctgggagaaagaccatgcatttaaagtattctccatcctcccaggaaccgaacatatcatttgtgaagaattattcagtgaatttgtccattgtaaatctgaagaaagttattcttggtactgttgctttgggagacagtctaggaatggagtctcccactgcaacttgtgaactccatcattcatccaggactgagatgcaaatgtcacagtaatgcaagcaaagtatcaaagaaaaacaatgaaattgacctagttcagatacagggtgctccttgtcaatactgagccatttatacctttgaaatattaaaatcactgtcaatatttttatttttaactctggattttgaattagattatttctgtatttggctatggatctgatttttaatttttttaaatttcagtcaattctgatgttactgagatgttttaccatccttacaatgtaaaccacatgaactacgtgacctctgcaagacaaagcggctttctaatagagagattagtaaatatgtgaagaaaaagacctgcatttggcaggaagatgtatgctttgaatgcaaaagaaatttagagtcaatttgctgaaaacattacatgctcagcttggttttggacaagcctgtccattgggcaggacctagctgttgtaaagaattggtcttaatgttgaatgtattttggttgctgatgtttataaactgagaggtcacaaagaatctatcactaaaaatttttacaaaactgccaaaaatataattcttagtggaagacaatactccctttaaagagagtttgccactcccctaaactccaggatttataaagcaaattactccaaggtttataaagcagattacctcttgcccttgggtgctatctagcagtaaaagataaatttgttgaatattggtaattaaaagactccacataagtccattaactgctttccacccagcttcaaagcttaaaaagagctcaggcttttccaggaagatccaggagggctaattagaaatcaacttgtggttgaccgcttgtttcttgttattaccaaacaggaggggaaaaaattaactgctccaaatttaaccataaatcaattcatgtttaacgtttctcattaaaatccagtattatattatcatatctctctttacttcccagtataagatttttgaaaatcctgaataaaccagtatcgttactggcacctgaaattaatttgtgaatttgcaacagtaatcagagttaccattatttaatttgtatgctaaatgaggaggtacattgaaaccctccaaatctccagtctcatctatgtcatattttgccactgcctttcagaagtgatttagttgtggaaagataataaattgatttgttatggttacatatttagcgcacccagagaaaattaattatatttctacagagaaaatgaatttgggatactaaagtagtttaagtctcctttactgaatgtaagggggggatcgaaaagaaggtatttttccaatcacagtgttatgtagtattgttctatttttgtttacaaacatggaaaacagagtatttctggcagctgtggtacaaatgtgataatatattgctaaaatattttagatgttattatgctaatatagtaggggttgaagaaaacaaaatagcttattatagaattgcacatagttctgcccaaattatgtgaaatgcttatgcttgtgtatatgtataaattaatacagagtacgttaaaagcaaaaagatgtatatttgcatatttttctaaagaaatatattattcatcttttcattc

SEQ ID NO: 4 - protein, 474 aa, Homo sapiens
MRFTFTSRCLALFLLLNHPTPILPAFSNQTYPTIEPKPFLYVVGRKKMMDAQYKCYDRMQQLPAYQGEGPYCNRTWDGWLCWDDTPAGVLSYQFCPDYFPDFDPSEKVTKYCDEKGVWFKHPENNRTWSNYTMCNAFTPEKLKNAYVLYYLAIVGHSLSIFTLVISLGIFVFFRSLGCQRVTLHKNMFLTYILNSMIIIIHLVEVVPNGELVRRDPVSCKILHFFHQYMMACNYFWMLCEGIYLHTLIVVAVFTEKQRLRWYYLLGWGFPLVPTTIHAITRAVYFNDNCWLSVETHLLYIIHGPVMAALVVNFFFLLNIVRVLVTKMRETHEAESHMYLKAVKATMILVPLLGIQFVVFPWRPSNKMLGKIYDYVMHSLIHFQGFFVATIYCFCNNEVQTTVKRQWAQFKIQWNQRWGRRPSNRSARAAAAAAEAGDIPIYICHQELRNEPANNQGEESAEIIPLNIIEQESSA

SEQ ID NO: 5 - DNA, 1425 bp, Homo sapiens
  CDS (1)..(1425)
atgaggttcacatttacaagccggtgcttggcactgtttcttcttctaaatcacccaaccccaattcttcctgccttttcaaatcaaacctatccaacaatagagcccaagccatttctttacgtcgtaggacgaaagaagatgatggatgcacagtacaaatgctatgaccgaatgcagcagttacccgcataccaaggagaaggtccatattgcaaccgcacctgggatggatggctgtgctgggatgacacaccggctggagtattgtcctatcagttctgcccagattattttccggattttgatccatcagaaaaggttacaaaatactgtgatgaaaaaggtgtttggtttaaacatcctgaaaacaatcgaacctggtccaactatactatgtgcaatgctttcactcctgagaaactgaagaatgcatatgttctgtactatttggctattgtgggtcattctttgtcaattttcaccctagtgatttccctggggattttcgtgtttttcaggagccttggctgccaaagggtaaccctgcacaagaacatgtttcttacttacattctgaattctatgattatcatcatccacctggttgaagtagtacccaatggagagctcgtgcgaagggacccggtgagctgcaagattttgcattttttccaccagtacatgatggcctgcaactatttctggatgctctgtgaagggatctatcttcatacactcattgtcgtggctgtgtttactgagaagcaacgcttgcggtggtattatctcttgggctgggggttcccgctggtgccaaccactatccatgctattaccagggccgtgtacttcaatgacaactgctggctgagtgtggaaacccatttgctttacataatccatggacctgtcatggcggcacttgtggtcaatttcttctttttgctcaacattgtccgggtgcttgtgaccaaaatgagggaaacccatgaggcggaatcccacatgtacctgaaggctgtgaaggccaccatgatccttgtgcccctgctgggaatccagtttgtcgtctttccctggagaccttccaacaagatgcttgggaagatatatgattacgtgatgcactctctgattcatttccagggcttctttgttgcgaccatctactgcttctgcaacaatgaggtccaaaccaccgtgaagcgccaatgggcccaattcaaaattcagtggaaccagcgttgggggaggcgcccctccaaccgctctgctcgcgctgcagccgctgctgcggaggctggcgacatcccaatttacatctgccatcaggagctgaggaatgaaccagccaacaaccaaggcgaggagagtgctgagatcatccctttgaatatcatagagcaagagtcatctgcttga

SEQ ID NO: 6 - protein, 474 aa, Homo sapiens; identical to SEQ ID NO: 4

SEQ ID NO: 7 - DNA, 1339 bp, Homo sapiens
  CDS (23)..(1306)
ccaaaaatgaggacgaaagaagatgatggatgcacagtacaaatgctatgaccgaatgcagcagttacccgcatatcaaggagaaggtccatattgcaatcgcacctgggatggatggctgtgctgggatgacacaccggctggagtattgtcctatcagttctgcccagattattttccggattttgatccatcagaaaaggttacaaaatactgtgatgaaaaaggtgtttggtttaaacatcctgaaaacaatcgaacctggtccaactatactatgtgcaatgctttcactcctgagaaactgaagaatgcatatgttctgtactatttggctattgtgggtcattctttgtcaattttcaccctagtgatttccctggggattttcgtgtttttcaggagccttggctgccaaagggtaaccctgcacaagaacatgtttcttacttacattctgaattctatgattatcatcatccacctggttgaagtagtacccaatggagagctcgtgcgaagggacccggtgagctgcaagattttgcattttttccaccagtacatgatggcctgcaactatttctggatgctctgtgaagggatctatcttcatacactcattgtcgtggctgtgtttactgagaagcaacgcttgcggtggtattatctcttgggctgggggttcccgctggtgccaaccactatccatgctattaccagggccgtgtacttcaatgacaactgctggctgagtgtggaaacccatttgctttacataatccatggacctgtcatggcggcacttgtggtcaatttcttctttttgctcaacattgtccgggtgcttgtgaccaaaatgagggaaacccatgaggcggaatcccacatgtacctgaaggctgtgaaggccaccatgacccttgtgcccctgctgggaatccagtttgtcgtctttccctggagaccttccaacaagatgcttgggaagatatatgattacgtgatgcactctctgattcatttccagggcttctttgttgcgaccatctactgcttctgcaacaatgaggtccaaaccaccgtgaagcgccaatgggcccaattcaaaattcagtggaaccagcgttgggggaggcgcccctccaaccgctctgctcgcgctgcagccgctgctgcggaggctggcgacatcccaatttacatctgccatcaggagctgaggaatgaaccagccaacaaccaaggcgaggagagtgctgagatcatccctttgaatatcatagagcaagagtcatctgcttgaatgtgaagcaaacacagtatcgtgatcactgag

SEQ ID NO: 8 - protein, 427 aa, Homo sapiens [sequence omitted]

SEQ ID NO: 9 - DNA, 3588 bp, Homo sapiens
  CDS (248)..(1720)
gtgcgcacgtccgcacctcaccctgcggctgacatctcctgcccaggagatgggcgctgaagcttgagcgcctgagtccctggagccacacctgcgaacaccctttgcttctattgagctgtgcccagccgcccagtgacagaattccagaataaatgattcccactgatccacccacttttgccaccccaggatgcaattttctggagagaagattagtggacaaagagatcttcaaaaatcaaaaatgaggttcacatttacaagccggtgcttggcactgtttcttcttctaaatcacccaaccccaattcttcctgccttttcaaatcaaacctatccaacaatagagcccaagccatttctttacgtcgtaggacgaaagaagatgatggatgcacagtacaaatgctatgaccgaatgcagcagttacccgcataccaaggagaaggtccatattgcaatcgcacctgggatggatggctgtgctgggatgacacaccggctggagtattgtcctatcagttctgcccagattattttccggattttgatccatcagaaaaggttacaaaatactgtgatgaaaaaggtgtttggtttaaacatcctgaaaacaatcgaacctggtccaactatactatgtgcaatgctttcactcctgagaaactgaagaatgcatatgttctgtactatttggctattgtgggtcattctttgtcaattttcaccctagtgatttccctggggattttcgtgtttttcagaaaattgacaactatttttcctttgaattggaaatataggaaggcattgagccttggctgccaaagggtaaccctgcacaagaacatgtttcttacttacattctgaattctatgattatcatcatccacctggttgaagtagtacccaatggagagctcgtgcgaagggacccggtgagctgcaagattttgcattttttccaccagtacatgatggcctgcaactatttctggatgctctgtgaagggatctatcttcatacactcattgtcgtggctgtgtttactgagaagcaacgcttgcggtggtattatctcttgggctgggggttcccgctggtgccaaccactatccatgctattaccagggccgtgtacttcaatgacaactgctggctgagtgtggaaacccatttgctttacataatccatggacctgtcatggcggcacttgtggtcaatttcttctttttgctcaacattgtccgggtgcttgtgaccaaaatgagggaaacccatgaggcggaatcccacatgtacctgaaggctgtgaaggccaccatgatccttgtgcccctgctgggaatccagtttgtcgtctttccctggagaccttccaacaagatgcttgggaagatatatgattacgtgatgcactctctgattcatttccagggcttctttgttgcgaccatctactgcttctgcaacaatgaggtccaaaccaccgtgaagcgccaatgggcccaattcaaaattcagtggaaccagcgttgggggaggcgcccctccaaccgctctgctcgcgctgcagccgctgctgcggaggctggcgacatcccaatttacatctgccatcaggagccgaggaatgaaccagccaacaaccaaggcgaggagagtgctgagatcatccctttgaatatcatagagcaagagtcatctgcttgaatgtgaagcaaacacagtatcgtgatcactgagccatcatttcctgggagaaagaccatgcatttaaagtattctccatcctcccaggaaccgaacatatcatttgtgaagaattattcagtgaatttgtccattgtaaatctgaagaaagttattcttggtactgttgctttgggagacagtctaggaatggagtctcccactgcaacttgtgaactccatcattcatccaggactgagatgcaaatgtcacagtaatgcaagcaaagtatcaaagaaaaacaatgaaattgacctagttcagatacagggtgctccttgtcaatactgagccatttatacctttgaaatattaaaatcactgtcaatatttttatttttaactctggattttgaattagattatttctgtatttggctatggatctgatttttaatttttttaaatttcagtcaattctgatgttactgagatgttttaccatccttacaatgtaaaccacatgaactacgtgacctctgcaagacaaagcggctttctaatagagagattagtaaatatgtgaagaaaaagacctgcatttggcaggaagatgtatgctttgaatgcaaaagaaatttagagtcaatttgctgaaaacattacatgctcagcttggttttggacaagcctgtccattgggcaggacctagctgttgtaaagaattggtcttaatgttgaatgtattttggttgctgatgtttataaactgagaggtcacaaagaatctatcactaaaaatttttacaaaactgccaaaaatataattcttagtggaagacaatactccctttaaagaaagagagtttgccactcccctaaactccaggatttataaagcaaattactccaaggtttataaagcagattacctcttgcccttgggtgctatctagcagtaaaagataaatttgttgaatattggtaattaaaagactccacataagtccattaactgctttccacccagcttcaaagcttaaaaagagctcaggcttttccaggaagatccaggagggctaattagaaatcaacttgtggttgaccgcttgtttcttgttattaccaaaacaggaggggaaaaaattaactgctccaaatttaaccataaatcaattcatgtttaacgtttctcattaaaatccagtattatattatcatatctctctttacttcccagtataagatttttgaaaatcctgaataaaccagtatcgttactggcacctgaaattaatttgtgaatttgcaacagtaatcagagttaccattatttaatttgtatgctaaatgaggaggtacattgaaaccctccaaatctccagtctcatctatgtcatattttgccactgcctttcagaagtgatttagttgtggaaagataataaattgatttgttatggttacatattcagcgcacccagagaaaattaattatatttctacagagaaaatgaatttgggatactaaagtagtttaagtctcctttactgaatgtaagggggggatcgaaaagaaggtatttttccaatcacagtgttatgtagtattgttctatttttgtttacaaacatggaaaacagagtatttctggcagctgtggtacaaatgtgataatatattgctaaaatattttagatgttattatgctaatatagtaggggttgaagaaaacaaaatagcttattatagaattgcacatagttctgcccaaattatgtgaaatgcttatgcttgtgtatatgtataaattaatacagagtacgttaaaagcaaaaagatgtatatttgcatatttttctaaagaaatatattattcatctttt

SEQ ID NO: 10 - protein, 490 aa, Homo sapiens [sequence omitted]

SEQ ID NO: 11 - DNA, 3588 bp, Homo sapiens
  CDS (248)..(1720)
gtgcgcacgtccgcacctcaccctgcggctgacatctcctgcccaggagatgggcgctgaagcttgagcgcctgagtccctggagccacacctgcgaacaccctttgcttctattgagctgtgcccagccgcccagtgacagaattccagaataaatgattcccactgatccacccacttttgccaccccaggatgcaattttctggagagaagattagtggacaaagagatcttcaaaaatcaaaaatgaggttcacatttacaagccggtgcttggcactgtttcttcttctaaatcacccaaccccaattcttcctgccttttcaaatcaaacctatccaacaatagagcccaagccatttctttacgtcgtaggacgaaagaagatgatggatgcacagtacaaatgctatgaccgaatgcagcagttacccgcataccaaggagaaggtccatattgcaatcgcacctgggatggatggctgtgctgggatgacacaccggctggagtattgtcctatcagttctgcccagattattttccggattttgatccatcagaaaaggttacaaaatactgtgatgaaaaaggtgtttggtttaaacatcctgaaaacaatcgaacctggtccaactatactatgtgcaatgctttcactcctgagaaactgaagaatgcatatgttctgtactatttggctattgtgggtcattctttgtcaattttcaccctagtgattttcctggggattttcgtgtttttcagaaaattgacaactatttttcctttgaattggaaatataggaaggcattgagccttggctgccaaagggtaaccctgcacaagaacatgtttcttacttacattctgaattctatgattatcatcatccacctggttgaagtagtacccaatggagagctcgtgcgaagggacccggtgagctgcaagattttgcattttttccaccagtacatgatggcctgcaactatttctggatgctctgtgaagggatctatcttcatacactcattgtcgtggctgtgtttactgagaagcaacgcttgcggtggtattatctcttgggctgggggttcccgctggtgccaaccactatccatgctattaccagggccgtgtacttcaatgacaactgctggctgagtgtggaaacccatttgctttacataatccatggacctgtcatggcggcacttgtggtcaatttcttctttttgctcaacattgtccgggtgcttgtgaccaaaatgagggaaacccatgaggcggaatcccacatgtacctgaaggctgtgaaggccaccatgatccttgtgcccctgctgggaatccagtttgtcgtctttccctggagaccttccaacaagatgcttgggaagatatatgattacgtgatgcactctctgattcatttccagggcttctttgttgcgaccatctactgcttctgcaacaatgaggtccaaaccaccgtgaagcgccaatgggcccaattcaaaattcagtggaaccagcgttgggggaggcgcccctccaaccgctctgctcgcgctgcagccgctgctgcggaggctggcgacatcccaatttacatctgccatcaggagccgaggaatgaaccagccaacaaccaaggcgaggagagtgctgagatcatccctttgaatatcatagagcaagagtcatctgcttgaatgtgaagcaaacacagtatcgtgatcactgagccatcatttcctgggagaaagaccatgcatttaaagtattctccatcctcccaggaaccgaacatatcatttgtgaagaattattcagtgaatttgtccattgtaaatctgaagaaagttattcttggtactgttgctttgggagacagtctaggaatggagtctcccactgcaacttgtgaactccatcattcatccaggactgagatgcaaatgtcacagtaatgcaagcaaagtatcaaagaaaaacaatgaaattgacctagttcagatacagggtgctccttgtcaatactgagccatttatacctttgaaatattaaaatcactgtcaatatttttatttttaactctggattttgaattagattatttctgtatttggctatggatctgatttttaatttttttaaatttcagtcaattctgatgttactgagatgttttaccatccttacaatgtaaaccacatgaactacgtgacctctgcaagacaaagcggctttctaatagagagattagtaaatatgtgaagaaaaagacctgcatttggcaggaagatgtatgctttgaatgcaaaagaaatttagagtcaatttgctgaaaacattacatgctcagcttggttttggacaagcctgtccattgggcaggacctagctgttgtaaagaattggtcttaatgttgaatgtattttggttgctgatgtttataaactgagaggtcacaaagaatctatcactaaaaatttttacaaaactgccaaaaatataattcttagtggaagacaatactccctttaaagaaagagagtttgccactcccctaaactccaggatttataaagcaaattactccaaggtttataaagcagattacctcttgcccttgggtgctatctagcagtaaaagataaatttgttgaatattggtaattaaaagactccacataagtccattaactgctttccacccagcttcaaagcttaaaaagagctcaggcttttccaggaagatccaggacggctaattagaaatcaacttgtggttgaccgcttgtttcttgttattaccaaaacaggaggggaaaaaattaactgctccaaatttaaccataaatcaattcatgtttaacgtttctcattaaaatccagtattatattatcatatctctctttacttcccagtataagatttttgaaaatcctgaataaaccagtatcgttactggcacctgaaattaatttgtgaatttgcaacagtaatcagagttaccattatttaatttgtatgctaaatgaggaggtacattgaaaccctccaaatctccagtctcatctatgtcatattttgccactgcctttcagaagtgatttagttgtggaaagataataaattgatttgttatggttacatattcagcgcacgcagagaaaattaattatatttctacagagaaaatgaatttgggatactaaagtagtttaagtctcctttactgaatgtaagggggggatcgaaaagaaggtatttttccaatcacagtgttatgtagtattgttctatttttgtttacaaacatggaaaacagagtatttctggcagctctcgtacaaatgtgataatatattgctaaaatattttagatgttattatgctaatatagtaggggttgaagaaaacaaaatagcttattatagaattgcacatagttctgcccaaattatgtgaaatgcttatgcttgtgtatatgtataaattaatacacactacgttaaaagcaaaaagatgtatatttgcatatttttctaaagaaatatattattcatctttt

SEQ ID NO: 12 - protein, 490 aa, Homo sapiens
MRFTFTSRCLALFLLLNHPTPILPAFSNQTYPTIEPKPFLYVVGRKKMMDAQYKCYDRMQQLPAYQGEGPYCNRTWDGWLCWDDTPAGVLSYQFCPDYFPDFDPSEKVTKYCDEKGVWFKHPENNRTWSNYTMCNAFTPEKLKNAYVLYYLAIVGHSLSIFTLVIFLGIFVFFRKLTTIFPLNWKYRKALSLGCQRVTLHKNMFLTYILNSMIIIIHLVEVVPNGELVRRDPVSCKILHFFHQYMMACNYFWMLCEGIYLHTLIVVAVFTEKQRLRWYYLLGWGFPLVPTTIHAITRAVYFNDNCWLSVETHLLYIIHGPVMAALVVNFFFLLNIVRVLVTKMRETHEAESHMYLKAVKATMILVPLLGIQFVVFPWRPSNKMLGKIYDYVMHSLIHFQGFFVATIYCFCNNEVQTTVKRQWAQFKIQWNQRWGRRPSNRSARAAAAAAEAGDIPIYICHQEPRNEPANNQGEESAEIIPLNIIEQESSA

SEQ ID NO: 13 - DNA, 3416 bp, Homo sapiens
  CDS (52)..(594)
gaattcgcggccgccagaattccaggacaaagagatcttcaaaaatcaaaaatgaggttcacatttacaagccggtgcttggcactgtttcttcttctaaatcacccaaccccaattcttcctgccttttcaaatcaaacctatccaacaatagagcccaagccatttctttacgtcgtaggacgaaagaagatgatggatgcacagtacaaatgctatgaccgaatgcagcagttacccgcataccaaggagaaggtccatattgcaatcgcacctgggatggatggctgtgctgggatgacacaccggctggagtattgtcctatcagttctgcccagattattttccggattttgatccatcagaaaaggttacaaaatactgtgatgaaaaaggtgtttggtttaaacatcctgaaaacaatcgaacctggtccaactatactatgtgcaatgctttcactcctgagaaactgaagaatgcatatgttctgtactatttggctattgtgggtcattctttgtcaattttcaccctagtgatttccctggggattttcgtgtttttcagttctcatgtttatcatgaataacacgtgatcctaggagccttggctgccaaagggtaaccctgcacaagaacatgtttcttacttacattctgaattctatgattatcatcatccacctggttgaagtagtacccaatggagagctcgtgcgaagggacccggtgagctgcaagattttgcattttttccaccagtacatgatggcctgcaactatttctggatgctctgtgaagggatctatcttcatacactcattgtcgtggctgtgtttactgagaagcaacgcttgcggtggtattatctcttgggctgggggttcccgctggtgccaaccactatccatgctattaccagggccgtgtacttcaatgacaactgctggctgagtgtggaaacccatttgctttacataatccatggacctgtcatggcggcacttgtggtcaatttcttctttttgctcaacattgtccgggtgcttgtgaccaaaatgagggaaacccatgaggcggaatcccacatgtacctgaaggctgtgaaggccaccatgatccttgtgcccctgctgggaatccagtttgtcgtctttccctggagaccttccaacaagatgcttgggaagatatatgattacgtgatgcactctctgattcatttccagggcttctttgttgcgaccatctactgcttctgcaacaatgaggtccaaaccaccgtgaagcgccaatgggcccaattcaaaattcagtggaaccagcgttgggggaggcgcccctccaaccgctctgctcgcgctgcagccgctgctgcggaggctggcgacatcccaatttacatctgccatcaggagctgaggaatgaaccagccaacaaccaaggcgaggagagtgctgagatcatccctttgaatatcatagagcaagagtcatctgcttgaatgtgaaggcaaacacagcatcgtgatcactgagccatcatttcctgggagaaagaccatgcatttaaagtattctccatcctcccaggaaccgaacatatcatttgtgaagaattattcagtgaatttgtccattgtaaatctgaagaaagttattcttggtactgttgctttgggagacagtctaggaatggagtctcccactgcaacttgtgaactccatcattcatccaggactgagatgcaaatgtcacagtaatgcaagcaaagtatcaaagaaaaacaatgaaattgacctagttcagatacagggtgctccttgtcaatactgagccatttatacctttgaaatattaaaatcactgtcaatatttttatttttaactctggattttgaattagattatttctgtatttggctatggatctgatttttaatttttttaaatttcagtcaattctgatgttactgagatgttttaccatccttacaatgtaaaccacatgaactacgtgacctctgcaagacaaagcggctttctaatagagagattagtaaatatgtgaagaaaaagacctgcatttggcaggaagatgtatgctttgaatgcaaaagaaatttagagtcaatttgctgaaaacattacatgctcagcttggttttggacaagcctgtccattgggcaggacctagctgttgtaaagaattggtcttaatgttgaatgtattttggttgctgatgtttataaactgagaggtcacaaagaatctatcactaaaaatttttacaaaactgccaaaaatataattcttagtggaagacaatactccctttaaagagagtttgccactcccctaaactccaggatttataaagcaaattactccaaggtttataaagcagattacctcttgcccttgggtgctatctagcagtaaaagataaatttgttgaatattggtaattaaaagactccacataagtccattaactgctttccacccagcttcaaagcttaaaaagagctcaggcttttccaggaagatccaggagggctaattagaaatcaacttgtggttgaccgcttgtttcttgttattaccaaacaggaggggaaaaaattaactgctccaaatttaaccataaatcaattcatgtttaacgtttctcattaaaatccagtattatattatcatatctctctttacttcccagtataagatttttgaaaatcctgaataaaccagtatcgttactggcacctgaaattaatttgtgaatttgcaacagtaatcagagttaccattatttaatttgtatgctaaatgaggaggtacattgaaaccctccaaatctccagtctcatctatgtcatattttgccactgcctttcagaagtgatttagttgtggaaagataataaattgatttgttatggttacatatttagcgcacccagagaaaattaattatatttctacagagaaaatgaatttgggatactaaagtagtttaagtctcctttactgaatgtaagggggggatcgaaaagaaggtatttttccaatcacagtgttatgtagtattgttctatttttgtttacaaacatggaaaacagagtatttctggcagctgtggtacaaatgtgataatatattgctaaaatattttagatgttattatgctaatatagtaggggttgaagaaaacaaaatagcttattatagaattgcacatagttctgcccaaattatgtgaaatgcttatgcttgtgtatatgtataaattaatacagagtacgttaaaagcaaaaagatgtatatttgcatatttttctaaagaaatatattattcatcttttcattcaaaaaaaaaaaaaaagagctcaattcccggggatcc

SEQ ID NO: 14 - protein, 180 aa, Homo sapiens
MRFTFTSRCLALFLLLNHPTPILPAFSNQTYPTIEPKPFLYVVGRKKMMDAQYKCYDRMQQLPAYQGEGPYCNRTWDGWLCWDDTPAGVLSYQFCPDYFPDFDPSEKVTKYCDEKGVWFKHPENNRTWSNYTMCNAFTPEKLKNAYVLYYLAIVGHSLSIFTLVISLGIFVFFSSHVYHE

SEQ ID NO: 15 - DNA, 1493 bp, Homo sapiens
  CDS (11)..(1483)
gaattccaccatgaggttcacatttacctcgaggtgcttagcgctgtttcttcttctaaatcacccaaccccaattctacctgccttttcgaatcaaacctatccaacgatcgagcccaagccatttctttacgtcgtcggccgaaagaagatgatggatgcgcagtacaaatgctatgaccgaatgcagcaattgcccgcataccaaggagaaggtccatattgcaatcgtacgtgggatggatggctgtgctgggatgacacgccggctggagtcttaagctatcagttctgcccagattattttccggattttgatccatcagaaaaggttacaaagtactgtgatgaaaaaggtgtttggtttaaacatcctgaaaacaatcgcacgtggtccaactatactatgtgcaatgctttcactcctgagaaactgaaaaatgcatacgttctgtactatttggctattgtgggtcattctttatcgattttcaccctagtgatatccctcgggattttcgtgtttttcagaaagcttacaactatttttccattgaattggaaatataggaaggccttgtccttaggctgccaaagggtaaccctgcacaagaacatgtttcttacgtacattctaaattctatgatcatcatcatccacctggtggaggtagtacccaatggagagctcgtgcgaagggacccggtcagctgcaagattttgcattttttccaccagtacatgatggcatgcaactatttctggatgctctgtgaagggatctatcttcatacactcattgtcgtggctgtgtttactgagaagcaacgtctacggtggtattatctcttgggctgggggttcccgctggtaccaaccactatccatgctattacgcgcgccgtgtacttcaatgacaactgctggctgagtgtggaaacccatttgctttacataatccatggaccggtcatggcggcactagtggtgaatttcttctttttgctcaacattgtacgcgtgcttgtgaccaaaatgagggaaactcatgaggcggaatcccacatgtacctgaaggccgtgaaggccaccatgatccttgtgcccctgctggggatccagtttgtcgtctttccctggcgcccttccaacaagatgcttgggaagatctatgattacgtgatgcactcattaattcatttccagggcttctttgtggccaccatctactgcttctgcaacaatgaggtccaaaccaccgtgaagcgccaatgggcccaattcaaaattcagtggaaccagcgttggggaagacgcccctccaaccgcagtgcacgcgctgcagccgctgctgcggaggctggcgacatcccaatttacatatgccatcaggagcctaggaatgaaccagccaacaaccaaggcgaggagtccgcggagatcatccctttgaatattatagagcaagagtcatctgcttgatagcggccgc

SEQ ID NO: 16 - protein, 490 aa, Homo sapiens [sequence omitted]

SEQ ID NO: 17 - DNA, 1472 bp, Cavia porcellus
  CDS (20)..(1456)
cgcccgggcaggtccgaaaatgaggttcacttttacacgccagttcttggcattcttcatcctcatcagtaacccagcttcaattcttcccaggtctgagaatctcacctttccgacatttgagcctgagccatatctgtattctgtcggacgcaagaagttggtggacgctcagtacaggtgctatgaccgtatgcaacagttacctccttatgaaggagaaggcccctactgcaatcggacgtgggacgggtggatgtgctgggacgatactccagccggagtactctccgttcagttatgcccggattactttccagactttgatccaacagaaaaggttacaaagtactgcgatgaaagtggggtttggtttaagcatcctgagaacaatcgaacctggtccaactataccttgtgcaacgcctttactcctgagaagctgcagaatgcttatgttctgtactatctggctatcgtgggtcactctatgtcgattatcaccttggtggtttctctgggaatttttgtgtatttcaggagtcttggctgccagcgggtgaccctgcacaagaacatgttcctgacgtacattctgaactccatgatcatcatcatccacctggttgaggtcgtgcccaatggagagctggtgcgcaaggacccggtgagctgcaagattttgcacttctttcaccagtacatgatggcctgcaattatttctggatgctctgtgaagggatctatcttcatacgctcattgtggtgtctgtgtttaacgaggcaaagcatctgcgctggtattatctcctgggctgggggttcccactggtgccaaccactatccatgctattactcgggcactgtacttcaatgacaactgctggataagtgtggatacccacttgctgtacatcatccatggccctgtcatggtggcgctggtggtcaatttcttctttttgctcaacatcgtccgagtgctggtgactaagatgagggaaacccatgaggccgagtcctacatgtacctgaaggccgtgaaggccaccatgatcctggtgcccctgctgggaatccagtttgtcgtctttccctggaggccttccaacaaagtgcttgggaagatctatgactacttcatgcactctctgattcacttccagggattcttcgttgcgactatctactgcttctgcaacaacgaggtccaaactaccctgaagcgccagtgggcccagttcaagatccaatggaaccagcgctggggaacccgcccctccaaccgctcggctgctgctcgagctgcagctgctgctgctgaggctggcggtgacaatatcccagtttacatctgccaccaggagccgaggaatgatccacccaacaaccaaggcgaggagggtgctgagatgatcgttttgaacatcatcgagaaagagtcatctgcttgaagtgtaaatacacaca

SEQ ID NO: 18 - protein, 478 aa, Cavia porcellus
MRFTFTRQFLAFFILISNPASILPRSENLTFPTFEPEPYLYSVGRKKLVDAQYRCYDRMQQLPPYEGEGPYCNRTWDGWMCWDDTPAGVLSVQLCPDYFPDFDPTEKVTKYCDESGVWFKHPENNRTWSNYTLCNAFTPEKLQNAYVLYYLAIVGHSMSIITLVVSLGIFVYFRSLGCQRVTLHKNMFLTYILNSMIIIIHLVEVVPNGELVRKDPVSCKILHFFHQYMMACNYFWMLCEGIYLHTLIVVSVFNEAKHLRWYYLLGWGFPLVPTTIHAITRALYFNDNCWISVDTHLLYIIHGPVMVALVVNFFFLLNIVRVLVTKMRETHEAESYMYLKAVKATMILVPLLGIQFVVFPWRPSNKVLGKIYDYFMHSLIHFQGFFVATIYCFCNNEVQTTLKRQWAQFKIQWNQRWGTRPSNRSAAARAAAAAAEAGGDNIPVYICHQEPRNDPPNNQGEEGAEMIVLNIIEKESSA

SEQ ID NO: 19 - DNA, 3555 bp, Oryctolagus cuniculus
  CDS (276)..(1700)
tcagcccctcgctcgctggcttgcggacctccgtggggagcagagaagcgcgcagagtgcctcgcctcgccctgggctgccgccgcctgcccaggagatgggcgcccgtgcctgacttgctaccaccacctgagaacgcccttcgcgttccctgggttgtgcccgccgcgactgagagaattcaggaacccaacactttgattaaggagaagactgaggcagagatggcgcatctccctcccagcaggatgaagagagatcttcagaaaccaaaaatgaagttcactcttacgtggcggtgctttgcgctgttcctccttctacatcaaccaacaccagttaaccctgcctcttcaaatgacacccatccaacagttgaacctgagccatttctgtacgtcataggacgcaagaagctgatggatgcacagtacaaatgctatgaccgaatggaacagctacctccataccaaggagaaggtccctactgcaaccggacctgggatggatggatgtgctgggacgacactccagctggagtcctgggcttccagtactgcccagattattttccagacttcgatccaacagaaaaggttacaaaatattgtgatgaaacaggagtttggtttaaacaccctgggaacaaccaaacctggtccaactatactatgtgtaatgcgttcactcctgagaaactacagaatgcgtacgtgctatactatctggctattgtgggtcattctctgtcgattttcaccttggtgatttccttggggatttttaagtgtttcaggagtcttggttgccagcgggtgaccctgcataagaacatgtttctgacttacattctgaactctatgattatcatcatccacctggttgaagttgtgcccaacggcgagctggtgagaagggatccggtgagctgcaaggttctgcattttttccaccagtacatgatgtcctgcaactatttctggatgctctgcgaagggatctacctgcacacgctgatcgtggtggccgtgtttgccaagcagcagcacctgcgctggtactacctcctgggctgggggttcccactggtgccaaccactatccacgctattactcgagcaatctacttcaatgacaactgctggatgagcgtggagacccatctgctttacatcatccatggcccagtgatggcagctctggtggtcaatttcttctttttgctcaacatcgtccgggtgctagtgaccaagatgagggaaaccctcgaagcagagtcacacatgtacctgaaggctgtgaaggccaccatgatcctggtgcccctgctgggaatccagtttgtcgtcttcccctggaggccctccaacaagattcttgggaagatctacgattacctcatgcactccctgattcacttccagggattctttgtggcgactatctactgtttctgcaacaatgaggtccagaccaccgtgaagcgccaatgggtacagtttaaaatccaatggaaccagcgctggggaagacgccccgcccaccgttccgtttcccgcactgcagcctcggctgaggaaggcggcatccccgtttatatctaccaccaggagcccaggaacgacccggcccacagcctaggcgaggagggtgccgagatcatccccttgaacatcatcgagcaagagtcatctgcttgactgtgaagccaacacagcatcgtgctcactgagccttcatcacctgggggaaagatagaccatgcatttaaagtgacccccctcctccaggagctgagcatcatatttgtgaagaattagtaagtgaatttgtcatagtgaatctggagagagttatcttgtactattgctctgggagacagtctaggaatggagtctcccactgcaacttgtgaactccatcattcacccaagactgaaatgcagatgacatagtacaacgaaggtatccaagaaaaacacaaattgacctagtgcagatacagggtgctccttgttaatcttgagccatttatcctttgaaaaattaaaatcactgtcaatattttttctttttaactctagattttgaatcagactttctgtatttggctatggatctgatttttaatctttttatttcgatcaattctgatgtattgaaatgttctaccatccatcgtgtaaaccgcacaaattacacgacctctgtggacacagtggctttttaacatcaagatgactaagcacgctggggaagaggggaagagacctgcatctggcaggaagacctaatctttgaaagacaaatttagattcaatttcgtggtaatagtacgtgttcagcttggctttggataagcctgtccattgggcaggcccagagtgctgtaaggaattggtctaatgttccgataatgcgtttgtttgctgacattataaacggaggtcacaaagaatccatcacaaaatttttcagaaaactgccaaaatcataattcttcatggaacaaaatgctcttaaaagagttttccactttcctaaactccaggatttataaagcaaatcactccaaggtttataaagcagattacctcttgcccttgggtgctatctagcagtaaaagacaaatttgtttaagactggtaattacaagactccatataagtccattaactgccttccaccctgcttcaaagcttaacaagatctggcgttcccaggaagattcagtagtgctaattggaaatcagttgtggttgacctcttgtttgctgctattagcaaaacaggagggccaaaatgtaactcctacaagtttaaccatattaattcatgttcaaagtaaaatttctcataaaaacccagtagtttatttccatcatctctttagttctcgagcataagatcctttgaaaatccctggataacctggaattatcactggcatctgaatttaatttgtgaatttgcaacagtaatagttctcatttaatttggatgctaaataagttgaaactttccaaatctccagtctcatctatatcatttatgccactgcctttcagaagcaattaagttgtggaaaaacaagcaattgatttgctctggttacacatttagtgcactcagagaaacattctgtttctccagtgaaaatgtattttggatactaaagtagtttaagtctcctttactgcacgtaagggaggaattgaaaagaaggtattttttcaatcacagtgttatgtattaacattcctatttttgtttacagacatgaaaaacagtatttcaggcagctctagtacaaatgtgataatatattgctaaaatattttaggtgttattgtgctaatatagtaggggctgaagaacacaaaatagcttaaaatagaattcgacatagtgccaaaatgatgtgaaatgcttatgttatatatatatgtgtatgtgtgtataaattaatagagtatgtgaaaagcaaaaagatgtatatttgcattatttttctacagaaatatattgttcatctcttcattcattaaaaaaaa

SEQ ID NO: 20 - protein, 474 aa, Oryctolagus cuniculus
MKFTLTWRCFALFLLLHQPTPVNPASSNDTHPTVEPEPFLYVIGRKKLMDAQYKCYDRMEQLPPYQGEGPYCNRTWDGWMCWDDTPAGVLGFQYCPDYFPDFDPTEKVTKYCDETGVWFKHPGNNQTWSNYTMCNAFTPEKLQNAYVLYYLAIVGHSLSIFTLVISLGIFKCFRSLGCQRVTLHKNMFLTYILNSMIIIIHLVEVVPNGELVRRDPVSCKVLHFFHQYMMSCNYFWMLCEGIYLHTLIVVAVFAKQQHLRWYYLLGWGFPLVPTTIHAITRAIYFNDNCWMSVETHLLYIIHGPVMAALVVNFFFLLNIVRVLVTKMRETLEAESHMYLKAVKATMILVPLLGIQFVVFPWRPSNKILGKIYDYLMHSLIHFQGFFVATIYCFCNNEVQTTVKRQWVQFKIQWNQRWGRRPAHRSVSRTAASAEEGGIPVYIYHQEPRNDPAHSLGEEGAEIIPLNIIEQESSA

SEQ ID NO: 21 - DNA, 3513 bp, Oryctolagus cuniculus
  CDS (276)..(1658)
tcagcccctcgctcgctggcttgcggacctccgtggggagcagagaagcgcgcagagtgcctcgcctcgccctgggctgccgccgcctgcccaggagatgggcgcccgtgcctgacttgctaccaccacctgagaacgcccttcgcgttccctgggttgtgcccgccgcgactgagagaattcaggaacccaacactttgattaaggagaagactgaggcagagatggcgcatctccctcccagcaggatgaagagagatcttcagaaaccaaaaatgaagttcactcttacgtggcggtgctttgcgctgttcctccttctacatcaaccaacaccagttaaccctgcctcttcaaatgacacccatccaacagttgaacctgagccatttctgtacgtcataggacgcaagaagctgatggatgcacagtacaaatgctatgaccgaatggaacagctacctccataccaaggagaaggtccctactgcaaccggacctgggatggatggatgtgctgggacgacactccagctggagtcctgggcttccagtactgcccagattattttccagacttcgatccaacagaaaaggttacaaaatattgtgatgaaacaggagtttggtttaaacaccctgggaacaaccaaacctggtccaactatactatgtgtaatgcgttcactcctgagaaactacagaatgcgtacgtgctatactatctggctattgtgggtcattctctgtcgattttcaccttggtgatttccttggggatttttaagtgtttcaggagtcttggttgccagcgggtgaccctgcataagaacatgtttctgacttacattctgaactctatgattatcatcatccacctggttgaagttgtgcccaacggcgagctggtgagaagggatccggtgagctgcaaggttctgcattttttccaccagtacatgatgtcctgcaactatttctggatgctctgcgaagggatctacctgcacacgctgatcgtggtggccgtgtttgccaagcagcagcacctgcgctggtactacctcctgggctgggggttcccactggtgccaaccactatccacgctattactcgagcaatctacttcaatgacaactgctggatgagcgtggagacccatctgctttacatcatccatggcccagtgatggcagctctggtggtcaatttcttctttttgctcaacatcgtccgggtgctagtgaccaagatgagggaaaccctcgaagcagagtcacacatgtacctgaaggctgtgaaggccaccatgatcctggtgcccctgctgggaatccagtttgtcgtcttcccctggaggccctccaacaagattcttgggaagatctacgattacctcatgcactccctgattcacttccaggtccagaccaccgtgaagcgccaatgggtacagtttaaaatccaatggaaccagcgctggggaagacgccccgcccaccgttccgtttcccgcactgcagcctcggctgaggaaggcggcatccccgtttatatctaccaccaggagcccaggaacgacccggcccacagcctaggcgaggagggtgccgagatcatccccttgaacatcatcgagcaagagtcatctgcttgactgtgaagccaacacagcatcgtgctcactgagccttcatcacctgggggaaagatagaccatgcatttaaagtgacccccctcctccaggagctgagcatcatatttgtgaagaattagtaagtgaatttgtcatagtgaatctggagagagttatcttgtactattgctctgggagacagtctaggaatggagtctcccactgcaacttgtgaactccatcattcacccaagactgaaatgcagatgacatagtacaacgaaggtatccaagaaaaacacaaattgacctagtgcagatacagggtgctccttgttaatcttgagccatttatcctttgaaaaattaaaatcactgtcaatattttttctttttaactctagattttgaatcagactttctgtatttggctatggatctgatttttaatctttttatttcgatcaattctgatgtattgaaatgttctaccatccatcgtgtaaaccgcacaaattacacgacctctgtggacacagtggctttttaacatcaagatgactaagcacgctggggaagaggggaagagacctgcatctggcaggaagacctaatctttgaaagacaaatttagattcaatttcgtggtaatagtacgtgttcagcttggctttggataagcctgtccattgggcaggcccagagtgctgtaaggaattggtctaatgttccgataatgcgtttgtttgctgacattataaacggaggtcacaaagaatccatcacaaaatttttcagaaaactgccaaaatcataattcttcatggaacaaaatgctcttaaaagagttttccactttcctaaactccaggatttataaagcaaatcactccaaggtttataaagcagattacctcttgcccttgggtgctatctagcagtaaaagacaaatttgtttaagactggtaattacaagactccatataagtccattaactgccttccaccctgcttcaaagcttaacaagatctggcgttcccaggaagattcagtagtgctaattggaaatcagttgtggttgacctcttgtttgctgctattagcaaaacaggagggccaaaatgtaactcctacaagtttaaccatattaattcatgttcaaagtaaaatttctcataaaaacccagtagtttatttccatcatctctttagttctcgagcataagatcctttgaaaatccctggataacctggaattatcactggcatctgaatttaatttgtgaatttgcaacagtaatagttctcatttaatttggatgctaaataagttgaaactttccaaatctccagtctcatctatatcatttatgccactgcctttcagaagcaattaagttgtggaaaaacaagcaattgatttgctctggttacacatttagtgcactcagagaaacattctgtttctccagtgaaaatgtattttggatactaaagtagtttaagtctcctttactgcacgtaagggaggaattgaaaagaaggtattttttcaatcacagtgttatgtattaacattcctatttttgtttacagacatgaaaaacagtatttcaggcagctctagtacaaatgtgataatatattgctaaaatattttaggtgttattgtgctaatatagtaggggctgaagaacacaaaatagcttaaaatagaattcgacatagtgccaaaatgatgtgaaatgcttatgttatatatatatgtgtatgtgtgtataaattaatagagtatgtgaaaagcaaaaagatgtatatttgcattatttttctacagaaatatattgttcatctcttcattcattaaaaaaaa

SEQ ID NO: 22 - protein, 460 aa, Oryctolagus cuniculus
MKFTLTWRCFALFLLLHQPTPVNPASSNDTHPTVEPEPFLYVIGRKKLMDAQYKCYDRMEQLPPYQGEGPYCNRTWDGWMCWDDTPAGVLGFQYCPDYFPDFDPTEKVTKYCDETGVWFKHPGNNQTWSNYTMCNAFTPEKLQNAYVLYYLAIVGHSLSIFTLVISLGIFKCFRSLGCQRVTLHKNMFLTYILNSMIIIIHLVEVVPNGELVRRDPVSCKVLHFFHQYMMSCNYFWMLCEGIYLHTLIVVAVFAKQQHLRWYYLLGWGFPLVPTTIHAITRAIYFNDNCWMSVETHLLYIIHGPVMAALVVNFFFLLNIVRVLVTKMRETLEAESHMYLKAVKATMILVPLLGIQFVVFPWRPSNKILGKIYDYLMHSLIHFQVQTTVKRQWVQFKIQWNQRWGRRPAHRSVSRTAASAEEGGIPVYIYHQEPRNDPAHSLGEEGAEIIPLNIIEQESSA

SEQ ID NO: 23 - DNA, 2144 bp, Sus scrofa
  CDS (256)..(1704)
ggaaggacggccagccccccagaccctgggagggctgcgccgcgcctttctcggaccttcctgggctggagaggtgcgcgccgtccgcacctcgcccaggggctggaatctccaatccaggagatgcgcgctggagcctgagttcctgagtctctggagccaccacctgcgaatgaccttcgcttccactgagctgtgcccggcagcccagtgagactgctccaggactgacaagatcatcttcaaaaatcaaaaatgaggttcacacttacacgctggtgcttgaccctcttcatctttctgaatcggccgttaccagttcttcctgacagtgcagatggcgcccacactccaacacttgagccggagccatttctctacattttagggaaacagcgaatgttagaagcacagcaccgatgctatgaccgaatgcaaaagttacccccataccaaggagaaggtctgtactgcaaccgtacctgggatggatggtcctgctgggatgacacaccggctggagtgctcgccgagcagtactgcccagactactttcctgactttgatgcagcagaaaaagttaccaagtactgtggtgaagacggggattggtatcgacaccctgagagcaacatatcctggtccaactacactatgtgcaatgctttcactcctgacaaactgcagaatgcgtacattctgtactacttggctattgtgggtcattctttgtcgattttgaccttgctgatttccctggggattttcatgtttctcaggagcattagctgccaacgggtgactctgcacaagaacatgtttctgacttacgttctgaattctattatcatcatcgtccacctggttgtgatcgtgcccaacggagagctggtgaaaagggatccgccgatctgcaaggttctgcacttcttccaccagtacatgatgtcatgcaattatttctggatgctctgcgagggggtctatcttcacactctgatcgtggtgtccgtgttcgctgagggacagcgcctgtggtggtatcatgtcctgggctgggggttcccgctgattccaactactgctcatgccattactagggcagtgttgttcaatgacaactgctggctgagtgtggacaccaacctgctttacatcatccacggccctgtcatggcagcactggtggtcaacttcttctttttgctcaacatcctccgcgtgctcgtgaagaaactgaaggaaagccaggaggcggaatcccacatgtacctgaaggcagtgcgggccactctgatcttggtgcccctgctgggcgtccagtttgtcgtccttccctggaggccttccaccccactgcttgggaagatctacgactacgtggtgcactctctgatccacttccagggattcttcgttgcgattatctactgcttctgcaaccacgaggttcagggagccctgaagcgccagtggaaccaataccaggcccagcgctgggctggccgccgctccacccgggccgccaacgctgcggccgccactgccgctgccgccgccgccctggccgagactgtggagatccctgtctacatctgccaccaggagccgagggaggaacccgccggcgaagagccggtcgtggaggtggagggtgttgaggtcatcgccatggaagtcctcgagcaagagacatctgcctgaacgtgacgcaaatacagcatcgtgatcactgagtcctcacttcccgggagaaagaccaaccttgcatccaaaatgatttccatcctccccgggggcaacatctcatttgtgagaattattcagtgaatttttccatcgtgaacctgaagtaagttttacttggtactcttgctacgggagacagtctcggaatggagtctccctctgcaacccttgtgaactcctgtgaattccatctttcatccaggactgagatacaaatgtcagagtaaagcaagcaaagatccaagtaaaacgtaattcaagttgacctagttcagaagcagggtgctccttgttaaccttgagccatttatacctttgaaaaatcaaaatcaccgtcagtattttttttttttctttttaactctaaactttgaattaggatatttctatttggc

SEQ ID NO: 24 - protein, 482 aa, Sus scrofa
MRFTLTRWCLTLFIFLNRPLPVLPDSADGAHTPTLEPEPFLYILGKQRMLEAQHRCYDRMQKLPPYQGEGLYCNRTWDGWSCWDDTPAGVLAEQYCPDYFPDFDAAEKVTKYCGEDGDWYRHPESNISWSNYTMCNAFTPDKLQNAYILYYLAIVGHSLSILTLLISLGIFMFLRSISCQRVTLHKNMFLTYVLNSIIIIVHLVVIVPNGELVKRDPPICKVLHFFHQYMMSCNYFWMLCEGVYLHTLIVVSVFAEGQRLWWYHVLGWGFPLIPTTAHAITRAVLFNDNCWLSVDTNLLYIIHGPVMAALVVNFFFLLNILRVLVKKLKESQEAESHMYLKAVRATLILVPLLGVQFVVLPWRPSTPLLGKIYDYVVHSLIHFQGFFVAIIYCFCNHEVQGALKRQWNQYQAQRWAGRRSTRAANAAAATAAAAAALAETVEIPVYICHQEPREEPAGEEPVVEVEGVEVIAMEVLEQETSA

SEQ ID NO: 25 - DNA, 3410 bp, Rattus norvegicus
  CDS (297)..(1733)
ggcacgaggcagaggcaggctctgcaaggagagccagtggcccaagcccctggacggactgcagcttgcagacaactcctggttggagaggtgcttctgctcactctgaggttggcaccccctgcccagtgaagtctgagttcctgagacatccagctagagaattcctgcgtccacctaaggtaagtgccattccacacaagaggaaaccgagggagcacggcttctgaacagcggatctgaaaatgactccacggagatccagaatgaaaaggcggaacctccgaaaaccgaagatgaggttccttctcctgaacaggttcaccctgctgctcctgctcctagtgagccctactccagttcttcaggctcctaccaatctcactgactccggccttgatcaggagccattcctgtacttggttggacgcaagaagcttctggatgctcagtacaagtgctatgaccggattcagcagttgcccccttatgagggagaaggtccatattgcaaccgaacttgggatggatggatgtgctgggacgacactccagctggtgtaatgtcctatcagcactgccctgactactttccggactttgacccaacagaaaaggtttcaaaatactgcgatgagaatggagagtggtttagacaccctgacagcaaccgaacctggtccaactacactctgtgcaatgccttcactcctgacaaactgcataatgcatacgtttcttattacctagcacttgtgggccactccatgtcaattgctgctctgattgcttccatggggatcttcttgtttttcaagaaccttagctgccagagagtgactctgcacaagaacatgttcctcacttatatcctgaactctatcattatcatcatccacctggttgaggttgtgcccaatggagacctggtgcgacgggatcctataagttgcaagattctgcactttttccatcagtacatgatggcttgcaactacttctggatgctgtgtgaggggatctatcttcacactctgatcgtcatggctgtgttcaccgaggatcaacgtctgcgctggtactatttacttggctgggggttcccaatagtgccaactattatccatgccattactcgtgccgtctactacaacgacaactgttggctgagtacggagacccacttgctttacatcatccatggacccgtcatggcggctctggtggtcaacttcttctttctgctcaacattgtccgtgtgcttgtgaccaagatgaggcaaacccatgaagccgaggcctacatgtacctgaaggctgtaaaggccaccatggtccttgtgcccctgctggggatccagtttgttgtgtttccctggaggccctccaacaaagtgcttgggaagatctatgattatctcatgcactctctgattcatttccagggattctttgtcgcgactatctactgcttctgtaaccatgaggtgcaagtgaccctgaagcgtcagtgggcgcagttcaagatccagtggagccatcgctggggtaggcgccggcccaccaaccgagtggttagcgctcctcgtgctgtagcctttgcagaaccaggtggccgccccatttacatctgccatcaggaaccaaggaatcctccagtcagcaacaacgaaggcgaagagggtactgaaatgatccccatgaatgtcatccagcaagactcatccgcttgaatgcttgaatgtgaagcaacccccccaacattgtgatccactgagccttcatttcctgggagaacgatagaccatgcgtttcaagtgatccccatcctcccaggagctaagcatatatttgtgaagatgtattaagtgaatttgtccatagtgaatttgaagaacgttattcttggtactattgctttgggagtcaggctaggctctcagtgcaacttgtgaactccattattcatctaggactgagatgacagtgttcataggaatgtagacaaggtatccaaaactgccactaaattgacctagttcagataagggtgctccatgttaatgttgagccatttgtacctttgagaaactaaaatcactctcaatgtttttaattttaacactggactttgaattagactatttctgtatttggctacggatctggtttttaatgtttttatttcagtcaattcctatattgcacattttttcatccatacaatgtcaactgcacaaataacatgacctctggaagatgctgtgtcttttataacatgatccacgtcaccaggcagacccaaatactgtaaagaattgatctgaatgttgagtgtgctttggtcactgacatttataaattgggaggtcatcaagaacctatcactaaatttttcacaaaactgacccccctcaaatgtaattttccatcaaagagaacactctttaaagatagttttccactttcctaaactacaggatttataaagcaaatcattccaaggtttataaagcagattacctcttgcccttgggtgctatttagcagtgagggatacatttgtttacgactggtaattaaaagactccattcaagtccattaactgccttccacccagcttcaaagcttgacaagatcccgtgttttccaggaagactcagtgtggctaattacaaatcagtttctagttgacctcttgtttgcttctattagcaaaacaaggagggaaaaaagcaaaccacaatgtaaggacttggagtttaaccacatattagttcatcttaaattaaagacaagcttctcaccaaaactcagtaccatatttccacgcctctctttactccccagaatgagatcttccaagatccacaaataaaccaataccattaccaatttctgaatctgcaactgcaatcaattatcattattaatttgtatgctgaatgagacgcatccttgagacccctaaaaatctcatctcctctttgtcatcttgtgtcactgcctttcagaggtgatttagttgcgtaaaaacaaaagtaacttgatttgttctggttacgtacttggtgcacccagagaaaattgtatttcgctagcagctgtggcccttggatactgagagtctcctttaccaaaatgggagaattgaaaggaaggattttttttccaatcacagtgttacgtggtgatgttcccatgttttgtttacaaacattgaaatcagtgtatttcaggcagctctggtacaaatgtgatagtgtattgctgaagtgtttgagacgttattgtgcttctgtagtaggggctgatgaaatcaaactgacttatcatagcattgcatgtaatacctaaattgtgtgaaacacttcttctgtgtacttgtataaattaatacaagcatgtt

SEQ ID NO: 26 - protein, 478 aa, Rattus norvegicus
MRFLLLNRFTLLLLLLVSPTPVLQAPTNLTDSGLDQEPFLYLVGRKKLLDAQYKCYDRIQQLPPYEGEGPYCNRTWDGWMCWDDTPAGVMSYQHCPDYFPDFDPTEKVSKYCDENGEWFRHPDSNRTWSNYTLCNAFTPDKLHNAYVSYYLALVGHSMSIAALIASMGIFLFFKNLSCQRVTLHKNMFLTYILNSIIIIIHLVEVVPNGDLVRRDPISCKILHFFHQYMMACNYFWMLCEGIYLHTLIVMAVFTEDQRLRWYYLLGWGFPIVPTIIHAITRAVYYNDNCWLSTETHLLYIIHGPVMAALVVNFFFLLNIVRVLVTKMRQTHEAEAYMYLKAVKATMVLVPLLGIQFVVFPWRPSNKVLGKIYDYLMHSLIHFQGFFVATIYCFCNHEVQVTLKRQWAQFKIQWSHRWGRRRPTNRVVSAPRAVAFAEPGGRPIYICHQEPRNPPVSNNEGEEGTEMIPMNVIQQDSSA

SEQ ID NO: 27 - DNA, 3382 bp, Rattus norvegicus
  CDS (242)..(1681)
ctgcagcttgcagacaactcctggttggagaggtgcttctgcacctcactctgaggttggcaccccctgcccagtgaagtctgagttcctgagacatccagctagagaattcctgcgtccacctaaggtaagtgccattccacacaagaggaaaccgagggagcacggcttctgaacagcggatctgaaaatgactccacggagatccagaatgaaaaggcggaacctccgaaaaccgaagatgaggttccttctcctgaacaggttcaccctgctgctcctgctcctagtgagccctactccagttcttcaggctcctaccaatctcactgactccggccttgatcaggagccattcctgtacttggttggacgcaagaagcttctggatgctcagtacaagtgctatgaccggattcagcagttgcccccttatgagggagaaggtccatattgcaaccgaacttgggatggatggatgtgctgggacgacactccagctggtgtaatgtcctatcagcactgccctgactactttccggactttgacccaacagaaaaggtttcaaaatactgcgatgaaaatggagagtggtttagacaccctgacagcaaccgaacctggtccaactacactctgtgcaatgccttcactcctgacaaactgcataatgcatacgttctttattacctagcacttgtgggccactccatgtcaattgctgctctgattgcttccatggggatcttcttgtttttcaagaaccttagctgccagagagtgactctgcacaagaacatgttcctcacttatatcctgaactctatcattatcatcatccacctggttgaggttgtgcccaatggagacctggtgcgacgggatcctataagttgcaagattctgcactttttccatcagtacatgatggcttgcaactacttctggatgctgtgtgaggggatctatcttcacactctgatcgtcatggctgtgttcaccgaggatcaacgtctgcgctggtactatttacttggctgggggttcccaatagtgccaactattatccatgccattactcgtgccgtctactacaacgacaactgttggctgagtacggagacccacttgctttacatcatccatggacccgtcatggcggctctggtggtcaacttcttctttctgctcaacattgtccgtgtgcttgtgaccaagatgaggcaaacccatgaagccgaggcctacatgtacctgaaggctgtaaaggccaccatggtccttgtgcccctgctggggatccagtttgttgtgtttccctggaggccctccaacaaagtgcttgggaagatctatgattatctcatgcactctctgattcatttccagggattctttgtcgcgactatctactgcttctgtaaccatgaggtgcaagtgaccctgaagcgtcagtgggcgcagttcaagatccagtggagccatcgctggggtaggcgccgccgccccaccaaccgagtggttagcgctcctcgtgctgtagcctttgcagaaccaggtggcctccccatttacatctgccatcaggaaccaaggaatcctccagtcagcaacaacgaaggcgaagagggtactgaaatgatccccatgaatgtcatccagcaagactcatccgcttgaatgcttgaatgtgaagcaaccccccaacattgtgatccactgagccttcatttcctgggagaacgatagaccatgcgtttcaagtgatccccatcctcccaggagctaagcatatcatttgtgaagatgtattaagtgaatttgtccatagtgaatttgaagaacgttattcttggtactattgctttgggagtcaggctaggctctcagtgcaacttgtgaactccattattcatctaggactgagatgacagtgttcataggaatgtagacaaggtatccaaaactgccactaaattgacctagttcagataagggtgctccatgttaatcttgagccatttgtacctttgagaaactaaaatcactctcaatgtttttaattttaacactggactttgaattagactatttctgtatttggctacggatctggtttttaatgtttttatttcagtcaattcctatattgcacattttttcatccatacaatgtcaactgcacaaataacatgacctctggaagatgctgtgtcttttataacatgatccacgtcaccaggcagacccaaatactgtaaagaattgatctgaatgttgagtgtgctttggtcactgacatttataaattgggaggtcatcaagaacctatcactaaatttttcacaaaactgacccccctcaaatgtaattttccatcaaagagaacactctttaaagatagttttccactttcctaaactacaggatttataaagcaaatcattccaaggtttataaagcagattacctcttgcccttgggtgctatttagcagtgagggatagatttgtttacgactggtaattaaaagactccattcaagtccattaactgccttccacccagcttcaaagcttgacaagatcccgtgttttccaggaagactcagtgtggctaattacaaatcagtttctagttgacctcttgtttgcttctattagcaaaacaaggagggaaaaaagcaaaccacaatgtaaggacttggagtttaaccacatattagttcatcttaaattaaagacaagcttctcaccaaaactcagtaccatatttccacgcctctctttactccccagaatgagatcttccaagatccacaaataaaccaataccattaccaatttctgaatctgcaactgcaatcaattatcattattaatttgtatgctgaatgagacgcatccttgagacccttaaaaatctcatctcctctttgtcatcttgtgtcactgcctttcagaggtgatttagttgcgtaaaaacaaaagtaacttgatttgttctggttacgtacttggtgcacccagagaaaattgtatttcgctagcagctgtggcccttggatactgagagtctcctttaccaaaatgggagaattgaaaggaaggattttttttccaatcacagtgttacgtggtgatgttcccatgttttgtttacaaacattgaaatcagtgtatttcaggcagctctggtacaaatgtgatagtgtattgctgaagtgtttgagacgttattgtgcttctgtagtaggggctgatgaaatcaaactgacttatcatagcattgcatgtaatacctaaattgtgtgaaacacttcttctgtgtacttgtataaattaatacaagcatgttgaaaaggcaaaaaaaaaaaaaaaa

SEQ ID NO: 28 - protein, 479 aa, Rattus norvegicus
MRFLLLNRFTLLLLLLVSPTPVLQAPTNLTDSGLDQEPFLYLVGRKKLLDAQYKCYDRIQQLPPYEGEGPYCNRTWDGWMCWDDTPAGVMSYQHCPDYFPDFDPTEKVSKYCDENGEWFRHPDSNRTWSNYTLCNAFTPDKLHNAYVLYYLALVGHSMSIAALIASMGIFLFFKNLSCQRVTLHKNMFLTYILNSIIIIIHLVEVVPNGDLVRRDPISCKILHFFHQYMMACNYFWMLCEGIYLHTLIVMAVFTEDQRLRWYYLLGWGFPIVPTIIHAITRAVYYNDNCWLSTETHLLYIIHGPVMAALVVNFFFLLNIVRVLVTKMRQTHEAEAYMYLKAVKATMVLVPLLGIQFVVFPWRPSNKVLGKIYDYLMHSLIHFQGFFVATIYCFCNHEVQVTLKRQWAQFKIQWSHRWGRRRRPTNRVVSAPRAVAFAEPGGLPIYICHQEPRNPPVSNNEGEEGTEMIPMNVIQQDSSA

SEQ ID NO: 29 - DNA, 3282 bp, Rattus norvegicus
  CDS (73)..(1620)
cggcacgaggcggatctgaaaatgactcctaggagatccagaatgaaaaggcggaacctccgaaaaccgaagatgaggttccttctcctgaacaggttcaccctgctgctcctgctcctagtgagccctactccagttcttcaggctcctaccaatctcactgactccggccttgatcaggagccattcctgtacttggttggacgcaagaagcttctggatgctcagtacaagtgctatgaccggattcagcagttgcccccttatgagggagaaggtccatattgcaaccgaacttgggatggatggatgtgctgggacgacactccagctggtgtaatgtcctatcagcactgccctgactactttccggactttgacccaacagaaaaggtttcaaaatactgcgatgagaatggagagtggtttagacaccctgacagcaaccgaacctggtccaactacactctgtgcaatgccttcactcctgacaaactgcataatgcatacgtttcttattacctagcacttgtgggccactccatgtcaattgctgctctgattgcttccatggggatcttcttgtttttcaagaaccttagctgccagagagtgactctgcacaagaacatgttcctcacttatatcctgaactctatcattatcatcatccacctggttgaggttgtgcccaatggagacctggtgcgacgggatcctatgcatatttttcatcataacacatatatgtggacaatgcagtgggaactgtcaccacccttacccctgagtgcacacgagggaaagatggaccctcatgacagtgaagtgataagttgcaagattctgcactttttccatcagtacatgatggcttgcaactacttctggatgctgtgtgaggggatctatcttcacactctgatcgtcatggctgtgttcaccgaggatcaacgtctgcgctggtactatttacttggctgggggttcccaatagtgccaactattatccatgccattactcgtgccgtctactacaacgacaactgttggctgagtacggagacccacttgctttacatcatccatggacccgtcatggcggctctggtggtcaacttcttctttctgctcaacattgtccgtgtgcttgtgaccaagatgaggcaaacccatgaagccgaggcctacatgtacctgaaggctgtaaaggccaccatggtccttgtgcccctgctggggatccagtttgttgtgtttccctggaggccctccaacaaagtgcttgggaagatctatgattatctcatgcactctctgattcatttccagggattctttgtcgcgactatctactgcttctgtaaccatgaggtgcaagtgaccctgaagcgtcagtgggcgcagttcaagatccagtggagccatcgctggggtaggcgccggcccaccaaccgagtggttagcgctcctcgtgctgtagcctttgcagaaccaggtggccgccccatttacatctgccatcaggaaccaaggaatcctccagtcagcaacaacgaaggcgaagagggtactgaaatgatccccatgaatgtcatccagcaagactcatccgcttgaatgcttgaatgtgaagcaacccccccaacattgtgatccactgagccttcatttcctgggagaacgatagaccatgcgtttcaagtgatccccatcctcccaggagctaagcatatatttgtgaagatgtattaagtgaatttgtccatagtgaatttgaagaacgttattcttggtactattgctttgggagtcaggctaggctctcagtgcaacttgtgaactccattattcatctaggactgagatgacagtgttcataggaatgtagacaaggtatccaaaactgccactaaattgacctagttcagataagggtgctccatgttaatgttgagccatttgtacctttgagaaactaaaatcactctcaatgtttttaattttaacactggactttgaattagactatttctgtatttggctacggatctggtttttaatgtttttatttcagtcaattcctatattgcacattttttcatccatacaatgtcaactgcacaaataacatgacctctggaagatgctgtgtcttttataacatgatccacgtcaccaggcagacccaaatactgtaaagaattgatctgaatgttgagtgtgctttggtcactgacatttataaattgggaggtcatcaagaacctatcactaaatttttcacaaaactgacccccctcaaatgtaattttccatcaaagagaacactctttaaagatagttttccactttcctaaactacaggatttataaagcaaatcattccaaggtttataaagcagattacctcttgcccttgggtgctatttagcagtgagggatacatttgtttacgactggtaattaaaagactccattcaagtccattaactgccttccacccagcttcaaagcttgacaagatcccgtgttttccaggaagactcagtgtggctaattacaaatcagtttctagttgacctcttgtttgcttctattagcaaaacaaggagggaaaaaagcaaaccacaatgtaaggacttggagtttaaccacatattagttcatcttaaattaaagacaagcttctcaccaaaactcagtaccatatttccacgcctctctttactccccagaatgagatcttccaagatccacaaataaaccaataccattaccaatttctgaatctgcaactgcaatcaattatcattattaatttgtatgctgaatgagacgcatccttgagacccctaaaaatctcatctcctctttgtcatcttgtgtcactgcctttcagaggtgatttagttgcgtaaaaacaaaagtaacttgatttgttctggttacgtacttggtgcacccagagaaaattgtatttcgctagcagctgtggcccttggatactgagagtctcctttaccaaaatgggagaattgaaaggaaggatttttttttccaatcacagtgttacgtggtgatgttcccatgttttgtttacaaacattgaaatcagtgtatttcaggcagctctggtacaaatgtgatagtgtattgctgaagtgtttgagacgttattgtgcttctgtagtaggggctgatgaaatcaaactgacttatcatagcattgcatgtaatacctaaattgtgtgaaacacttcttctgtgtgtagtcgtgcc

SEQ ID NO: 30 - protein, 515 aa, Rattus norvegicus
MRFLLLNRFTLLLLLLVSPTPVLQAPTNLTDSGLDQEPFLYLVGRKKLLDAQYKCYDRIQQLPPYEGEGPYCNRTWDGWMCWDDTPAGVMSYQHCPDYFPDFDPTEKVSKYCDENGEWFRHPDSNRTWSNYTLCNAFTPDKLHNAYVSYYLALVGHSMSIAALIASMGIFLFFKNLSCQRVTLHKNMFLTYILNSIIIIIHLVEVVPNGDLVRRDPMHIFHHNTYMWTMQWELSPPLPLSAHEGKMDPHDSEVISCKILHFFHQYMMACNYFWMLCEGIYLHTLIVMAVFTEDQRLRWYYLLGWGFPIVPTIIHAITRAVYYNDNCWLSTETHLLYIIHGPVMAALVVNFFFLLNIVRVLVTKMRQTHEAEAYMYLKAVKATMVLVPLLGIQFVVFPWRPSNKVLGKIYDYLMHSLIHFQGFFVATIYCFCNHEVQVTLKRQWAQFKIQWSHRWGRRRPTNRVVSAPRAVAFAEPGGRPIYICHQEPRNPPVSNNEGEEGTEMIPMNVIQQDSSA

SEQ ID NO: 31 - DNA, 1808 bp, Rattus norvegicus
  CDS (242)..(1792)
ctgcagcttgcagacaactcctggttggagaggtgcttctgcacctcactctgaggttggcaccccctgcccagtgaagtctgagttcctgagacatccagctagagaattcctgcgtccacctaaggtaagtgccattccacacaagaggaaaccgagggagcacggcttctgaacagcggatctgaaaatgactccacggagatccagaatgaaaaggcggaacctccgaaaaccgaagatgaggttccttctcctgaacaggttcaccctgctgctcctgctcctagtgagccctactccagttcttcaggctcctaccaatctcactgactccggccttgatcaggagccattcctgtacttggttggacgcaagaagcttctggatgctcagtacaagtgctatgaccggattcagcagttgcccccttatgagggagaaggtccatattgcaaccgaacttgggatggatggatgtgctgggacgacactccagctggtgtaatgtcctatcagcactgccctgactactttccggactttgacccaacagaaaaggtttcaaaatactgcgatgaaaatggagagtggtttagacaccctgacagcaaccgaacctggtccaactacactctgtgcaatgccttcactcctgacaaactgcataatgcatacgttctttattacctagcacttgtgggccactccatgtcaattgctgctctgattgcttccatggggatcttcttgtttttcaagaaccttagctgccagagagtgactctgcacaagaacatgttcctcacttatatcctgaactctatcattatcatcatccacctggttgaggttgtgcccaatggagacctggtgcgacgggatcctatgcatatttttcatcataacacatatatgtggacaatgcagtgggaactgtcaccacccttacccctgagtgcacacgagggaaagatggaccctcatgacagtgaagtgataagttgcaagattctgcactttttccatcagtacatgatggcttgcaactacttctggatgctgtgtgaggggatctatcttcacactctgatcgtcatggctgtgttcaccgaggatcaacgtctgcgctggtactatttacttggctgggggttcccaatagtgccaactattatccatgccattactcgtgccgtctattacaacgacaactgttggctgagtacggagacccacttgctttacatcatccatggacccgtcatggcggctctggtggtcaacttcttcttcctgctcaacattgtccgtgtgcttgtgaccaagatgaggcaaacccatgaagccgaggcctacatgtacctgaaggctgtaaaggccaccatggtccttgtgcccctgctggggatccagtttgttgtgtttccctggaggccctccaacaaagtgcttgggaagatctatgattatctcatgcactctctgattcatttccagggattctttgtcgcgactatctactgcttctgtaaccatgaggtgcaagtgaccctgaagcgtcagtgggcgcagttcaagatccagtggagccatcgctggggtaggcgccgccgccccaccaaccgagtggttagcgctcctcgtgctgtagcctttgcagaaccaggtggcctccccatttacatctgccatcaggaaccaaggaatcctccagtcagcaacaacgaaggcgaagagggtactgaaatgatccccatgaatgtcatccagcaagactcatccgcttgaatgcttgaatgtgaag

SEQ ID NO: 32 - protein, 516 aa, Rattus norvegicus
MRFLLLNRFTLLLLLLVSPTPVLQAPTNLTDSGLDQEPFLYLVGRKKLLDAQYKCYDRIQQLPPYEGEGPYCNRTWDGWMCWDDTPAGVMSYQHCPDYFPDFDPTEKVSKYCDENGEWFRHPDSNRTWSNYTLCNAFTPDKLHNAYVLYYLALVGHSMSIAALIASMGIFLFFKNLSCQRVTLHKNMFLTYILNSIIIIIHLVEVVPNGDLVRRDPMHIFHHNTYMWTMQWELSPPLPLSAHEGKMDPHDSEVISCKILHFFHQYMMACNYFWMLCEGIYLHTLIVMAVFTEDQRLRWYYLLGWGFPIVPTIIHAITRAVYYNDNCWLSTETHLLYIIHGPVMAALVVNFFFLLNIVRVLVTKMRQTHEAEAYMYLKAVKATMVLVPLLGIQFVVFPWRPSNKVLGKIYDYLMHSLIHFQGFFVATIYCFCNHEVQVTLKRQWAQFKIQWSHRWGRRRRPTNRVVSAPRAVAFAEPGGLPIYICHQEPRNPPVSNNEGEEGTEMIPMNVIQQDSSA

SEQ ID NO: 33 - DNA, 399 bp, Rattus norvegicus
  CDS (1)..(330)
ccggactttgacccaacagaaaaggtttcaaaatactgcgatgaaaatggagagtggtttagacaccctgacagcaaccgaacctggtccaactacactctgtgcaatgccttcactcctgacaaactgcataatgcatacgttctttattacctagcacttgtgggccactccatgtcaattgctgctctgattgcttccatggggatcttcttgtttttcaagaaccttagctgccagagagtgactctgcacaagaacatgttcctcacttatatcctgaactctatcattatcatcatccacctggttgaggttgtgcccaatggagacctggtgcgacgggatcctataagttgcaagattctgcactttttccatcagtacatgatggcttgc

SEQ ID NO: 34 - protein, 110 aa, Rattus norvegicus
PDFDPTEKVSKYCDENGEWFRHPDSNRTWSNYTLCNAFTPDKLHNAYVLYYLALVGHSMSIAALIASMGIFLFFKNLSCQRVTLHKNMFLTYILNSIIIIIHLVEVVPNG

SEQ ID NO: 35 - DNA, 392 bp, Mus musculus
  CDS (3)..(392)
tggttgaggttgtgcccaatggagatctggtgcggcgggatcctataagttgcaaggttctacactttttacatcagtacatgatgtcttgcaactacttctggatgctctgcgaggggatctatcttcatactctgattgtcatggctgtgtttaccgacgagcaacgcctacgctggtactatcttcttggctgggggttcccgatagtgccaaccattatccacgccatcactcgtgccctctactacaacgacaactgctggctgagtgcagaaacccacttgctttacatcatccatggacccgtcatggtggctctggtggtcagcttcttctttcttctcaacattgtccgcgtgcttgtgaccaagatgaggcaaacccacgag

SEQ ID NO: 36 - protein, 130 aa, Mus musculus
VEVVPNGDLVRRDPISCKVLHFLHQYMMSCNYFWMLCEGIYLHTLIVMAVFTDEQRLRWYYLLGWGFPIVPTIIHAITRALYYNDNCWLSAETHLLYIIHGPVMVALVVSFFFLLNIVRVLVTKMRQTHE

SEQ ID NO: 37 - DNA, 1488 bp, Mus musculus
  CDS (1)..(1488)
atgactccaaggaggtccagagtgaaaaggcggaatctccgcaaaccgaagatgaggttccttctcgtgaacaggttcaccctgctgctcctgctcctagtgagcccaactccagttcttcaggctcctaccaatctcactgactccggccttgatcaggagccattcctgtacttggttggccgcaagaagctgctggatgctcagtacaaatgctatgaccggattcatcagttgccctcttatgaaggagaaggtctgtactgcaaccgcacctgggatggctggatgtgctgggacgacactcccgctggagccacagcctatcagcactgccctgactacttcccggactttgacacagcagaaaaggtttcaaaatactgtgatgaaaatggagagtggtttagacaccctgacagcaaccgaacctggtccaactatactctgtgcaacgctttcacttctgagaaactgcaaaatgcgtacgttctttattacctggctcttgtgggtcactccttgtcgattgctgctttggttgcttccatgctgatcttctggattttcaagaaccttagctgccagagggtgaccttgcacaagcacatgttccttacttatattctgaattctatcattatcatcatccacctggttgaggttgtgcccaatggagatctggtgcggcgggatcctataagttgcaaggttctacactttttacatcagtacatgatgtcttgcaactacttctggatgctctgcgaggggatctatcttcatactctgattgtcatggctgtgtttaccgacgagcaacgcctacgctggtactatcttcttggctgggggttcccgatagtgccaaccattatccacgccatcactcgtgccctctactacaacgacaactgctggctgagtgcagaaacccacttgctttacatcatccatggacccgtcatggtggctctggtggtcaacttcttctttcttctcaacattgtccgcgtgcttgtgaccaagatgaggcaaacccacgaggccgagtcctacatgtacctgaaggctgtgaaggccaccatggtccttgtgcccctgctggggatccagtttgttgtgtttccctggaggccctccaacaaggtgcttgggaagatctatgattatctcatgcactctctgattcatttccagggattctttgtggcgactatctactgcttctgcaaccatgaggtgcaagtcaccctgaagcgccagtggacgcagttcaagatccagtggagccaacgctggggaaggcgccgccccaccaaccgcgtagttagtgctcctcgggctgtagccttcgctgagccagatggcctccccatttacatctgccatcaggaaccacggaatcctccaatcagcaacaacgaaggcgaggagagtactgaaatgatccccatgaacgtcatccagcaagacgcatccgcttga

SEQ ID NO: 38 - protein, 495 aa, Mus musculus
MTPRRSRVKRRNLRKPKMRFLLVNRFTLLLLLLVSPTPVLQAPTNLTDSGLDQEPFLYLVGRKKLLDAQYKCYDRIHQLPSYEGEGLYCNRTWDGWMCWDDTPAGATAYQHCPDYFPDFDTAEKVSKYCDENGEWFRHPDSNRTWSNYTLCNAFTSEKLQNAYVLYYLALVGHSLSIAALVASMLIFWIFKNLSCQRVTLHKHMFLTYILNSIIIIIHLVEVVPNGDLVRRDPISCKVLHFLHQYMMSCNYFWMLCEGIYLHTLIVMAVFTDEQRLRWYYLLGWGFPIVPTIIHAITRALYYNDNCWLSAETHLLYIIHGPVMVALVVNFFFLLNIVRVLVTKMRQTHEAESYMYLKAVKATMVLVPLLGIQFVVFPWRPSNKVLGKIYDYLMHSLIHFQGFFVATIYCFCNHEVQVTLKRQWTQFKIQWSQRWGRRRPTNRVVSAPRAVAFAEPDGLPIYICHQEPRNPPISNNEGEESTEMIPMNVIQQDASA

SEQ ID NO: 39 - DNA, 1437 bp, Mus musculus
  CDS (1)..(1437)
atgaggttccttctcgtgaacaggttcaccctgctgctcctgctcctagtgagcccaactccagttcttcaggctcctaccaatctcactgactccggccttgatcaggagccattcctgtacttggttggccgcaagaagctgctggatgctcagtacaaatgctatgaccggattcatcagttgccctcttatgaaggagaaggtctgtactgcaaccgcacctgggatggctggatgtgctgggacgacactcccgctggagccacagcctatcagcactgccctgactacttcccggactttgacacagcagaaaaggtttcaaaatactgtgatgaaaatggagagtggtttagacaccctgacagcaaccgaacctggtccaactatactctgtgcaacgctttcacttctgagaaactgcaaaatgcgtacgttctttattacctggctcttgtgggtcactccttgtcgattgctgctttggttgcttccatgctgatcttctggattttcaagaaccttagctgccagagggtgaccttgcacaagcacatgttccttacttatattctgaattctatcattatcatcatccacctggttgaggttgtgcccaatggagatctggtgcggcgggatcctataagttgcaaggttctacactttttacatcagtacatgatgtcttgcaactacttctggatgctctgcgaggggatctatcttcatactctgattgtcatggctgtgtttaccgacgagcaacgcctacgctggtactatcttcttggctgggggttcccgatagtgccaaccattatccacgccatcactcgtgccctctactacaacgacaactgctggctgagtgcagaaacccacttgctttacatcatccatggacccgtcatggtggctctggtggtcaacttcttctttcttctcaacattgtccgcgtgcttgtgaccaagatgaggcaaacccacgaggccgagtcctacatgtacctgaaggctgtgaaggccaccatggtccttgtgcccctgctggggatccagtttgttgtgtttccctggaggccctccaacaaggtgcttgggaagatctatgattatctcatgcactctctgattcatttccagggattctttgtggcgactatctactgcttctgcaaccatgaggtgcaagtcaccctgaagcgccagtggacgcagttcaagatccagtggagccaacgctggggaaggcgccgccccaccaaccgcgtagttagtgctcctcgggctgtagccttcgctgagccagatggcctccccatttacatctgccatcaggaaccacggaatcctccaatcagcaacaacgaaggcgaggagagtactgaaatgatccccatgaacgtcatccagcaagacgcatccgcttga

SEQ ID NO: 40 - protein, 478 aa, Mus musculus
MRFLLVNRFTLLLLLLVSPTPVLQAPTNLTDSGLDQEPFLYLVGRKKLLDAQYKCYDRIHQLPSYEGEGLYCNRTWDGWMCWDDTPAGATAYQHCPDYFPDFDTAEKVSKYCDENGEWFRHPDSNRTWSNYTLCNAFTSEKLQNAYVLYYLALVGHSLSIAALVASMLIFWIFKNLSCQRVTLHKHMFLTYILNSIIIIIHLVEVVPNGDLVRRDPISCKVLHFLHQYMMSCNYFWMLCEGIYLHTLIVMAVFTDEQRLRWYYLLGWGFPIVPTIIHAITRALYYNDNCWLSAETHLLYIIHGPVMVALVVNFFFLLNIVRVLVTKMRQTHEAESYMYLKAVKATMVLVPLLGIQFVVFPWRPSNKVLGKIYDYLMHSLIHFQGFFVATIYCFCNHEVQVTLKRQWTQFKIQWSQRWGRRRPTNRVVSAPRAVAFAEPDGLPIYICHQEPRNPPISNNEGEESTEMIPMNVIQQDASA

SEQ ID NO: 41 - DNA, 3731 bp, Mus musculus
  CDS (508)..(2055)
aattcggcacgaggctgcacccagctccggataggaggtggaggatagcccgtccttggaagcaacttgctttcctcccagggtgcgcctgccagcccagactgcgcccgcctggaatcagattgtccgggctcctgctggctttcagtggagaaggggaattgtcctcaacaccaagtcaggagagccagccgccccaaagactctgggcgactgcacctggctggcttgcagacaactcttggttggagaggtgcttacttctgcgcctcacgctgccgttggccatccctgcctgcagatgcccagtgaaggtctgctttcctgagaacacctgagctgtgcccagacatccagcaagagaattcctgcatccacctaaggtaagtgccattagagcgccttccagaggagaagaaaccgagggagcagggctactacacagaggatttgaaaatgactccaaggaggtccagagtgaaaaggcggaatctccgcaaaccgaagatgaggttccttctcgtgaacaggttcaccctgctgctcctgctcctagtgagcccaactccagttcttcaggctcctaccaatctcactgactccggccttgatcaggagccattcctgtacttggttggccgcaagaagctgctggatgctcagtacaaatgctatgaccggattcatcagttgccctcttatgaaggagaaggtctgtactgcaaccgcacctgggatggctggatgtgctgggacgacactcccgctggagccacagcctatcagcactgccctgactacttcccggactttgacacagcagaaaaggtttcaaaatactgtgatgaaaatggagagtggtttagacaccctgacagcaaccgaacctggtccaactatactctgtgcaacgctttcacttctgagaaactgcaaaatgcgtacgttctttattacctggctcttgtgggtcactccttgtcgattgctgctttggttgcttccatgctgatcttctggattttcaagaaccttagctgccagagggtgaccttgcacaagcacatgttccttacttatattctgaattctatcattatcatcatccacctggttgaggttgtgcccaatggagatctggtgcggcgggatcctatgcatatttttcatcataacacacatatgtggacaatgcagtgggaactgtcaccacccttacccctgtgtgcacacgagggaaagatggaccctcatgccagtgaagtgataagttgcaaggttctacactttttacatcagtacatgatgtcttgcaactacttctggatgctctgcgaggggatctatcttcatactctgattgtcatggctgtgtttaccgacgagcaacgcctacgctggtactatcttcttggctgggggttcccgatagtgccaaccattatccacgccatcactcgtgccctctactacaacgacaactgctggctgagtgcagaaacccacttgctttacatcatccatggacccgtcatggtggctctggtggtcaacttcttctttcttctcaacattgtccgcgtgcttgtgaccaagatgaggcaaacccacgaggccgagtcctacatgtacctgaaggctgtgaaggccaccatggtccttgtgcccctgctggggatccagtttgttgtgtttccctggaggccctccaacaaggtgcttgggaagatctatgattatctcatgcactctctgattcatttccagggattctttgtggcgactatctactgcttctgcaaccatgaggtgcaagtcaccctgaagcgccagtggacgcagttcaagatccagtggagccaacgctggggaaggcgccgccccaccaaccgcgtagttagtgctcctcgggctgtagccttcgctgagccagatggcctccccatttacatctgccatcaggaaccacggaatcctccaatcagcaacaacgaaggcgaggagagtactgaaatgatccccatgaacgtcatccagcaagacgcatccgcttgaatgtgaagccaccccaagcattgtgatccactgagccttcatttcctggggaaagacagaccatgtgtttcaagtgattcccatcctcccaggagctgaccatatcatttgtgaagaagtgttaagtgaatttgtccatagtgaatttgaagaaagtgattcttggtactattgctttgggagtcagtctaggaatagagtctcccattgcaacttgtgaactccatcattcatcctggactgagatgactgtgttcgtaggaaagcaggcaaggtgttcaaaagatgtcacactattgacctagttcagatacagggtgctcccttgtgaaattttgagccatttgtaccgttgagaaattaaaatcactctcaatgtttttaaatttgacactggattttgaattagactatttctgtatttgactacagatctggtttttaatgtttttatttcagtcagttcctatattacacatgttaccatccatacaatgtcaactacacatatagcatgacctctgaaagatggtgtgtcttttataacatgatccagttcaccaggcagacccaaagtgctgtaatgaattgatctgaaccttgtatgtgctttggtcactgacatttataagttgggaggtcaccaagaacctatcaccaaatttatcacaaaactgccctccaaaatgtaacaatctttaaagagagttttcacttttcctaaactacaaggaatttagtcaagagaccagagtcactccaaggattataaaagcaagattaacctcttgcccttgggtgctatctagcagtaagagaagatttgtttacgaactggtaattaaagactcctatacaagtccattaactgccttccacccagcttcaaagctaaacaagatccctgtgttttcaagaagactctgtgtggctaattacaaatcagtttcttgttgacctcttgcttgctgctattagcaaaacaagagggggaaaaaacccacaatgtagggacttcaaatttaacctcatattaattcatcttaaattaaaaacagagcttctcatcaaaacccagtactatatctccatacccatctctttactccccagtaggagatcatctaagatccatgaataaaccaataccatcacagatttctgaatctgcaacagtaaccagtcatcattacttaatttgtatgctaaatgaggagcttttttgagaccctcaaaatctcctgctcacctttgtaatcttttgtcactgcctttcagaggtgatttagttgtgtaaagacaaaagtaacttgatttgttctggttacatgcttggtgtacccagagaaactgtatttcaatagcaactgtggaccttggatactgagagttgcctttaccaaaatggaaggacggattgaaggaaggatgttttttcaatcacagtgttacctggtgatgtccctatgttttgtttacacacatcgaaatcagtgtatttcaggcagctctagtacaaatgtgataatatattgaagtgtttgagatgttattgtgcttatgtagtaggagctgatgaaatcaaactgacttaccataacattgcatgtcatacctaaattgtgggaaacgcttctgctgtgtacatgtataaatacagagcatattaaaaaggc

SEQ ID NO: 42 - protein, 515 aa, Mus musculus
MRFLLVNRFTLLLLLLVSPTPVLQAPTNLTDSGLDQEPFLYLVGRKKLLDAQYKCYDRIHQLPSYEGEGLYCNRTWDGWMCWDDTPAGATAYQHCPDYFPDFDTAEKVSKYCDENGEWFRHPDSNRTWSNYTLCNAFTSEKLQNAYVLYYLALVGHSLSIAALVASMLIFWIFKNLSCQRVTLHKHMFLTYILNSIIIIIHLVEVVPNGDLVRRDPMHIFHHNTHMWTMQWELSPPLPLCAHEGKMDPHASEVISCKVLHFLHQYMMSCNYFWMLCEGIYLHTLIVMAVFTDEQRLRWYYLLGWGFPIVPTIIHAITRALYYNDNCWLSAETHLLYIIHGPVMVALVVNFFFLLNIVRVLVTKMRQTHEAESYMYLKAVKATMVLVPLLGIQFVVFPWRPSNKVLGKIYDYLMHSLIHFQGFFVATIYCFCNHEVQVTLKRQWTQFKIQWSQRWGRRRPTNRVVSAPRAVAFAEPDGLPIYICHQEPRNPPISNNEGEESTEMIPMNVIQQDASA

SEQ ID NO: 43 - DNA, 1548 bp, Mus musculus
  CDS (1)..(1548)
atgaggttccttctcgtgaacaggttcaccctgctgctcctgctcctagtgagcccaactccagttcttcaggctcctaccaatctcactgactccggccttgatcaggagccattcctgtacttggttggccgcaagaagctgctggatgctcagtacaaatgctatgaccggattcatcagttgccctcttatgaaggagaaggtctgtactgcaaccgcacctgggatggctggatgtgctgggacgacactcccgctggagccacagcctatcagcactgccctgactacttcccggactttgacacagcagaaaaggtttcaaaatactgtgatgaaaatggagagtggtttagacaccctgacagcaaccgaacctggtccaactatactctgtgcaacgctttcacttctgagaaactgcaaaatgcgtacgttctttattacctggctcttgtgggtcactccttgtcgattgctgctttggttgcttccatgctgatcttctggattttcaagaaccttagctgccagagggtgaccttgcacaagcacatgttccttacttatattctgaattctatcattatcatcatccacctggttgaggttgtgcccaatggagatctggtgcggcgggatcctatgcatatttttcatcataacacacatatgtggacaatgcagtgggaactgtcaccacccttacccctgtgtgcacacgagggaaagatggaccctcatgccagtgaagtgataagttgcaaggttctacactttttacatcagtacatgatgtcttgcaactacttctggatgctctgcgaggggatctatcttcatactctgattgtcatggctgtgtttaccgacgagcaacgcctacgctggtactatcttcttggctgggggttcccgatagtgccaaccattatccacgccatcactcgtgccctctactacaacgacaactgctggctgagtgcagaaacccacttgctttacatcatccatggacccgtcatggtggctctggtggtcaacttcttctttcttctcaacattgtccgcgtgcttgtgaccaagatgaggcaaacccacgaggccgagtcctacatgtacctgaaggctgtgaaggccaccatggtccttgtgcccctgctggggatccagtttgttgtgtttccctggaggccctccaacaaggtgcttgggaagatctatgattatctcatgcactctctgattcatttccagggattctttgtggcgactatctactgcttctgcaaccatgaggtgcaagtcaccctgaagcgccagtggacgcagttcaagatccagtggagccaacgctggggaaggcgccgccccaccaaccgcgtagttagtgctcctcgggctgtagccttcgctgagccagatggcctccccatttacatctgccatcaggaaccacggaatcctccaatcagcaacaacgaaggcgaggagagtactgaaatgatccccatgaacgtcatccagcaagacgcatccgcttga

SEQ ID NO: 44 - protein, 515 aa, Mus musculus; identical to SEQ ID NO: 42

SEQ ID NO: 45 - DNA, 800 bp, Homo sapiens
  CDS (33)..(479)
cgagcggactcgactcggcaccgctgtgcaccatggcccgggccctgtgccgcctcccgcggcgcggcctctggctgctcctggcccatcacctcttcatgaccactgcctgccaggaggctaactacggtgccctcctccgggagctctgcctcacccagttccaggtagacatggaggccgtcggggagacgctgtggtgtgactggggcaggaccatcaggagctacagggagctggccgactgcacctggcacatggcggagaagctgggctgcttctggcccaatgcagaggtggacaggttcttcctggcagtgcatggccgctacttcaggagctgccccatctcaggcagggccgtgcgggacccgcccggcagcatcctctaccccttcatcgtggtccccatcacggtgaccctgctggtgacggcactggtggtctggcagagcaagcgcactgagggcattgtgtaggcggggcccaggctgcccgcgggtgcacccaggctgcagggtgaggccaggcaggcctgggtaggggcagcttctggagccttgggacagagcaggcccacaatgccccccttcttccagccaagaagagctcacaggagtccagagtagccgaggctctggtattaacctggaagcccccctggctggaggccaccgccaccctaggaagggggcagggacgtgaccttgacttacctctggaaagggtcccagcctagactgcttaccccatagccacatttgtggatgagtggtttgtgattaaaagggatgttcttg

SEQ ID NO: 46 - protein, 148 aa, Homo sapiens
MARALCRLPRRGLWLLLAHHLFMTTACQEANYGALLRELCLTQFQVDMEAVGETLWCDWGRTIRSYRELADCTWHMAEKLGCFWPNAEVDRFFLAVHGRYFRSCPISGRAVRDPPGSILYPFIVVPITVTLLVTALVVWQSKRTEGIV

SEQ ID NO: 47 - DNA, 825 bp, Homo sapiens
  CDS (34)..(480)
ggcacgaggctcgactcggcaccgctgtgcaccatggcccgggccctgtgccgcctcccgcggcgcggcctctggctgctcctggcccatcacctcttcatgaccactgcctgccaggaggctaactacggtgccctcctccgggagctctgcctcacccagttccaggtagacatggaggccgtcggggagacgctgtggtgtgactggggcaggaccatcaggagctacagggagctggccgactgcacctggcacatggcggagaagctgggctgcttctggcccaatgcagaggtggacaggttcttcctggcagtgcatggccgctacttcaggagctgccccatctcaggcagggccgtgcgggacccgcccggcagcatcctctaccccttcatcgtggtccccatcacggtgaccctgctggtgacggcactggtggtctggcagagcaagcgcactgagggcattgtgtaggcggggcccaggctgcccgcgggtgcacccaggctgcagggtgaggccaggcaggcctgggtaggggcagcttctggagccttgggacagagcaggcccacaatgccccccttcttccagccaagaagagctcacaggagtccagagtagccgaggctctggtattaacctggaagcccccctggctggaggccaccgccaccctaggaagggggcagggacgtgaccttgacttacctctggaaagggtcccagcctagactgcctaccccatagccacatttgtggatgagtggtttgtgattaaaagggatgttcttgaacttgaaaaaaaaaaaaaaaaaa

SEQ ID NO: 48 - protein, 148 aa, Homo sapiens; identical to SEQ ID NO: 46

SEQ ID NO: 49 - DNA, 549 bp, Rattus norvegicus
  CDS (17)..(463)
cggggctctgcttgccatggcccccggcctgcggggcctcccgcggcgcggcctctggctgctgctggctcatcatctcttcatggtcactgcctgccgggaccctgactatggtactctcatccaggagctgtgtctcagccgcttcaaagaggacatggagaccatagggaagactctgtggtgtgactggggaaagaccatagggagctatggggagctcactcactgcaccaaactcgtggcaaacaagattggctgtttctggcccaatccggaagtggacaagttcttcattgctgtccaccaccgctacttcagcaagtgcccagtctcgggcagggccctgcgggaccctcccaacagcatcctctgccctttcattgtgctccccattacggtcacactgctcatgactgccctggtggtctggaggagcaagcgcacagagggcatcgtgtaggtgtcccaggtgatgggggattggggaaccatgggcctggcccagcaacagcatcagggacagaccccacattgcatcctgctcta

SEQ ID NO: 50 - protein, 148 aa, Rattus norvegicus
MAPGLRGLPRRGLWLLLAHHLFMVTACRDPDYGTLIQELCLSRFKEDMETIGKTLWCDWGKTIGSYGELTHCTKLVANKIGCFWPNPEVDKFFIAVHHRYFSKCPVSGRALRDPPNSILCPFIVLPITVTLLMTALVVWRSKRTEGIV

SEQ ID NO: 51 - DNA, 472 bp, Rattus norvegicus
  CDS (16)..(462)
ggggctctgcttgccatggccctcggcctgcggggcctcccgcggcgcggcctctggctgctgctggttcatcatctcttcatggtcactgcctgccgggaccctgactatggtactctcatccaggagctgtgtctcagccgcttcaaagaggacatggagaccatagggaagactctgtggtgtgactggggaaagaccatagggagctatggggagctcactcactgcaccaaactcgtggcaaacaagattggctgtttctggcccaatccggaagtggacaagttcttcattgctgtccaccaccgctacttcagcaagtgcccagtctcgggcagggccctgcgggaccctcccaacagcatcctctgccctttcattgtgctccccattacggtcacactgctcatgactgccctggtggtctggaggagcaagcgcacagagggcatcgtgtaggtgtcccagg

SEQ ID NO: 52 - protein, 148 aa, Rattus norvegicus
MALGLRGLPRRGLWLLLVHHLFMVTACRDPDYGTLIQELCLSRFKEDMETIGKTLWCDWGKTIGSYGELTHCTKLVANKIGCFWPNPEVDKFFIAVHHRYFSKCPVSGRALRDPPNSILCPFIVLPITVTLLMTALVVWRSKRTEGIV

SEQ ID NO: 53 - DNA, 993 bp, Mus musculus
  CDS (34)..(558)
tgagcggataacaatttcacacaggacacagctatgaccttgattacgccaagctcgaaattaaccctcactaaagggaacaaaagctggagctcgcgcgcctgcaggtcgacactagtggatccaaagaattcggcacgagtggggctctgcttgccatggccccgggcctgcggggcctcccgcggtgcggcctctggctgctgctggctcaccatctcttcatggtcactgcctgccgggaccctgactatgggactctcatccaggagctgtgcctcagccgcttcaaggagaacatggagactattgggaagacgctatggtgtgactggggaaagaccatacagagctatggggagctcacttactgcaccaagcacgtggcgcacacgattggctgtttctggcccaatccggaagtggacagattcttcatcgctgtccaccatcgatacttcagcaagtgccccatctcgggcagggccctgcgggaccctcccaacagcatcctctgccctttcattgcgctccccattacggtcacgctgctcatgactgcactggtggtctggaggagcaagcgcacagagggcatcgtgtaggtatcccaggtgatgagggattggggaaccatgggcctagcccagcaacagcatcaggagcaggtcccaccttgcatcctgctctagcctagttagcacaggacttgagagtggctctgcattaagctgaatatgccctagccagaggcttctagcaggggagcaggggcctgcctctgcttacctctgagattgttctaagcaccaggtctctggtacacttcatcaccactgtgggcattctgaactccaaggaagactgcacagatgtgtttgtagatgcacagtttgtgattaaaagagtattcttaaaaaaaaaaaaaaaaaaaaaaaaaaaaaaaaaaaaaaaaaaaaaaaaaaaaaaaaaaaaaaaaaaaaaaaaaaaaaa

SEQ ID NO: 54 - protein, 174 aa, Mus musculus
MTLITPSSKLTLTKGNKSWSSRACRSTLVDPKNSARVGLCLPWPRACGASRGAASGCCWLTISSWSLPAGTLTMGLSSRSCASAASRRTWRLLGRRYGVTGERPYRAMGSSLTAPSTWRTRLAVSGPIRKWTDSSSLSTIDTSASAPSRAGPCGTLPTASSALSLRSPLRSRCS

SEQ ID NO: 55 - DNA, 844 bp, Mus musculus
  CDS (60)..(506)
ggcacgaggagcgggcgcagagctcggcaaggcgctgggacggtggggctctgcttgccatggccccgggcctgcggggcctcccgcggtgcggcctctggctgctgctggctcaccatctcttcatggtcactgcctgccgggaccctgactatgggactctcatccaggagctgtgcctcagccgcttcaaggagaacatggagactattgggaagacgctatggtgtgactggggaaagaccatacagagctatggggagctcacttactgcaccaagcacgtggcgcacacgattggctgtttctggcccaatccggaagtggacagattcttcatcgctgtccaccatcgatacttcagcaagtgccccatctcgggcagggccctgcgggaccctcccaacagcatcctctgccctttcattgcgctccccattacggtcacgctgctcatgactgcactggtggtctggaggagcaagcgcacagagggcatcgtgtaggtatcccaggtgatgagggattggggaaccatgggcctagcccagcaacagcatcaggagcaggtcccaccttgcatcctgctctagcctagttagcacaggacttgagagtggctctgcattaagctgaatatgccctagccagaggcttctagcaggggagcaggggcctgcctctgcttacctctgagattgttctaagcaccaggtctctggtacacttcatcaccactgtgggcattctgaactccaaggaagactgcacagatgtgtttgtagatgcacagtttgtgattaaaagagtattcttaaacctggaaaaaaaaaaaaaaaaaaaa

SEQ ID NO: 56 - protein, 148 aa, Mus musculus
MAPGLRGLPRCGLWLLLAHHLFMVTACRDPDYGTLIQELCLSRFKENMETIGKTLWCDWGKTIQSYGELTYCTKHVAHTIGCFWPNPEVDRFFIAVHHRYFSKCPISGRALRDPPNSILCPFIALPITVTLLMTALVVWRSKRTEGIV

SEQ ID NO: 57 - DNA, 798 bp, Mus musculus
  CDS (25)..(471)
ccacgcgtccgggctctgcttgccatggccccgggcctgcggggcctcccgcggtgcggcctctggctgctgctggctcaccatctcttcatggtcactgcctgccgggaccctgactatgggactctcatccaggagctgtgcctcagccgcttcaaggagaacatggagactattgggaagacgctatggtgtgactggggaaagaccatacagagctatggggagctcacttactgcaccaagcacgtggcgcacacgattggctgtttctggcccaatccggaagtggacagattcttcatcgctgtccaccatcgatacttcagcaagtgccccatctcgggcagggccctgcgggaccctcccaacagcatcctctgccctttcattgcgctccccattacggtcacgctgctcatgactgcactggtggtctggaggagcaagcgcacagagggcatcgtgtaggtatcccaggtgatgagggattggggaaccatgggcctagcccagcaacagcatcaggagcaggtcccaccttgcatcctgctctagcctagttagcacaggacttgagagtggctctgcattaagctgaatatgccctagccagaggcttctagcaggggagcaggggcctgcctctgcttacctctgagattgttctaagcaccaggtctctggtacacttcatcaccactgtgggcattctgaactccaaggaagactgcacagatgtgtttgtagatgcacagtttgtgattaaaagagtattcttaaaaaaaaaaaaaaaaa

SEQ ID NO: 58 - protein, 148 aa, Mus musculus; identical to SEQ ID NO: 56

SEQ ID NO: 59 - DNA, 240 bp, Sus scrofa
  CDS (1)..(240)
aggaccatcaggagctataaagacctctcagactgcaccaggctcgtggcgcaaaggctggactgcttctggcccaacgcggcggtggacaagttcttcctgggagtccaccagcagtacttcagaaactgccccgtctccggcagggccttgcaggacccgcccagcagcgtcctctgccccttcatcgtcgtccccatcctggcgaccctgctcatgaccgcactggtggtctggcag

SEQ ID NO: 60 - protein, 80 aa, Sus scrofa
RTIRSYKDLSDCTRLVAQRLDCFWPNAAVDKFFLGVHQQYFRNCPVSGRALQDPPSSVLCPFIVVPILATLLMTALVVWQ

SEQ ID NO: 61 - DNA, 1312 bp, Homo sapiens
  CDS (30)..(476)
gagcgtgacccagctgcggccggccagccatggagactggagcgctgcggcgcccgcaacttctcccgttgctgctgctgctctgcggtgggtgtcccagagcaggcggctgcaacgagacaggcatgttggagaggctgcccctgtgtgggaaggctttcgcagacatgatgggcaaggtggacgtctggaagtggtgcaacctgtccgagttcatcgtgtactatgagagtttcaccaactgcaccgagatggaggccaatgtcgtgggctgctactggcccaaccccctggcccagggcttcatcaccggcatccacaggcagttcttctccaactgcaccgtggacagggtccacttggaggaccccccagacgaggttctcatcccgctgatcgttatacccgtcgttctgactgtcgccatggctggcctggtggtgtggcgcagcaaacgcaccgacacgctgctgtgagggtcccggtgagatggagtgggtcacacctggcaagctggaagaaagttccctggggatgggagatcgggtgggtgctgccaatctccagctactgtggccacaccccacctggtcatgggcagacccctcccttcctgggctgacctgctccctcgaggccagcctgctccctggctgaggctcaggctatccgcccaagctctttgctcattctagggccagtggaggaaaatgtgataaggccagagcttgtgtgctgggcaagaaatcacctgctgcatcctgtgctccgcaggctgggccggaagcctctgcctgcaggtttctatgctgtttcttagcacagaatccagcctagccttagccgcagtctaggccctgcttggactaggactccttgcttgaccccatctctggttcctgccctggctcctgcaccagccccagctcctgcctacatccaggcagaaatataggcaggggctcttggaagacgttccgtgctgtgacctccgagccctcctggtgggaagacagctggaaaggctgggaggagaagggaggggctgggggttcccaggagccatgcgtggcctgcagagtccattccatcatgatgctgtgcccgctatgggctgtgtccatgaccagaggctggagtgggggtgtgttatagcccctcaccgggacttgctgtgcggatggggcctgggcctccttcctacaggggctcctctgtgggtgaggggccctctggaatggcatcccatgagcttgtggcctctatctgctaccatctgtgttttatctgagtaaagttaccttacttctgg

SEQ ID NO: 62 - protein, 148 aa, Homo sapiens
METGALRRPQLLPLLLLLCGGCPRAGGCNETGMLERLPLCGKAFADMMGKVDVWKWCNLSEFIVYYESFTNCTEMEANVVGCYWPNPLAQGFITGIHRQFFSNCTVDRVHLEDPPDEVLIPLIVIPVVLTVAMAGLVVWRSKRTDTLL

SEQ ID NO: 63 - DNA, 1191 bp, Mus musculus
  CDS (1)..(444)
atgaagaccccagcacagcggctgcaccttcttccactgttgttgctgctttgtggtgagtgtgcccaggtatgcggctgcaacgagacagggatgctggagaggctgcctcgctgtgggaaagccttcgctgacatgatgcagaaggtggctgtctggaagtggtgcaacctgtcggagttcatcgtgtattatgaaagcttcactaactgcaccgagatggagaccaacatcatgggctgctactggcccaacccgctggcccagagcttcatcactggaatccacaggcagttcttttccaactgcacggtggacaggacccactgggaagaccccccggatgaagtactcatcccactgatcgcggttcctgtcgtgctgactgtggctatggctggcctggtggtgtggcgcagcaagcacactgatcggctgctgtgaggatctgctggatggagggccatgcctggcaggctgggagaatgttgctcagagctctgagagctggcagactcggcttctgtctggtttgctttggccacaccctacctggccatgccaaagtcctcctgaccaggctggtgtggcccttgctgtctagcctgccgcctgctggggttcagattgtccatactttgctctttcttgggctagtggaagaaagtgacaaatcccaagtttgtggaccaggcatggaaatcaactgttgctgagccccgctccccaggctcggttccctagtttctagccgtttcttggcagagtcttgctcagcctgaaccccgccccaggtcctgacccatttctagtcctgaccctgacccctgctacacttggccagagagggcaggcaaggtcatctggaagatgtggacgcccccccgcctctgttcaagagactgagcacatcatttatcagacatgaaggatagcctggggtcattaggagccacgtgtgacctactgacccacctgcctgtcctctctgtgatctgtcacgattctgtgtccagtgtgggctggagctgtggcttgtttagcccttcaaagacacctaccctgcaggtagagcgtgaacctccttcttgaggggtattcctgggagtggggcgcactgagtgtgctcaagggttctgtctgctgatgtcagttctttttgattaaagtgtctccttacacgtggaaaaa

SEQ ID NO: 64 - protein, 147 aa, Mus musculus
MKTPAQRLHLLPLLLLLCGECAQVCGCNETGMLERLPRCGKAFADMMQKVAVWKWCNLSEFIVYYESFTNCTEMETNIMGCYWPNPLAQSFITGIHRQFFSNCTVDRTHWEDPPDEVLIPLIAVPVVLTVAMAGLVVWRSKHTDRLL

SEQ ID NO: 65 - DNA, 634 bp, Rattus norvegicus
  CDS (12)..(455)
cttagctggccatggcgaccccggcacagcggctgcaccttctccctctgttgctgctgctttgtggtgagtgtgcccaagtatgcggttgcaatgagacaggaatgctggagaggttgcctcgctgtgggaaagcctttgccgaaatgatgcagaaggtggacgtctggaagtggtgcaacctgtcggagttcatcgtgtactacgaaagcttcactaactgcacggagatggagacaaacatcgtgggctgctactggcccaacccgctggcccagagctttatcacgggtatccataggcagttcttttccaactgcactgtggacaggacccactgggaagaccccccggatgaagttctcatcccacttatcgcagttccagtcttgctgaccgtggctatggctggcctggtggtgtggcgtagcaagcacactgaccggcttctgtgagggcctgcgggatggaaggccgtgcctggcaagctagaagaatgtcgcccagagctctgagagcctgcagactctgtttctgtctggtctgccttggccacaccctacctggtcgtgtcaaagtcctcctgactagactgacgtggcccttgctgtctagcctgctgcctggtggggtt

SEQ ID NO: 66 - protein, 147 aa, Rattus norvegicus
MATPAQRLHLLPLLLLLCGECAQVCGCNETGMLERLPRCGKAFAEMMQKVDVWKWCNLSEFIVYYESFTNCTEMETNIVGCYWPNPLAQSFITGIHRQFFSNCTVDRTHWEDPPDEVLIPLIAVPVLLTVAMAGLVVWRSKHTDRLL

SEQ ID NO: 67 - DNA, 1218 bp, Rattus norvegicus
  CDS (31)..(474)
tgctgtaagccgagtgcatcttagctggccatggcgaccccagcacagcggctgcaccttctccctctgttgctgctgctttgtggtgagtgtgcccaagtatgcggttgcaatgagacaggaatgctggagaggttgcctcgctgtgggaaagcctttgccgaaatgatgcagaaggtggacgtctggaagtggtgcaacctgtcggagttcatcgtgtactacgaaagcttcactaactgcacggagatggagacaaacatcgtgggctgctactggcccaacccgctggcccagagctttatcacgggtatccataggcagttcttttccaactgcactgtggacaggacccactgggaagaccccccggatgaagttctcatcccacttatcgcagttccagtcttgttgaccgtggctatggctgggctggtggtatggcgtagcaagcgcactgaccggcttctgtgagggcctgcgggatggaaggccgtgcctggcaagctagaagaatgtcgcccagagctctgagagcctgcagactctgtttctgtctggtctgccttggccacaccctacctggtcgtgtcaaagtcctcctgactagactgacgtggcccttgctgtctagcctgctgcctggtggggttcaggctgccccgacaggtgcttcactctttcttgggctagtggaagaaagtgacaaacctcacatttgtgtaccaggcgtggaaatcatctcttgctgagtcccaccctccctgggctgaattcccgagtttctagacatttcttggcagagtctggcccagcctcaaccctaacccaggtcctgacccatttctatccctgaccctgacctctgctacgcttggggcagagagggcaggcaaggtcatctggaaggtgtggcgtcccgagcagaaagagagatgagcacattctctgttggacatgaagggtagtcggggggattggggaatggagttgttaggagtcatgtgtgacctacggacctgcctgtcttctaggcgatctgccatgattctgtgtccagtgtgggctggagctgtgatttgtttaacccttcagacacctaccctgaagatagagagtgaacctccttcctgaggggtattcctgggggtgaggcacgctctgagcgtgctcaagggttctgtctgctgatgtctgctttctgattaaagtgtctcc

SEQ ID NO: 68 - protein, 147 aa, Rattus norvegicus
MATPAQRLHLLPLLLLLCGECAQVCGCNETGMLERLPRCGKAFAEMMQKVDVWKWCNLSEFIVYYESFTNCTEMETNIVGCYWPNPLAQSFITGIHRQFFSNCTVDRTHWEDPPDEVLIPLIAVPVLLTVAMAGLVVWRSKRTDRLL

SEQ ID NO: 69 - DNA, 1234 bp, Rattus norvegicus
  CDS (33)..(476)
gttgctgtaagccgagtgcatcttagctggccatggcgaccccggcacagcggctgcaccttctccctctgttgctgctgctttgtggtgagtgtgcccaagtatgcggttgcaatgagacaggaatgctggagaggttgcctcgctgtgggaaagcctttgccgaaatgatgcagaaggtggacgtctggaagtggtgcaacctgtcggagttcatcgtgtactacgaaagcttcactaactgcacggagatggagacaaacatcgtgggctgctactggcccaacccgctggcccagagctttatcacgggtatccataggcagttcttttccaactgcactgtggacaggacccactgggaagaccccccggatgaagttctcatcccacttatcgcagttccagtcttgttgaccgtggctatggctgggctggtggtatggcgtagcaagcgcactgaccggcttctgtgagggcctgcgggatggaaggccgtgcctggcaagctagaagaatgtcgcccagagctctgagagcctgcagactctgtttctgtctggtctgccttggccacaccctacctggtcgtgtcaaagtcctcctgactagactgacgtggcccttgctgtctagcctgctgcctggtggggttcaggctgccccgacaggtgcttcactctttcttgggctagtggaagaaagtgacaaacctcacatttgtgtaccaggcgtggaaatcatctcttgctgagtcccaccctccctgggctgaattcccgagtctctagacatttcttggcagagtctggcccagcctcaaccctaacccaggtcctgacccatttctatccctgaccctgacctctgctacgcttggggcagagagggcaggcaaggtcatctggaaggtgtggcgtcccgagcagaaagagagatgagcacattctctgttggacatgaagagtagtcggggggattggggaatggagttgttaggagtcatgtgtgacctacggacctgcctgtcttctaggcgatctgccatgattctgtgtccagtgtgggctggagctgtgatttgtttaacccttcagatccctaccctgaagatagagagtgaacctccttcctgaggggtattcctgggggtgaggcacgctctgagcgtgctcaagggttctgtctgctgatgtctgctttctgattaaagtgtctccttacaaaaaaaaaa

SEQ ID NO: 70 - protein, 147 aa, Rattus norvegicus; identical to SEQ ID NO: 68